(19) United States
(12) Patent Application Publication
(10) Pub. No.: US 2009/0306062 A1
(43) Pub. Date: Dec. 10, 2009

(54) 2,5-Disubstituted Piperidines

(76) Inventors: Peter Herold, Allschwil (CH); Robert Mah, Allschwil (CH); Stefan Stutz, Allschwil (CH); Vincenzo Tschinke, Allschwil (CH); Isabelle Lyothier, Allschwil (CH); Stjepan Jelakovic, Allschwil (CH); Michael Quirmbach, Basel (CH); Christiane Marti, Allschwil (CH)

Correspondence Address: WENDEROTH, LIND & PONACK, L.L.P., 1030 15th Street, N.W.,, Suite 400 East, Washington, DC, 20005-1503, US

(21) Appl. No.: 12/308,117

(22) PCT Filed: Jun. 7, 2007
(86) PCT No.: PCT/EP2007/055627

§ 371 Date: Dec. 8, 2008

(30) Foreign Application Priority Data
Jun. 8, 2006 (CH) 00937/06

(51) Int. Cl. A61K 31/5386; C07D 413/14; A61K 31/538; C07D 498/10

(52) U.S. Cl. 514/229.8; 544/105; 514/230.5; 544/71

(57) ABSTRACT

The application relates to 2,5-disubstituted piperidines, their preparation and use as medicines, especially as renin inhibitors, of the general formula (I) in which R, R¹ and X are each as defined in detail in the description, and pharmaceutical preparations comprising these compounds.

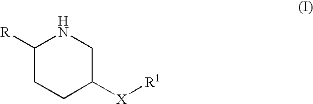

The present invention relates to the use of substituted piperidines as medicines, especially as renin inhibitors, to novel substituted piperidines, to processes for their preparation and to pharmaceutical preparations comprising the substituted piperidines.

Piperidine derivatives for use as medicines are known, for example, from WO 97/09311. With regard especially to renin inhibition, however, there is still a need for highly potent active ingredients. In this context, the improvement of the pharmacokinetic properties is at the forefront. These properties directed to better bioavailability are, for example, absorption, metabolic stability, solubility or lipophilicity.

The invention therefore provides for the use as medicines, especially as renin inhibitors, of 2,5-disubstituted piperidines of the general formula (I)

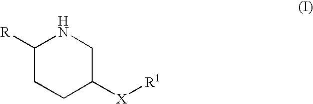

in which
R is C₂₋₈-alkenyl, C₂₋₈-alkynyl, C₁₋₈-alkyl, C₀₋₈-alkylcarbonyl-(N—C₀₋₈-alkyl)amino-C₁₋₈-alkyl, optionally N-mono- or N,N-di-C₁₋₈-alkylated or -arylated amino-C₁₋₈-alkyl, optionally O—C₁₋₈-alkylated carboxyl-C₀₋₈-alkyl, C₃₋₈-cycloalkyl-C₁₋₈-alkyl, optionally N-mono- or N,N-di-C₃₋₈-cycloalkyl-C₀₋₈-alkylated or optionally N-mono- or N,N-di-heterocyclyl-C₀₋₈-alkylated carbamoyl-C₀₋₈-alkyl, C₃₋₈-cycloalkyl-C₀₋₈-alkylcarbonyl-(N—C₀₋₈-alkyl)amino-C₁₋₈-alkyl, arylcarbonyl-(N—C₀₋₈-alkyl)amino-C₁₋₈-alkyl, optionally N-mono- or N,N-di-C₁₋₈-alkylated or -arylated sulphamoyl-C₀₋₈-alkyl, C₁₋₈-alkyl-sulphonyl-C₀₋₈-alkyl or heterocyclylcarbonyl-C₀₋₈-alkyl, each of which is either unsubstituted or substituted by 1-4 C₁₋₈-alkoxy, C₁₋₈-alkoxy-C₁₋₈-alkoxy, C₁₋₈-alkoxycarbonyl-(N—C₁₋₈-alkyl)amino, C₁₋₈-alkyl, C₁₋₈-alkylcarbonyl, C₀₋₈-alkylcarbonyl-(N—C₀₋₈-alkyl)amino, C₁₋₈-alkylsulphanyl, C₁₋₈-alkylsulphinyl, C₁₋₈-alkylsulphonyl, aryl-C₀₋₈-alkoxy, aryl, arylamino, aryl-C₀₋₈-alkylsulphonyl, cyano, C₀₋₈-cycloalkoxy, halogen, heterocyclyl, heterocyclyl-C₀₋₈-alkoxy, heterocyclyl-C₀₋₈-alkylamino, heterocyclylcarbonyl, hydroxyl, phosphonyl, optionally N-mono- or N,N-di-C₁₋₈-alkylated amino, optionally N-mono- or N,N-di-C₁₋₈-alkylated carbamoyloxy, optionally N-mono- or N,N-di-C₁₋₈-alkylated sulphamoyl, optionally N-mono-, -di- or -tri-C₁₋₈-alkylated or heterocyclyl-substituted ureido, optionally N-mono- or N,N-di-C₁₋₈-alkylated, N-mono- or N,N-di-arylated or N-mono- or N,N-di-heterocyclyl-C₀₋₈-alkylated amino-C₀₋₈-alkylcarbonyl, oxo or trifluoromethyl;
R¹ is aryl or heterocyclyl, each of which is substituted by 1-4 acyl-C₁₋₈-alkoxy-C₁₋₈-alkoxy, acyl-C₁₋₈-alkoxy-C₁₋₈-alkyl, (N-acyl)-C₁₋₈-alkoxy-C₁₋₈-alkylamino, C₁₋₈-alkanoyl, C₁₋₈-alkoxy, C₁₋₈-alkoxy-C₁₋₈-alkanoyl, C₁₋₈-alkoxy-C₁₋₈-alkoxy, C₁₋₈-alkoxy-C₁₋₈-alkoxy-C₁₋₈-alkyl, C₁₋₈-alkoxy-C₁₋₈-alkyl, (N—C₁₋₈-alkoxy)-C₁₋₈-alkylaminocarbonyl-C₁₋₈-alkoxy, (N—C₁₋₈-alkoxy)-C₁₋₈-alkylaminocarbonyl-C₁₋₈-alkyl, C₁₋₈-alkoxy-C₁₋₈-alkylcarbamoyl, C₁₋₈-alkoxy-C₁₋₈-alkylcarbonyl, C₁₋₈-alkoxy-C₁₋₈-alkylcarbonylamino, 1-C₁₋₈-alkoxy-C₁₋₈-alkylheterocyclyl, C₁₋₈-alkoxyaminocarbonyl-C₁₋₈-alkoxy, C₁₋₈-alkoxyaminocarbonyl-C₁₋₈-alkyl, C₁₋₈-alkoxycarbonyl, C₁₋₈-alkoxycarbonyl-C₁₋₈alkoxy, C₁₋₈-alkoxycarbonyl-C₁₋₈-alkyl, C₁₋₈-alkoxycarbonylamino-C₁₋₈-alkoxy, C₁₋₈-alkoxycarbonylamino-C₁₋₈alkyl, C₁₋₈-alkyl, (N—C₁₋₈-alkyl)-C₁₋₈-alkoxy-C₁₋₈-alkylcarbamoyl, (N—C₁₋₈-alkyl)-C₁₋₈-alkoxy-C₁₋₈-alkylcarbonylamino, (N—C₁₋₈-alkyl)-C₁₋₈-alkoxycarbonylamino, (N—C₁₋₈-alkyl)-C₁₋₈-alkylcarbonylamino-C₁₋₈-alkoxy, (N—C₁₋₈-alkyl)-C₁₋₈-alkylcarbonylamino-C₁₋₈-alkyl, (N—C₁₋₈-alkyl)-C₁₋₈-alkylsulphonylamino-C₁₋₈-alkoxy, (N—C₁₋₈-alkyl)-C₁₋₈-alkylsulphonylamino-C₁₋₈-alkyl, C₁₋₈-alkylamidinyl, C₁₋₈-alkylamino-C₁₋₈-alkoxy, di-C₁₋₈-alkylamino-C₁₋₈-alkoxy, C₁₋₈-alkylamino-C₁₋₈-alkyl, di-C₁₋₈-alkylamino-C₁₋₈alkyl, C₁₋₈-alkylaminocarbonyl-C₁₋₈-alkoxy, di-C₁₋₈-alkylaminocarbonyl-C₁₋₈-alkoxy, C₁₋₈-alkylaminocarbonyl-C₁₋₈-alkoxy-C₁₋₈-alkyl, C₁₋₈-alkylaminocarbonyl-C₁₋₈-alkyl, di-C₁₋₈-alkylaminocarbonyl-C₁₋₈-alkyl, C₁₋₈-alkylaminocarbonylamino-C₁₋₈-alkoxy, C₁₋₈-alkylaminocarbonylamino-C₁₋₈-alkyl, C₁₋₈-alkylcarbonylamino, C₁₋₈-alkylcarbonylamino-C₁₋₈-alkoxy, C₁₋₈-alkylcarbonylamino-C₁₋₈-alkyl, C₁₋₈-alkylcarbonyloxy-C₁₋₈-alkoxy, C₁₋₈-alkylcarbonyloxy-C₁₋₈-alkyl, C₁₋₈-alkylsulphonyl, C₁₋₈-alkylsulphonyl-C₁₋₈-alkoxy, C₁₋₈-alkylsulphonyl-C₁₋₈-alkyl, C₁₋₈-alkylsulphonylamino-C₁₋₈-alkoxy, C₁₋₈-alkylsulphonylamino-C₁₋₈-alkyl, optionally N-mono- or N,N-di-C₁₋₈-alkylated amino, aryl-C₀₋₈-alkoxy, aryl-C₀₋₈-alkyl, optionally N-mono- or N,N-di-C₁₋₈ alkylated carbamoyl-C₀₋₈-alkoxy, optionally N-mono- or N,N-di-C₁₋₈-alkylated carbamoyl-C₀₋₈-alkyl, carboxy-C₁₋₈-alkoxy, carboxy-C₁₋₈-alkoxy-C₁₋₈-alkyl, carboxy-C₁₋₈-alkyl, cyano, cyano-C₁₋₈-alkoxy, cyano-C₁₋₈-alkyl, C₃₋₈-cycloalkyl-C₁₋₈-alkoxy, C₃₋₈-cycloalkyl-C₁₋₈-alkyl, C₃₋₈-cycloalkylcarbonylamino-C₁₋₈-alkoxy, C₃₋₈-cycloalkylcarbonylamino-C₁₋₈-alkyl, O,N-dimethylhydroxylamino-C₁₋₈-alkyl, halogen, halogen-C₁₋₈-alkoxy, halogen-C₁₋₈-alkyl, halogen-aryl, heterocyclyl-C₀₋₈-alkoxy, heterocyclyl-C₀₋₈-alkyl, heterocyclylcarbonyl, hydroxy-C₁₋₈-alkoxy-C₁₋₈-alkoxy, hydroxy-C₁₋₈-alkoxy-C₁₋₈-alkyl, hydroxy-C₁₋₈-alkyl, O-methyloximyl-C₁₋₈-alkyl, oxide or oxo;
where, when R¹ is heterocyclyl and contains at least one saturated carbon atom, this heterocyclyl radical may additionally be substituted at a saturated carbon atom by a C₂₋₈-alkylene chain whose two ends are fixed on this saturated carbon atom and thus form a spirocycle, where one CH₂ group of the alkylene chain may be replaced by oxygen;
X is -Alk-, —O-Alk-, -Alk-O—, —O-Alk-O—, —S-Alk-, -Alk-S—, -Alk-NR²—, —NR²-Alk-, —C(O)—NR²—, -Alk-C(O)—NR²—, —C(O)—NR²-Alk-, -Alk-C(O)—NR²-Alk-, —NR²—C(O)—, -Alk-NR²—C(O)—, —NR²—C(O)-Alk-, -Alk-NR²—C(O)-Alk-, —O-Alk-C(O)—NR²—, —O-Alk-NR²—C(O)—, —S(O)₂—NR²—, -Alk-S(O)₂—NR²—, —S(O)₂—NR²-Alk-, -Alk-S(O)₂—NR²-Alk-, —NR²—S(O)₂—, -Alk-NR²—S(O)₂—, —NR²—S(O)₂-Alk- or -Alk-NR²—S(O)₂-Alk-, where Alk is C₁₋₈-alkylene which may optionally be substituted by halogen; and where
R² is hydrogen, C₁₋₈-alkyl, C₁₋₈-alkoxy-C₁₋₈-alkyl, acyl or aryl-C₁₋₈-alkyl;
and their pharmaceutically usable salts, prodrugs or compounds, in which one or more atoms have been replaced by their stable, non-radioactive isotopes.

The linkage of the above (and hereinafter) mentioned substituent —X— within the compound of the formula (I) starts from the piperidine ring with the substituent —X-being arranged from left to right when written as indicated above. For example, the fragment “—X—R¹” of the compound of the formula (I) with X meaning “—NR²—S(O)₂—” is: “—NR²—S(O)₂—R¹”.

The meaning of “C₀-alkyl” in the above (and hereinafter) mentioned C₀₋₈-alkyl groups is a bond or, if located at a terminal position, a hydrogen atom.

The meaning of “C₀-alkoxy” in the above (and hereinafter) mentioned C₀₋₈-alkoxy groups is “—O—” or, if located at a terminal position, an —OH group.

The invention further provides novel 2,5-disubstituted piperidines of the general formula (I)

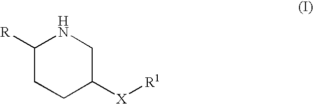

and salts, especially pharmaceutically usable salts, thereof, in which
R is C₂₋₈-alkenyl, C₂₋₈-alkynyl, C₁₋₈-alkyl, C₀₋₈-alkylcarbonyl-(N—C₀₋₈-alkyl)amino-C₁₋₈-alkyl, optionally N-mono- or N,N-di-C₁₋₈-alkylated or -arylated amino-C₁₋₈-alkyl, optionally O—C₁₋₈-alkylated carboxyl-C₀₋₈-alkyl, C₀₋₈-cycloalkyl-C₁₋₈-alkyl, optionally N-mono- or N,N-di-C₁₋₈-cycloalkyl-C₀₋₈-alkylated or optionally N-mono- or N,N-di-heterocyclyl-C₀₋₈-alkylated carbamoyl-C₀₋₈-alkyl, C₀₋₈-cycloalkyl-C₀₋₈-alkylcarbonyl-(N—C₀₋₈-alkyl)amino-C₁₋₈-alkyl, arylcarbonyl-(N—C₀₋₈-alkyl)amino-C₁₋₈-alkyl, optionally N-mono- or N,N-di-C₁₋₈-alkylated or -arylated sulphamoyl-C₀₋₈-alkyl, C₁₋₈-alkyl-sulphonyl-C₀₋₈-alkyl or heterocyclylcarbonyl-C₀₋₈-alkyl, each of which is either unsubstituted or substituted by 1-4 C₁₋₈-alkoxy, C₁₋₈-alkoxy-C₁₋₈-alkoxy, C₁₋₈-alkoxycarbonyl-(N—C₁₋₈-alkyl)amino, C₁₋₈-alkyl, C₁₋₈-alkylcarbonyl, C₀₋₈-alkylcarbonyl-(N—C₀₋₈-alkyl)amino, C₁₋₈-alkylsulphanyl, C₁₋₈-alkylsulphinyl, C₁₋₈-alkylsulphonyl, aryl-C₀₋₈-alkoxy, aryl, arylamino, aryl-C₀₋₈-alkylsulphonyl, cyano, CG-cycloalkoxy, halogen, heterocyclyl, heterocyclyl-C₀₋₈-alkoxy, heterocyclyl-C₀₋₈-alkylamino, heterocyclylcarbonyl, hydroxyl, phosphonyl, optionally N-mono- or N,N-di-C₁₋₈-alkylated amino, optionally N-mono- or N,N-di-C₁₋₈-alkylated carbamoyloxy, optionally N-mono- or N,N-di-C₁₋₈-alkylated sulphamoyl, optionally N-mono-, -di- or -tri-C₁₋₈-alkylated or heterocyclyl-substituted ureido, optionally N-mono- or N,N-di-C₁₋₈-alkylated, N-mono- or N,N-di-arylated or N-mono- or N,N-di-heterocyclyl-C₀₋₈-alkylated amino-C₀₋₈-alkylcarbonyl, oxo or trifluoromethyl;
R¹ is aryl or heterocyclyl, each of which is substituted by 1-4 acyl-C₁₋₈-alkoxy-C₁₋₈-alkoxy, acyl-C₁₋₈-alkoxy-C₁₋₈-alkyl, (N-acyl)-C₁₋₈-alkoxy-C₁₋₈-alkylamino, C₁₋₈-alkanoyl, C₁₋₈-alkoxy, C₁₋₈-alkoxy-C₁₋₈-alkanoyl, C₁₋₈-alkoxy-C₁₋₈-alkoxy, C₁₋₈-alkoxy-C₁₋₈-alkoxy-C₁₋₈-alkyl, C₁₋₈-alkoxy-C₁₋₈-alkyl, (N—C₁₋₈-alkoxy)-C₁₋₈-alkylaminocarbonyl-C₁₋₈-alkoxy, (N—C₁₋₈-alkoxy)-C₁₋₈-alkylaminocarbonyl-C₁₋₈-alkyl, C₁₋₈-alkoxy-C₁₋₈-alkylcarbamoyl, C₁₋₈-alkoxy-C₁₋₈-alkylcarbonyl, C₁₋₈-alkoxy-C₁₋₈-alkylcarbonylamino, 1-C₁₋₈-alkoxy-C₁₋₈-alkylheterocyclyl, C₁₋₈-alkoxyaminocarbonyl-C₁₋₈-alkoxy, C₁₋₈-alkoxyaminocarbonyl-C₁₋₈-alkyl, C₁₋₈-alkoxycarbonyl, C₁₋₈-alkoxycarbonyl-C₁₋₈-alkoxy, C₁₋₈-alkoxycarbonyl-C₁₋₈-alkyl, C₁₋₈-alkoxycarbonylamino-C₁₋₈-alkoxy, C₁₋₈-alkoxycarbonylamino-C₁₋₈-alkyl, C₁₋₈-alkyl, (N—C₁₋₈-alkyl)-C₁₋₈-alkoxy-C₁₋₈-alkylcarbamoyl, (N—C₁₋₈-alkyl)-C₁₋₈-alkoxy-C₁₋₈-alkylcarbonylamino, (N—C₁₋₈-alkyl)-C₁₋₈-alkoxycarbonylamino, (N—C₁₋₈-alkyl)-C₁₋₈-alkylcarbonylamino-C₁₋₈-alkoxy, (N—C₁₋₈-alkyl)-C₁₋₈-alkylcarbonylamino-C₁₋₈-alkyl, (N—C₁₋₈-alkyl)-C₁₋₈-alkylsulphonylamino-C₁₋₈-alkoxy, (N—C₁₋₈-alkyl)-C₁₋₈-alkylsulphonylamino-C₁₋₈-alkyl, C₁₋₈-alkylamidinyl, C₁₋₈-alkylamino-C₁₋₈-alkoxy, di-C₁₋₈-alkylamino-C₁₋₈-alkoxy, C₁₋₈-alkylamino-C₁₋₈-alkyl, di-C₁₋₈-alkylamino-C₁₋₈-alkyl, C₁₋₈-alkylaminocarbonyl-C₁₋₈-alkoxy, di-C₁₋₈-alkylaminocarbonyl-C₁₋₈-alkoxy, C₁₋₈-alkylaminocarbonyl-C₁₋₈-alkoxy-C₁₋₈-alkyl, C₁₋₈-alkylaminocarbonyl-C₁₋₈-alkyl, di-C₁₋₈-alkylaminocarbonyl-C₁₋₈-alkyl, C₁₋₈-alkylaminocarbonylamino-C₁₋₈-alkoxy, C₁₋₈-alkylaminocarbonylamino-C₁₋₈-alkyl, C₁₋₈-alkylcarbonylamino, C₁₋₈-alkylcarbonylamino-C₁₋₈-alkoxy, C₁₋₈-alkylcarbonylamino-C₁₋₈-alkyl, C₁₋₈-alkylcarbonyloxy-C₁₋₈-alkoxy, C₁₋₈-alkylcarbonyloxy-C₁₋₈-alkyl, C₁₋₈-alkylsulphonyl, C₁₋₈-alkylsulphonyl-C₁₋₈-alkoxy, C₁₋₈-alkylsulphonyl-C₁₋₈-alkyl, C₁₋₈-alkylsulphonylamino-C₁₋₈-alkoxy, C₁₋₈-alkylsulphonylamino-C₁₋₈-alkyl, optionally N-mono- or N,N-di-C₁₋₈-alkylated amino, aryl-C₀₋₈-alkoxy, aryl-C₀₋₈-alkyl, optionally N-mono- or N,N-di-C₁₋₈-alkylated carbamoyl-C₀₋₈-alkoxy, optionally N-mono- or N,N-di-C₁₋₈-alkylated carbamoyl-C₁₋₈-alkyl, carboxy-C₁₋₈-alkoxy, carboxy-C₁₋₈-alkoxy-C₁₋₈-alkyl, carboxy-C₁₋₈-alkyl, cyano, cyano-C₁₋₈-alkoxy, cyano-C₁₋₈-alkyl, C₃₋₈-cycloalkyl-C₁₋₈-alkoxy, C₃₋₈-cycloalkyl-C₁₋₈-alkyl, C₃₋₈-cycloalkylcarbonylamino-C₁₋₈-alkoxy, C₁₋₈-cycloalkylcarbonylamino-C₁₋₈-alkyl, O,N-dimethylhydroxylamino-C₁₋₈-alkyl, halogen, halogen-C₁₋₈-alkoxy, halogen-C₁₋₈-alkyl, halogen-aryl, heterocyclyl-C₀₋₈-alkoxy, heterocyclyl-C₀₋₈-alkyl, heterocyclylcarbonyl, hydroxy-C₁₋₈-alkoxy-C₁₋₈-alkoxy, hydroxy-C₁₋₈-alkoxy-C₁₋₈-alkyl, hydroxy-C₁₋₈-alkyl, O-methyloximyl-C₁₋₈-alkyl, oxide or oxo;
where, when R¹ is heterocyclyl and contains at least one saturated carbon atom, this heterocyclyl radical may additionally be substituted at a saturated carbon atom by a C₂₋₈-alkylene chain whose two ends are fixed on this saturated carbon atom and thus form a spirocycle, where one CH₂ group of the alkylene chain may be replaced by oxygen;
X is -Alk-, —O-Alk-, -Alk-O—, —O-Alk-O—, —S-Alk-, -Alk-S—, -Alk-NR²—, —NR²-Alk-, —C(O)—NR²-Alk-C(O)—NR²—, —C(O)—NR²-Alk-, -Alk-C(O)—NR²-Alk-, —NR²—C(O)—, -Alk-NR²—C(O)—, —NR²—C(O)-Alk-, -Alk-NR²—C(O)-Alk-, —O-Alk-C(O)—NR²—, —O-Alk-NR²—C(O)—, —S(O)₂—NR²—, -Alk-S(O)₂—NR²—, —S(O)₂—NR²-Alk-, -Alk-S(O)₂—NR²-Alk-, —NR²—S(O)₂—, -Alk-NR²—S(O)₂—, —NR²—S(O)₂-Alk- or -Alk-NR²—S(O)₂-Alk-, where Alk is C₁₋₈-alkylene which may optionally be substituted by halogen; where
R² is hydrogen, C₁₋₈-alkyl, C₁₋₈-alkoxy-C₁₋₈-alkyl, acyl or aryl-C₁₋₈-alkyl;
and where,
when X is —C(O)—NR²—, -Alk-C(O)—NR²—, —C(O)—NR²-Alk-, -Alk-C(O)—NR²-Alk-, —NR²—C(O)—, -Alk-NR²—C(O)—NR²—C(O)-Alk-, -Alk-NR²—C(O)-Alk-, —O-Alk-C(O)—NR²—, —O-Alk-NR²—C(O)—, R is not a —CO-bonded substituent;
when X is -Alk-NR²—C(O)—, —NR²—C(O)— and R is an O—C₁₋₈-alkylated carboxyl-C₀₋₈-alkyl, C₁₋₈-alkyl, C₀₋₈-alkylcarbonyl-(N—C₀₋₈-alkyl)amino-C₁₋₈-alkyl, optionally N-mono- or N,N-di-C₁₋₈-alkylated or -arylated amino-C₁₋₈-alkyl, or C₃₋₈-cycloalkyl-C₀₋₈-alkylated or heterocyclyl-C₀₋₈-alkylated carbamoyl-C₀₋₈-alkyl, each of which is optionally substituted, —C(O)—R¹ is not a substituent which stems from a carbonylation reagent;
when X is -Alk-NR²—, —NR²-Alk- or —NR²—CO-Alk- and R¹ is an optionally substituted aryl, R is not a diphenylmethyl substituent or a phenyl(heteroaryl)methyl substituent, each of which is optionally substituted;
when X is -Alk-NR²—S(O)₂—, R is not an optionally N-mono- or N,N-di-C₁₋₈-alkylated or -arylated amino-C₁₋₈-alkyl substituent;
when X is —NR²—C(O)— and R¹ is an optionally substituted aryl, R is not a C₂₋₈-alkenyl substituent, a C₁₋₈-alkyl substituent, a C₂₋₈-alkynyl substituent or a C₃₋₈-cycloalkyl-C₁₋₈-alkyl substituent, each of which is optionally substituted;
when R is C₁₋₈-alkyl- or arylcarbonyl-(N—C₀₋₈-alkyl)amino-C₁₋₈-alkyl, X is not -Alk-;
when X is -Alk-C(O)—NR²— and R¹ is a phenyl substituted by 0-2 methoxy, R is not a C₀₋₈-alkyl- or arylcarbonyl-(N—C₀₋₈-alkyl)amino-C₁₋₈-alkyl substituent or optionally O—C₁₋₈-alkylated carboxyl-C₀₋₈-alkyl substituent;
when X is -Alk- and R¹ is an amino-substituted monocyclic nitrogen heteroaryl which may optionally be substituted, R is not a C₁₋₈-alkyl;
when X is -Alk-, —C(O)—NR²— or —NR²—C(O)— and R¹ is an optionally substituted pyridine, R is not a C₁₋₈-alkyl substituent, an optionally O—C₁₋₈-alkylated carboxyl-C₀₋₈-alkyl substituent or an optionally N-mono- or N,N-di-C₃₋₈-cycloalkyl-C₀₋₈-alkylated carbamoyl-C₀₋₈-alkyl substituent;
when R is an optionally substituted O—C₁₋₈-alkylated carboxyl-C₀₋₈-alkyl, R¹ is not a 4-aminopyrido[3,2-d]pyrimidin-6-yl substituent, a 4-aminopyrido[3,4-d]pyrimidin-6-yl substituent, a 4-aminopyrido[4,3-d]pyrimidin-7-yl substituent, a 4-aminopyrido[2,3-d]pyrimidin-7-yl substituent, a 4-aminopyrimido[4,5-d]pyrimidin-7-yl substituent, or a 4-aminopyrimido[5,4-d]pyrimidin-6-yl substituent, each of which is optionally substituted;
when X is —C(O)—NR²— and R¹ is an optionally substituted 2-benzothiazole, R is not a C₁₋₈-alkyl substituent, a C₁₋₈-haloalkyl substituent or a carboxyl-C₀₋₈-alkyl substituent;
when X is —NR²-Alk- and R¹ is phenyl, R is not an optionally substituted C₀₋₈-alkylated carbamoyl-C₀₋₈-alkyl substituent or an optionally N-mono- or N,N-di-C₁₋₈-alkylated or -arylated amino-C₁₋₈-alkyl substituent;
when X is -Alk-O— or -Alk-NR²— and R¹ is pyrazolyl, R is not C₁₋₈-alkyl;
when X is —NR²-Alk- and R¹ is optionally substituted phenyl, R is not a hydroxy- or C₁₋₈-alkoxy-substituted C₁₋₈-alkyl substituent;
when X is —NR²—C(O)— or —NR²—C(O)-Alk- and R is indol-3-yl-C₁₋₈-alkyl, R¹ is not an aryl substituent;
when X is -Alk- and R¹ is an optionally substituted phenyl, R is not an optionally hydroxy-, cyano-, C₁₋₈-alkylcarbonyl- or aryl-substituted C₂₋₈-alkenyl, C₁₋₈-alkyl, C₂₋₈-alkynyl, C₃₋₈-cycloalkyl-C₁₋₈-alkyl or optionally O—C₁₋₈-alkylated carboxyl-C₀₋₈-alkyl;
when X is -Alk-O—, —O-Alk-, Alk-S—, —S-Alk-, Alk-NR²—, —NR²-Alk-, —CO—NR²— or —NR²—CO— and R is C₁₋₈-alkyl, R¹ is not a 9-membered [4.3.0]-bicycle;
when X is —CO—NR²— and R¹ is an optionally substituted aryl, R is not an optionally substituted C₁₋₈-alkyl substituent, a carbamoyl substituent, a heteroarylcarbonyl substituent or an optionally N-mono- or N,N-di-C₁₋₈-alkylated sulphamoyl substituent;
when X is —NR²—CO— and R is a C₁₋₈-alkyl, R¹ is not an optionally substituted 1-naphthyl;
when X is —S-Alk- or —O-Alk- and R¹ is an optionally substituted aryl, R is not a C₁₋₈-alkyl substituent, an optionally N-mono- or N,N-di-C₁₋₈-alkylated amino-C₁₋₈-alkyl substituent or an optionally O—C₁₋₈-alkylated carboxyl-C₀₋₈-alkyl substituent, each of which is optionally substituted by hydroxyl, alkoxy or oxo;
when X is —NR²—(CH)₂, R is not a C₁₋₈-alkyl substituent, an optionally N-mono- or N,N-di-C₁₋₈alkylated amino-C₁₋₈-alkyl substituent or an optionally O—C₁₋₈-alkylated carboxyl-C₀₋₈-alkyl substituent, each of which is optionally substituted by hydroxyl, alkoxy or oxo;
when X is -Alk-NR²—CO— and R¹ is an optionally substituted 3-quinolin-4-olyl, R is not C₁₋₈-alkyl;
when R is an optionally O—C₁₋₈-alkylated carboxyl-C₀₋₈-alkyl or an optionally N-mono- or N,N-di-C₁₋₈-alkylated, C₃₋₈-cycloalkyl-C₀₋₈-alkylated or heterocyclyl-C₀₋₈-alkylated carbamoyl-C₀₋₈-alkyl, X is not -Alk-;
when X is —O-Alk-C(O)—NR²—, -Alk-C(O)—NR²—, —C(O)—NR²—, -Alk-S(O)₂—NR²— or —S(O)₂—NR²—, R is not a C₁₋₈-alkyl substituent, an optionally N-mono- or N,N-di-C₁₋₈-alkylated or -arylated amino-C₁₋₈-alkyl substituent, or a C₃₋₈-cycloalkyl-C₁₋₈-alkyl substituent, each of which are optionally substituted; and
when X is —O-Alk- or -Alk-O— and R¹ is an optionally substituted phenyl, R is not a hydroxy-substituted C₀₋₈-alkylcarbonyl-(N—C₀₋₈-alkyl)amino-C₁₋₈-alkyl substituent.

Examples of C₁₋₈-alkyl and -alkoxy radicals are, respectively, methyl, ethyl, n-propyl, isopropyl, n-butyl, isobutyl, sec-butyl, tert-butyl, pentyl, hexyl, and methoxy, ethoxy, propoxy, isopropoxy, butoxy, isobutoxy, sec-butoxy and tert-butoxy. C₁₋₈-Alkylenedioxy radicals are preferably methylenedioxy, ethylenedioxy and propylenedioxy. Examples of C₁₋₈-alkanoyl radicals are acetyl, propionyl and butyryl. Cycloalkyl is a saturated cyclic hydrocarbon radical having 3-12 carbon atoms, i.e. cyclopropyl, cyclobutyl, cyclopentyl and cyclohexyl, cycloheptyl, bicyclo[2.2.1]heptyl, cyclooctyl, bicyclo[2.2.2]octyl and adamantyl, which may be mono- or polysubstituted. Examples of substituents on such cycloalkyl radicals are C₁₋₈-alkoxy, C₁₋₈-alkyl, C₁₋₈-alkylcarbonyloxy, carbamoyl, carboxyl, cyano, halogen, hydroxyl, oxo, trifluoromethoxy or trifluoromethyl. C₁₋₈-Alkylene radicals may be linear or branched and are, for example, methylene, ethylene, propylene, 2-methylpropylene, 2-methylbutylene, 2-methylpropyl-2-ene, butyl-2-ene, butyl-3-ene, propyl-2-ene, tetra-, penta- and hexamethylene; C₂₋₈-alkenylene radicals are, for example, vinylene and propenylene; an example of a C₂₋₈-alkynylene radical is ethynylene; acyl radicals are alkanoyl radicals, preferably C₁₋₈-alkanoyl radicals, or aroyl radicals such as benzoyl.

Aryl denotes mono- or polycyclic aromatic radicals which may be mono- or polysubstituted, for example phenyl, substituted phenyl, naphthyl or substituted naphthyl. Examples of substituents on such aryl radicals are C₁₋₈-alkyl, trifluoromethyl, trifluoromethoxy, nitro, amino, C₂₋₈-alkenyl, C₁₋₈-alkylsulphinyl, C₁₋₈-alkoxy, C₁₋₈-alkylcarbonyloxy, hydroxyl, halogen, cyano, carbamoyl, carboxyl and C₁₋₈-alkylenedioxy, and also optionally halogen-, C₁₋₈-alkyl-, C₁₋₈-alkoxy- or dihydroxy-C₁₋₈-alkylaminocarbonyl-substituted phenyl, phenoxy, phenylthio, phenyl-C₁₋₈-alkyl or phenyl-C₁₋₈-alkoxy. Further examples of substituents on aryl or heterocyclyl radicals are oxide, oxo, C₁₋₈-alkoxycarbonylphenyl, hydroxy-C₁₋₈-alkylphenyl, benzyloxy, pyridylcarbonylamino-C₁₋₈-alkyl, C₂₋₈-alkenyloxy, C₁₋₈-alkoxy-C₁₋₈-alkoxy, C₁₋₈-alkoxy-C₁₋₈-alkoxy-C₁₋₈-alkyl, hydroxy-C₁₋₈-alkoxy, di-C₁₋₈-alkylamino, 2,3-dihydroxypropoxy, 2,3-dihydroxypropoxy-C₁₋₈-alkoxy, 2,3-dimethoxypropoxy, methoxybenzyloxy, hydroxybenzyloxy, phenethyloxy, methylenedioxybenzyloxy, dioxolanyl-C₁₋₈-alkoxy, cyclopropyl-C₁₋₈-alkyl, cyclopropyl-C₁₋₈-alkoxy, hydroxy-C₁₋₈-alkoxy, carbamoyloxy-C₁₋₈-alkoxy, pyridylcarbamoyloxy-C₁₋₈-alkoxy, benzoyloxy-C₁₋₈-alkoxy, picolyloxy, C₁₋₈-alkoxycarbonyl, C₁₋₈-alkylcarbonylamino, C₁₋₈-alkylcarbonylamino-C₁₋₈-alkyl, C₀₋₈-alkylcarbonylamino-C₁₋₈-alkoxy, (N—C₁₋₈-alkyl)-C₁₋₈-alkylcarbonylamino-C₁₋₈-alkyl, (N—C₁₋₈-alkyl)-C₁₋₈-alkylcarbonylamino-C₁₋₈-alkoxy, C₃₋₈-cycloalkylcarbonylamino-C₁₋₈-alkyl, C₃₋₈-cycloalkylcarbonylamino-C₁₋₈-alkoxy, C₁₋₈-alkoxy-C₁₋₈-alkyl, hydroxy-C₁₋₈-alkyl, hydroxy-C₁₋₈-alkoxy-C₁₋₈-alkyl, hydroxy-C₁₋₈-alkoxy-C₁₋₈-alkoxy, C₁₋₈-alkoxycarbonylamino-C₁₋₈-alkyl, C₁₋₈-alkoxycarbonylamino-C₁₋₈-alkoxy, C₁₋₈-alkylaminocarbonylamino-C₁₋₈-alkyl, C₁₋₈-alkylaminocarbonylamino-C₁₋₈-alkoxy, C₁₋₈-alkylaminocarbonyl-C₁₋₈-alkyl, C₁₋₈-alkylaminocarbonyl-C₁₋₈-alkoxy, C₁₋₈-alkylaminocarbonyl-C₁₋₈-alkoxy-C₁₋₈-alkyl, di-C₁₋₈-alkylaminocarbonyl-C₁₋₈-alkyl, di-C₁₋₈-alkylaminocarbonyl-C₁₋₈-alkoxy, C₁₋₈-alkylcarbonyloxy-C₁₋₈-alkyl, C₁₋₈-alkylcarbonyloxy-C₁₋₈-alkoxy, cyano-C₁₋₈-alkyl, cyano-C₁₋₈-alkoxy, 2-oxooxazolidinyl-C₁₋₈-alkyl, 2-oxo-oxazolidinyl-C₁₋₈-alkoxy, C₁₋₈-alkoxycarbonyl-C₁₋₈-alkyl, C₁₋₈-alkoxycarbonyl-C₁₋₈-alkoxy, C₁₋₈-alkylsulphonylamino-C₁₋₈-alkyl, C₁₋₈-alkylsulphonylamino-C₁₋₈-alkoxy, (N—C₁₋₈-alkyl)-C₁₋₈-alkylsulphonylamino-C₁₋₈-alkyl, (N—C₁₋₈-alkyl)-C₁₋₈-alkylsulphonylamino-C₁₋₈-alkoxy, amino-C₁₋₈-alkyl, amino-C₁₋₈-alkoxy, C₁₋₈-alkylamino-C₁₋₈-alkyl, C₁₋₈-alkylamino-C₁₋₈-alkoxy, di-C₁₋₈-alkylamino-C₁₋₈-alkyl, di-C₁₋₈-alkylamino-C₁₋₈-alkoxy, C₁₋₈-alkylsulphonyl-C₁₋₈-alkyl, C₁₋₈-alkylsulphonyl-C₁₋₈-alkoxy, carboxy-C₁₋₈-alkyl, carboxy-C₁₋₈-alkoxy, carboxy-C₁₋₈-alkoxy-C₁₋₈-alkyl, C₁₋₈-alkoxy-C₁₋₈-alkylcarbonyl, acyl-C₁₋₈-alkoxy-C₁₋₈-alkyl, (N—C₁₋₈-alkyl)-C₁₋₈-alkoxycarbonylamino, (N-hydroxy)-C₁₋₈-alkylaminocarbonyl-C₁₋₈-alkyl, (N-hydroxy)-C₁₋₈-alkylaminocarbonyl-C₁₋₈-alkoxy, (N-hydroxy)aminocarbonyl-C₁₋₈-alkyl, (N-hydroxy)aminocarbonyl-C₁₋₈-alkoxy, C₁₋₈-alkoxyaminocarbonyl-C₁₋₈-alkyl, C₁₋₈-alkoxyaminocarbonyl-C₁₋₈-alkoxy, (N—C₁₋₈-alkoxy)-C₁₋₈-alkylaminocarbonyl-C₁₋₈-alkyl, (N—C₁₋₈-alkoxy)-C₁₋₈-alkylaminocarbonyl-C₁₋₈-alkoxy, (N-acyl)-C₁₋₈-alkoxy-CO₁-alkylamino, C₁₋₈-alkoxy-C₁₋₈-alkylcarbamoyl, (N—C₁₋₈-alkyl)-C₁₋₈-alkoxy-C₁₋₈-alkylcarbamoyl C₁₋₈-alkoxy-C₁₋₈-alkylcarbonyl, C₁₋₈-alkoxy-C₁₋₈-alkylcarbonylamino, (N—C₁₋₈-alkyl)-C₁₋₈-alkoxy-C₁₋₈-alkylcarbonylamino, 1-C₁₋₈-alkoxy-C₁₋₈-alkylimidazol-2-yl, 1-C₁₋₈-alkoxy-C₁₋₈-alkyltetrazol-5-yl, 5-C₁₋₈-alkoxy-C₁₋₈-alkyltetrazol-1-yl, 2-C₁₋₈-alkoxy-C₁₋₈-alkyl-4-oxoimidazol-1-yl, carbamoyl-C₁₋₈-alkyl, carbamoyl-C₁₋₈-alkoxy, C₁₋₈-alkylcarbamoyl, di-C₁₋₈-alkylcarbamoyl, C₁₋₈-alkylsulphonyl, C₁₋₈-alkylamidinyl, acetamidinyl-C₁₋₈-alkyl, O-methyloximyl-C₁₋₈-alkyl, O, N-dimethylhydroxylamino-C₁₋₈-alkyl, C₃₋₁₂-cycloalkyl-C₁₋₈-alkanoyl, aryl-C₁₋₈-alkanoyl, heterocyclyl-C₁₋₈-alkanoyl; and optionally halogen-, C₁₋₈-alkyl-, C₁₋₈-alkoxy- or dihydroxy-C₁₋₈-alkylaminocarbonyl-substituted pyridyl, pyridyloxy, pyridylthio, pyridylamino, pyridyl-C₁₋₈-alkyl, pyridyl-C₁₋₈-alkoxy, pyrimidinyl, pyrimidinyloxy, pyrimidinylthio, pyrimidinylamino, pyrimidinyl-C₁₋₈-alkyl, pyrimidinyl-C₁₋₈-alkoxy, thienyl, thienyl-C₁₋₈-alkyl, thienyl-C₁₋₈-alkoxy, furyl, furyl-C₁₋₈-alkyl, furyl-C₁₋₈-alkoxy.

The expression heterocyclyl denotes mono- or bicyclic, saturated and unsaturated heterocyclic radicals having 1 to 4 nitrogen and/or 1 or 2 sulphur or oxygen atoms, which may each be mono- or polysubstituted, especially by (in the case of unsaturated heterocyclyl radicals) alkyl, hydroxyl, alkoxy, nitro or halogen, or by substituents as defined above for aryl radicals, or (in the case of saturated heterocyclyl radicals) may be substituted by alkyl or alkoxy. Examples of heterocyclyl radicals are benzimidazolyl, benzo[1,3]dioxolyl, benzofuranyl, benzoxazolyl, benzothiazolyl, benzo[b]thienyl, quinazolinyl, quinolyl, quinoxalinyl, 2H-chromenyl, carbazolyl, dihydro-2H-benzo[1,4]oxazinyl, dihydro-3H-benzo[1,4]oxazinyl, dihydro-2H-benzo[1,4]thiazinyl, 2,3-dihydroindolyl, dihydro-1H-pyrido[2,3-b][1,4]oxazinyl, furyl, imidazolyl, imidazo[1,2-a]pyridyl, imidazo[1,5-a]pyridyl, indazolyl, indolyl, isobenzofuranyl, isoquinolyl, [1,5]naphthyridyl, oxazolyl, phthalazinyl, pyranyl, pyrazinyl, pyridyl, pyrimidinyl, 1H-pyrrolizinyl, 1H-pyrrolo[2,3-b]pyridyl, 1H-pyrrolo[2,3-c]pyridyl, 1H-pyrrolo[3,2-b]pyridyl, pyrrolyl, tetrahydroquinolyl, tetrahydroquinoxalinyl, tetrahydroimidazo[1,2-a]pyridyl, tetrahydroimidazo[1,5-a]pyridyl, tetrahydroisoquinolyl, thiazolyl, thienyl, [1,2,3]triazolo[1,5-a]pyridyl, [1,2,4]triazolo[4,3-a]pyridyl or triazolyl. Examples of substituted heterocyclyl radicals are 2,2-dimethyl-3-oxo-4H-benzo[1,4]oxazinyl, 2,2-dimethyl-3,4-dihydro-2H-benzo[1,4]oxazinyl, 2-aryl-2-methyl-3,4-dihydro-2H-benzo[1,4]oxazinyl, 2,2-dimethyl-2H-chromen-6-yl, 2-aryl-2-methyl-2H-chromen-6-yl, 2-oxobenzoimidazolyl, 2-oxodihydrobenzo[d][1,3]oxazinyl, 4-oxodihydroimidazolyl, 5-oxo-4H-[1,2,4]triazinyl, 3-oxo-4H-benzo[1,4]thiazinyl, 1,1,3-trioxodihydro-2H-1λ⁶-benzo[1,4]thiazinyl, 1-oxopyridyl, 2-oxotetrahydrobenzo[e][1,4]diazepinyl, 2-oxodihydrobenzo[e][1,4]diazepinyl, 1-oxo-3H-isobenzofuranyl, 4-oxo-3H-thieno[2,3-d]pyrimidinyl, 3-oxo-4H-benzo[1,4]oxazinyl, 1,1-dioxodihydro-2H-benzo[1,4]thiazinyl, 2-oxo-1H-pyrido[2,3-b][1,4]oxazinyl, 2-oxobenzooxazolyl, 2-oxo-1,3-dihydroindolyl, 2-oxodihydro-1H-quinazolinyl, nitrobenzothiazolyl, phenyltetrazolyl, phenyloxadiazolyl, phenylpiperidinyl, phenylpiperazinyl, phenylpyrrolidinyl, thienyloxadiazolyl, furanyloxadiazolyl, benzyloxadiazolyl or phenyloxazolyl. Examples of saturated heterocyclyl radicals are azetidinyl, dioxolanyl, dioxanyl, dithiolanyl, dithianyl, pyrrolidinyl, piperidinyl, piperazinyl, 4-methylpiperazinyl, morpholinyl, thiomorpholinyl, 2-hydroxymethylpyrrolidinyl, 3-hydroxypyrrolidinyl, 3,4-dihydroxypyrrolidinyl, 4-hydroxypiperidinyl, 4-oxopiperidinyl, 3,5-dimethylmorpholinyl, 4,4-dioxothiomorpholinyl, 4-oxothiomorpholinyl, 2,6-dimethylmorpholinyl, tetrahydropyranyl, 2-oxoimidazolidinyl, 2-oxooxazolidinyl, 2-oxopiperidinyl, 2-oxopyrrolidinyl, 2-oxo[1,3]oxazinyl, 2-oxoazepanyl, 2-oxotetrahydropyrimidinyl and the like. Examples of bicyclic heterocyclyl radicals are 2-oxabicyclo[4.1.0]heptanyl, 3-oxabicyclo[4.1.0]heptanyl, 2-oxabicyclo[3.1.0]hexanyl or 3-oxabicyclo[3.1.0]hexanyl.

In the case of R, R¹ and R², the aryl, aroyl and heterocyclyl radicals may additionally be substituted by heterocyclylalkyl, heterocyclylalkoxy, heterocyclylalkoxyalkyl or heterocyclyl, for example piperidinoalkyl, piperidinoalkoxy, piperidinoalkoxyalkyl, morpholinoalkyl, morpholinoalkoxy, morpholinoalkoxyalkyl, piperazinoalkyl, piperazinoalkoxy, piperazinoalkoxyalkyl, [1,2,4]-triazol-1-ylalkyl, [1,2,4]-triazol-1-ylalkoxy, [1,2,4]-triazol-4-ylalkyl, [1,2,4]-triazol-4-ylalkoxy, [1,2,4]-oxadiazol-5-ylalkyl, [1,2,4]-oxadiazol-5-ylalkoxy, 3-methyl-[1,2,4]-oxadiazol-5-ylalkyl, 3-methyl-[1,2,4]-oxadiazol-5-ylalkoxy, 5-methyl-[1,2,4]-oxadiazol-3-ylalkyl, 5-methyl-[1,2,4]-oxadiazol-3-ylalkoxy, tetrazol-1-ylalkyl, tetrazol-1-ylalkoxy, tetrazol-2-ylalkyl, tetrazol-2-ylalkoxy, tetrazol-5-ylalkyl, tetrazol-5-ylalkoxy, 5-methyltetrazol-1-ylalkyl, 5-methyltetrazol-1-ylalkoxy, thiazol-4-ylalkyl, thiazol-4-ylalkoxy, oxazol-4-ylalkyl, oxazol-4-ylalkoxy, 2-oxopyrrolidinylalkyl, 2-oxopyrrolidinylalkoxy, imidazolylalkyl, imidazolylalkoxy, 2-methylimidazolylalkyl, 2-methylimidazolylalkoxy or N-methylpiperazinoalkyl, N-methylpiperazinoalkoxy, N-methylpiperazinoalkoxyalkyl, and also alkylaminoalkyl, alkylaminoalkoxy, alkylaminoalkoxyalkyl, mono- and polyhydroxyalkyl, -alkoxy, -alkoxyalkyl and -alkoxyalkoxy, carbamoylalkyloxy, C₁₋₈-alkoxy, amino-C₁₋₈-alkoxy, hydroxy-C₁₋₈-alkoxy, dioxolanyl, dioxanyl, dithiolanyl, dithianyl, pyrrolidinyl, piperidinyl, piperazinyl, pyrrolyl, 4-methylpiperazinyl, morpholinyl, thiomorpholinyl, 2-hydroxymethylpyrrolidinyl, 3-hydroxypyrrolidinyl, 3,4-dihydroxypyrrolidinyl, 3-acetamidomethylpyrrolidinyl, 3-C₁₋₈-alkoxy-C₁₋₈-alkylpyrrolidinyl, 4-hydroxypiperidinyl, 4-oxopiperidinyl, 3,5-dimethylmorpholinyl, 4,4-dioxothiomorpholinyl, 4-oxothiomorpholinyl, 2,6-dimethylmorpholinyl, 2-oxoimidazolidinyl, 2-oxooxazolidinyl, 2-oxopyrrolidinyl, 2-oxo-[1,3]oxazinyl, 2-oxotetrahydropyrimidinyl and the like, or by the —O—CH₂CH(OH)CH₂NRx radical, where NRx is a mono- or di-C₁₋₈-alkylamino, piperidino, morpholino, piperazino or N-methylpiperazino radical.

The expression polyhydroxyalkyl denotes C₁₋₇-alkyl radicals which may be substituted by 2-8 hydroxyl groups, for example glyceryl, arabityl, sorbityl, etc.

The expression halogen or halo denotes, for example, fluorine, chlorine or bromine, or a radical mono- or polysubstituted by fluorine, chlorine or bromine.

The compounds of the formula (I) have at least two asymmetric carbon atoms and may therefore be present in the form of optically pure diastereomers, diastereomer mixtures, diastereomeric racemates, mixtures of diastereomeric racemates or as meso compounds. The invention encompasses all of these forms. Diastereomer mixtures, diastereomeric racemates or mixtures of diastereomeric racemates may be separated by customary methods, for example by column chromatography, thin-layer chromatography, HPLC and the like.

The configuration of an individual chiral centre in a compound of the formula (I) may be inverted selectively. For example, the configuration of an asymmetric carbon which bears nucleophilic substituents, for example amino or hydroxyl group, can be inverted by a bimolecular nucleophilic substitution with a reagent which reintroduces the original substituent, optionally with preceding conversion of the bonded nucleophilic radical to a suitable nucleofugic leaving group. Alternatively, the configuration of an asymmetric carbon which bears a hydroxyl group can be inverted by oxidation and reduction analogously to the method described in the European patent application EP-A-0 236 734. Equally advantageous is the functionalization of the hydroxyl group to a more reactive species, followed by a substitution of this group by a hydroxyl group with inversion of configuration.

Salts are principally pharmaceutically usable salts or non-toxic salts of the compounds of the formula (I). The expression “pharmaceutically usable salts” encompasses salts with inorganic or organic acids, such as hydrochloric acid, hydrobromic acid, nitric acid, sulphuric acid, phosphoric acid, citric acid, formic acid, maleic acid, acetic acid, succinic acid, tartaric acid, methanesulphonic acid, p-toluenesulphonic acid and the like.

For the purposes of isolation and purification, pharmaceutically unsuitable salts may also find use.

Salts of compounds with salt-forming groups result principally from the addition of an acid or of a base. If a plurality of salt-forming groups are present, it is also possible for mixed salts or internal salts to be present.

Such salts form, for example, from compounds of the formula (I) which contain an acidic functional group, for example a carboxyl group, and are, for example, salts of this functional group with a suitable base, for example non-toxic metal salts of metals of groups Ia, Ib, IIa and IIb of the Periodic Table, for example alkali metal salts, especially lithium, sodium or potassium salts, alkaline earth metal salts, especially magnesium or calcium salts, but also zinc salts and ammonium salts; also included are salts which form with organic amines, such as optionally hydroxy-substituted mono-, di- or trialkylamines, especially with mono-, di- or tri(lower alkyl)amines or with quaternary ammonium bases, for example methyl-, ethyl-, diethyl- or triethylamine, mono-, bis- or tris(2-hydroxy(lower alkyl))amines, for example ethanol-, diethanol- or triethanolamine, tris(hydroxymethyl)methylamine or 2-hydroxy-tert-butylamine, N,N-di(lower alkyl)-N-hydroxy(lower alkyl))amines, for example N,N-dimethyl-N-(2-hydroxyethyl)amine or N-methyl-D-glucamine, or quaternary ammonium hydroxides, for example tetrabutylammonium hydroxide. The compounds of the formula (I) which contain a basic functional group, for example an amino group, may form salts with acids, for example with suitable inorganic acids, for example hydrohalic acid, for example hydrochloric acid or hydrobromic acid, sulphuric acid with exchange of one or both protons, phosphoric acid with exchange of one or more protons, for example ortho- or metaphosphoric acid, pyrophosphoric acid with exchange of one or more protons, or with organic carboxylic acids, sulphonic acids or phosphoric acids or N-substituted sulphamic acids, for example acetic acid, propionic acid, glycolic acid, succinic acid, maleic acid, hydroxymaleic acid, methylmaleic acid, fumaric acid, malic acid, tartaric acid, gluconic acid, glucaric acid, glucuronic acid, citric acid, benzoic acid, cinnamic acid, mandelic acid, salicylic acid, 4-aminosalicylic acid, 2-phenoxybenzoic acid, 2-acetoxybenzoic acid, pamoic acid, nicotinic acid, isonicotinic acid, and also amino acids, for example the above-mentioned alpha-amino acids, but also methanesulphonic acid, ethanesulphonic acid, 2-hydroxyethanesulphonic acid, ethane-1,2-disulphonic acid, benzenesulphonic acid, 4-methylbenzenesulphonic acid, naphthol-2-benzenesulphonic acid, 2- or 3-phosphoglycerate, glucose-6-phosphate, N-cyclohexylsulphamic acid (with formation of the cyclamates) or with other organic acidic compounds, for example ascorbic acid. Compounds of the formula (I) which have acidic and basic functional groups may also form internal salts.

Salts may be obtained from other salts by known methods. Salts which arise in a formal sense by addition of an acid may be obtained, for example, by treatment with a suitable metal salt, for example the sodium, barium or silver salt of another acid in a suitable solvent in which the inorganic salt which forms is insoluble and is thus removed from the reaction equilibrium, and salts which arise in a formal sense by addition of a base by conversion to the free acid and salt reformation.

The compounds of the formula (I), including their salts, may also be obtained in the form of the hydrates or include the solvent from which they have been recrystallized.

Preferred inventive compounds are those of the general formula (IA)

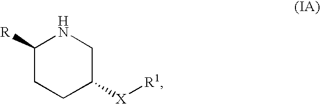

in which R, R¹ and X are each as defined above for the compounds of the formula (I).

A further preferred group of compounds of the formula (I), and particularly preferably of the formula (IA), and the salts thereof, preferably the pharmaceutically usable salts thereof, are compounds in which

R is C₂₋₈-alkenyl, C₂₋₈-alkynyl, C₁₋₈-alkyl, C₀₋₈-alkylcarbonyl-(N—C₀₋₈-alkyl)amino-C₁₋₈-alkyl, optionally N-mono- or N,N-di-C₁₋₈-alkylated or -arylated amino-C₁₋₈-alkyl, optionally O—C₁₋₈-alkylated carboxyl-C₀₋₈-alkyl, C₃₋₈-cycloalkyl-C₁₋₈-alkyl, optionally N-mono- or N,N-di-C₁₋₈-cycloalkyl-C₀₋₈-alkylated or optionally N-mono- or N,N-di-heterocyclyl-C₀₋₈-alkylated carbamoyl-C₀₋₈-alkyl, C₃₋₈-cycloalkyl-C₁₋₈-alkylcarbonyl-(N—C₀₋₈-alkyl)amino-C₁₋₈-alkyl, arylcarbonyl-(N—C₀₋₈-alkyl)amino-C₁₋₈-alkyl, optionally N-mono- or N,N-di-C₁₋₈-alkylated or -arylated sulphamoyl-C₀₋₈-alkyl, C₁₋₈-alkyl-sulphonyl-C₀₋₈-alkyl or heterocyclylcarbonyl-C₀₋₈-alkyl, each of which is either un-substituted or substituted by 1-4 C₁₋₈-alkoxy, C₁₋₈-alkoxy-C₁₋₈-alkoxy, C₁₋₈-alkoxycarbonyl-(N—C₁₋₈-alkyl)amino, C₁₋₈-alkyl, C₁₋₈-alkylcarbonyl, C₀₋₈-alkylcarbonyl-(N—C₀₋₈-alkyl)amino, C₁₋₈-alkylsulphanyl, C₁₋₈-alkylsulphinyl, C₁₋₈-alkylsulphonyl, aryl-C₀₋₈-alkoxy, aryl, arylamino, aryl-C₀₋₈-alkylsulphonyl, cyano, C₃₋₈-cycloalkoxy, halogen, heterocyclyl, heterocyclyl-C₀₋₈-alkoxy, heterocyclyl-C₀₋₈-alkylamino, heterocyclylcarbonyl, hydroxyl, phosphonyl, optionally N-mono- or N,N-di-C₁₋₈-alkylated amino, optionally N-mono- or N,N-di-C₁₋₈-alkylated carbamoyloxy, optionally N-mono- or N,N-di-C₁₋₈-alkylated sulphamoyl, optionally N-mono-, -di- or -tri-C₁₋₈-alkylated or heterocyclyl-substituted ureido, optionally N-mono- or N,N-di-C₁₋₈-alkylated, N-mono- or N,N-di-arylated or N-mono- or N,N-di-heterocyclyl-C₀₋₈-alkylated amino-C₀₋₈-alkylcarbonyl, oxo or trifluoromethyl; and
R¹ is selected from benzimidazolyl, benzo[1,3]dioxolyl, benzofuranyl, benzoxazolyl, benzothiazolyl, benzo[b]thienyl, quinazolinyl, quinolyl, quinoxalinyl, 2H-chromenyl, carbazolyl, dihydro-2H-benzo[1,4]oxazinyl, dihydro-3H-benzo[1,4]oxazinyl, dihydro-2H-benzo[1,4]thiazinyl, 2,3-dihydroindolyl, dihydro-1H-pyrido[2,3-b][1,4]oxazinyl, imidazo[1,2-a]pyridyl, imidazo[1,5-a]pyridyl, indazolyl, indolyl, isobenzofuranyl, isoquinolyl, [1,5]naphthyridyl, phenyl, phthalazinyl, pyridyl, pyrimidinyl, 1H-pyrrolo[2,3-b]pyridyl, 1H-pyrrolo[2,3-c]pyridyl, 1H-pyrrolo[3,2-b]pyridyl, tetrahydroquinolyl, tetrahydroquinoxalinyl, tetrahydroimidazo[1,2-a]pyridyl, tetrahydroimidazo[1,5-a]pyridyl, tetrahydroisoquinolyl, [1,2,3]triazolo[1,5-a]pyridyl and [1,2,4]triazolo[4,3-a]pyridyl, each of which is substituted by 1-4 acyl-C₁₋₈-alkoxy-C₁₋₈-alkoxy, acyl-C₁₋₈-alkoxy-C₁₋₈-alkyl, (N-acyl)-C₁₋₈-alkoxy-C₁₋₈-alkylamino, C₁₋₈-alkanoyl, C₁₋₈-alkoxy, C₁₋₈-alkoxy-C₁₋₈-alkanoyl, C₁₋₈-alkoxy-C₁₋₈-alkoxy, C₁₋₈-alkoxy-C₁₋₈-alkoxy-C₁₋₈-alkyl, C₁₋₈-alkoxy-C₁₋₈-alkyl, (N—C₁₋₈-alkoxy)-C₁₋₈-alkylaminocarbonyl-C₁₋₈-alkoxy, (N—C₁₋₈-alkoxy)-C₁₋₈-alkylaminocarbonyl-C₁₋₈-alkyl, C₁₋₈-alkoxy-C₁₋₈-alkylcarbamoyl, C₁₋₈-alkoxy-C₁₋₈-alkylcarbonyl, C₁₋₈-alkoxy-C₁₋₈-alkylcarbonylamino, 1-C₁₋₈-alkoxy-C₁₋₈-alkylheterocyclyl, C₁₋₈-alkoxyaminocarbonyl-C₁₋₈-alkoxy, C₁₋₈-alkoxyaminocarbonyl-C₁₋₈-alkyl, C₁₋₈-alkoxycarbonyl, C₁₋₈-alkoxycarbonyl-C₁₋₈-alkoxy, C₁₋₈-alkoxycarbonyl-C₁₋₈-alkyl, C₁₋₈-alkoxycarbonylamino-C₁₋₈-alkoxy, C₁₋₈-alkoxycarbonylamino-C₁₋₈-alkyl, C₁₋₈-alkyl, (N—C₁₋₈-alkyl)-C₁₋₈-alkoxy-C₁₋₈-alkylcarbamoyl, (N—C₁₋₈-alkyl)-C₁₋₈-alkoxy-C₁₋₈-alkylcarbonylamino, (N—C₁₋₈-alkyl)-C₁₋₈-alkoxycarbonylamino, (N—C₁₋₈-alkyl)-C₁₋₈-alkylcarbonylamino-C₁₋₈-alkoxy, (N—C₁₋₈-alkyl)-C₁₋₈-alkylcarbonylamino-C₁₋₈-alkyl, (N—C₁₋₈-alkyl)-C₁₋₈-alkylsulphonylamino-C₁₋₈-alkoxy, (N—C₁₋₈-alkyl)-C₁₋₈-alkylsulphonylamino-C₁₋₈-alkyl, C₁₋₈-alkylamidinyl, C₁₋₈-alkylamino-C₁₋₈-alkoxy, di-C₁₋₈-alkylamino-C₁₋₈-alkoxy, C₁₋₈-alkylamino-C₁₋₈-alkyl, di-C₁₋₈-alkylamino-C₁₋₈-alkyl, C₁₋₈-alkylaminocarbonyl-C₁₋₈-alkoxy, di-C₁₋₈-alkylaminocarbonyl-C₁₋₈-alkoxy, C₁₋₈-alkylaminocarbonyl-C₁₋₈-alkoxy-C₁₋₈-alkyl, C₁₋₈-alkylaminocarbonyl-C₁₋₈-alkyl, di-C₁₋₈-alkylaminocarbonyl-C₁₋₈-alkyl, C₁₋₈-alkylaminocarbonylamino-C₁-alkoxy, C₁₋₈-alkylaminocarbonylamino-C₁₋₈-alkyl, C₁₋₈-alkylcarbonylamino, C₁₋₈-alkylcarbonylamino-C₁₋₈-alkoxy, C₁₋₈-alkylcarbonylamino-C₁₋₈-alkyl, C₁₋₈-alkylcarbonyloxy-C₁₋₈-alkoxy, C₁₋₈-alkylcarbonyloxy-C₁₋₈-alkyl, C₁₋₈-alkylsulphonyl, C₁₋₈-alkylsulphonyl-C₁₋₈-alkoxy, C₁₋₈-alkylsulphonyl-C₁₋₈-alkyl, C₁₋₈-alkylsulphonylamino-C₁₋₈-alkoxy, C₁₋₈-alkylsulphonylamino-C₁₋₈-alkyl, optionally N-mono- or N,N-di-C₁₋₈-alkylated amino, aryl-C₀₋₈-alkoxy, aryl-C₀₋₈-alkyl, optionally N-mono- or N,N-di-C₁₋₈-alkylated carbamoyl-C₀₋₈-alkoxy, optionally N-mono- or N,N-di-C₁₋₈-alkylated carbamoyl-C₀₋₈-alkyl, carboxy-C₁₋₈-alkoxy, carboxy-C₁₋₈-alkoxy-C₁₋₈-alkyl, carboxy-C₁₋₈-alkyl, cyano, cyano-C₁₋₈-alkoxy, cyano-C₁₋₈-alkyl, C₃₋₈-cycloalkyl-C₁₋₈-alkoxy, C₃₋₈-cycloalkyl-C₁₋₈-alkyl, C₃₋₈-cycloalkylcarbonylamino-C₁₋₈-alkoxy, C₃₋₈-cycloalkylcarbonylamino-C₁₋₈-alkyl, O,N-dimethylhydroxylamino-C₁₋₈-alkyl, halogen, halogen-C₁₋₈-alkoxy, halogen-C₁₋₈-alkyl, halogen-aryl, heterocyclyl-C₀₋₈-alkoxy, heterocyclyl-C₀₋₈-alkyl, heterocyclylcarbonyl, hydroxy-C₁₋₈-alkoxy-C₁₋₈-alkoxy, hydroxy-C₁₋₈-alkoxy-C₁₋₈-alkyl, hydroxy-C₁₋₈-alkyl, O-methyloximyl-C₁₋₈-alkyl, oxide or oxo, where, when R¹ is heterocyclyl and contains at least one saturated carbon atom, this heterocyclyl radical may additionally be substituted on a saturated carbon atom by a C₂₋₈-alkylene chain whose two ends are fixed on this saturated carbon atom and thus form a spirocycle, where one CH₂ group of the alkylene chain may be replaced by oxygen.

A further preferred group of compounds of the formula (I), more preferably of the formula (IA), and the salts thereof, preferably the pharmaceutically usable salts thereof, is that of compounds in which

R¹ is aryl or heterocyclyl, each of which is substituted as specified above; and
X is -Alk-, —O-Alk-, -Alk-O—, —O-Alk-O—, -Alk-NR²—, —NR²-Alk-, —C(O)—NR²— or —NR²—C(O)—where Alk is C₁₋₈-alkylene, which may optionally be substituted by halogen; where R² is hydrogen or C₁₋₈-alkyl.

A further preferred group of compounds of the formula (I), and particularly preferably of the formula (IA), and the salts thereof, preferably the pharmaceutically usable salts thereof, are compounds in which

R is C₁₋₈-alkyl, C₀₋₈-alkylcarbonyl-(N—C₀₋₈-alkyl)amino-C₁₋₈-alkyl, C₃₋₈-cycloalkyl-C₁₋₈-alkyl, optionally N-mono- or N,N-di-C₃₋₈-cycloalkyl-C₀₋₈-alkylated or optionally N-mono- or N,N-di-heterocyclyl-C₀₋₈-alkylated carbamoyl-C₀₋₈-alkyl or C₃₋₈-cycloalkyl-C₀₋₈-alkylcarbonyl-(N—C₀₋₈-alkyl)amino-C₁₋₈-alkyl or heterocyclylcarbonyl-C₀₋₈-alkyl, each of which is either unsubstituted or substituted by 1-4 C₁₋₈-alkoxy, C₁₋₈-alkoxy-C₁₋₈-alkoxy, C₁₋₈-alkoxycarbonyl-(N—C₁₋₈-alkyl)amino, C₁₋₈-alkyl, C₁₋₈-alkylcarbonyl, C₀₋₈-alkylcarbonyl-(N—C₁₋₈-alkyl)amino, C₁₋₈-alkylsulphanyl, C₁₋₈-alkylsulphinyl, C₁₋₈-alkylsulphonyl, aryl-C₀₋₈-alkoxy, aryl, arylamino, aryl-C₀₋₈-alkylsulphonyl, cyano, C₃₋₈-cycloalkoxy, halogen, heterocyclyl, heterocyclyl-C₀₋₈-alkoxy, heterocyclyl-C₀₋₈-alkylamino, heterocyclylcarbonyl, hydroxyl, phosphonyl, optionally N-mono- or N,N-di-C₁₋₈-alkylated amino, optionally N-mono- or N,N-di-C₁₋₈-alkylated carbamoyloxy, optionally N-mono- or N,N-di-C₁₋₈-alkylated sulphamoyl, optionally N-mono-, -di- or -tri-C₁₋₈-alkylated or heterocyclyl-substituted ureido, optionally N-mono- or N,N-di-C₁₋₈-alkylated, N-mono- or N,N-di-arylated or N-mono- or N,N-di-heterocyclyl-C₀₋₈-alkylated amino-C₀₋₈-alkylcarbonyl, oxo or trifluoromethyl.
R is particularly preferably C₁₋₈-alkyl, C₀₋₈-alkylcarbonyl-(N—C₀₋₈-alkyl)amino-C₁₋₈-alkyl, C₃₋₈-cycloalkyl-C₁₋₈-alkyl, optionally N-mono- or N,N-di-C₃₋₈-cycloalkyl-C₀₋₈-alkylated or optionally N-mono- or N,N-diheterocyclyl-C₀₋₈-alkylated carbamoyl-C₀₋₈-alkyl, C₃₋₈-cycloalkyl-C₀₋₈-alkylcarbonyl-(N—C₀₋₈-alkyl)amino-C₁₋₈-alkyl or heterocyclylcarbonyl-C₀₋₈-alkyl, each of which is either unsubstituted or substituted by 1-4 C₁₋₈-alkoxy, C₁₋₈-alkoxy-C₁₋₈-alkoxy, C₁₋₈-alkoxycarbonyl-(N—C₁₋₈-alkyl)amino, C₁₋₈-alkyl, C₁₋₈-alkylcarbonyl, C₃₋₈-alkylcarbonyl-(N—C₀₋₈-alkyl)amino, C₁₋₈-alkylsulphonyl, aryl-C₀₋₈-alkoxy, aryl, arylamino, cyano, C₃₋₈-cycloalkoxy, halogen, heterocyclyl, heterocyclyl-C₀₋₈-alkoxy, heterocyclyl-C₀₋₈-alkylamino, heterocyclylcarbonyl, hydroxyl, optionally N-mono- or N,N-di-C₁₋₈-alkylated amino, optionally N-mono- or N,N-di-C₁₋₈-alkylated carbamoyloxy, optionally N-mono- or N,N-di-C₁₋₈-alkylated sulphamoyl, optionally N-mono-, di-, or tri-C₁₋₈-alkylated or heterocyclyl substituted ureido, optionally N-mono- or N,N-di-C₁₋₈-alkylated, N-mono- or N,N-diarylated or N-mono- or N,N-diheterocyclyl-C₀₋₈-alkylated amino-C₀₋₈-alkylcarbonyl, oxo or trifluoromethyl.
R is very particularly preferably C₁₋₈-alkyl, C₀₋₈-alkylcarbonylamino-C₁₋₈-alkyl, C₃₋₈-cycloalkyl-C₁₋₈-alkyl, optionally N-mono-C₃₋₈-cycloalkyl-C₀₋₈-alkylated, optionally N-mono-heterocyclyl-C₀₋₈-alkylated carbamoyl-C₀₋₈-alkyl, C₃₋₈-cycloalkyl-C₀₋₈-alkylcarbonylamino-C₁₋₈-alkyl or heterocyclylcarbonyl-C₀₋₈-alkyl, each of which is either unsubstituted or substituted by 1-2 heterocyclyl or optionally N-mono- or N,N-di-C₁₋C₈₋alkylated, N-mono- or N,N-di-arylated or N-mono- or N,N-di-heterocyclyl-C₀₋₈-alkylated carbamoyl, wherein the heterocyclyl is particularly preferably a saturated heterocyclyl preferably selected from the group comprising tetrahydropyranyl, morpholinyl, piperidinyl, tetrahydrofuranyl, 3-oxa-bicyclo[3.1.0]hexanyl and 6-oxa-spiro[2.5]octanyl which heterocyclyl substituents may be further substituted.

A further preferred group of compounds of the formula (I), and particularly preferably of the formula (IA), and the salts thereof, preferably the pharmaceutically acceptable salts thereof, are compounds in which

R¹ is benzimidazolyl, benzo[1,3]dioxolyl, benzofuranyl, benzoxazolyl, benzothiazolyl, benzo[b]thienyl, quinazolinyl, quinolyl, quinoxalinyl, 2H-chromenyl, carbazolyl, dihydro-2H-benzo[1,4]oxazinyl, dihydro-3H-benzo[1,4]oxazinyl, dihydro-2H-benzo[1,4]thiazinyl, 2,3-dihydroindolyl, dihydro-1H-pyrido[2,3-b][1,4]oxazinyl, imidazo[1,2-a]pyridyl, imidazo[1,5-a]pyridyl, indazolyl, indolyl, isobenzofuranyl, isoquinolyl, [1,5]naphthyridyl, phenyl, phthalazinyl, pyridyl, pyrimidinyl, 1H-pyrrolo[2,3-b]pyridyl, 1H-pyrrolo[2,3-c]pyridyl, 1H-pyrrolo[3,2-b]pyridyl, tetrahydroquinolyl, tetrahydroquinoxalinyl, tetrahydroimidazo[1,2-a]pyridyl, tetrahydroimidazo[1,5-a]pyridyl, tetrahydroisoquinolyl, [1,2,3]triazolo[1,5-a]pyridyl or [1,2,4]triazolo[4,3-a]pyridyl, each of which is substituted by 1-4 C₁₋₈-alkanoyl, C₁₋₈-alkoxy, C₁₋₈-alkoxy-C₁₋₈-alkanoyl, C₁₋₈-alkoxy-C₁₋₈-alkoxy, C₁₋₈-alkoxy-C₁₋₈-alkoxy-C₁₋₈-alkyl, C₁₋₈-alkoxy-C₁₋₈-alkyl, C₁₋₈-alkoxycarbonylamino-C₁₋₈-alkoxy, C₁₋₈-alkoxycarbonylamino-C₁₋₈-alkyl, C₁₋₈-alkyl, C₀₋₈-alkylcarbonylamino-C₁₋₈-alkoxy, C₀₋₈-alkylcarbonylamino-C₁₋₈-alkyl, aryl-C₀₋₈alkoxy, aryl-C₀₋₈-alkyl, cyano, cyano-C₁₋₈-alkoxy, cyano-C₁₋₈-alkyl, halogen, halogen-C₁₋₈-alkoxy, halogen-C₁₋₈-alkyl, halogen-aryl, heterocyclyl-C₀₋₈-alkoxy, heterocyclyl-C₀₋₈-alkyl, oxide or oxo,
where, when R¹ is heterocyclyl and contains at least one saturated carbon atom, this heterocyclyl radical may additionally be substituted on a saturated carbon atom by a C₂₋₈-alkylene chain whose two ends are fixed on this saturated carbon atom and thus form a spirocycle, where one CH₂ group of the alkylene chain may be replaced by oxygen.
R¹ is preferably benzo[1,3]dioxolyl, benzofuranyl, benzoimidazolyl, benzooxazolyl, 2H-chromenyl, carbazolyl, dihydro-2H-benzo[1,4]oxazinyl, dihydro-3H-benzo[1,4]oxazinyl, dihydro-2H-benzo[1,4]thiazinyl, indazolyl, indolyl, isobenzofuranyl, [1,5]naphthyridyl, phenyl, phthalazinyl, pyridyl, pyrimidinyl, 1H-pyrrolo[2,3-b]pyridyl or quinolinyl, each of which is substituted by 1-4 C₁₋₈-alkanoyl, C₁₋₈-alkoxy, C₁₋₈-alkoxy-C₁₋₈-alkanoyl, C₁₋₈-alkoxy-C₁₋₈-alkoxy, C₁₋₈-alkoxy-C₁₋₈-alkoxy-C₁₋₈-alkyl, C₁₋₈-alkoxy-C₁₋₈-alkyl, C₁₋₈-alkoxycarbonylamino-C₁₋₈-alkoxy, C₁₋₈-alkoxycarbonylamino-C₁₋₈-alkyl, C₁₋₈-alkyl, C₀₋₈-alkylcarbonylamino-C₁₋₈-alkoxy, C₀₋₈-alkylcarbonylamino-C₁₋₈-alkyl, aryl-C₀₋₈-alkoxy, aryl-C₀₋₈-alkyl, cyano, cyano-C₁₋₈-alkoxy, cyano-C₁₋₈-alkyl, halogen, halogen-C₁₋₈-alkoxy, halogen-C₁₋₈-alkyl, halogen-aryl, heterocyclyl-C₀₋₈-alkoxy, heterocyclyl-C₀₋₈-alkyl, oxide or oxo,
where, when R¹ is heterocyclyl and contains at least one saturated carbon atom, this heterocyclyl radical may additionally be substituted on a saturated carbon atom by a C₂₋₈-alkylene chain whose two ends are fixed on this saturated carbon atom and thus form a spirocycle, where one CH₂ group of the alkylene chain may be replaced by oxygen.
R¹ is particularly preferably 2H-chromenyl, 3,4-dihydro-2H-benzo[1,4]oxazinyl or carbazolyl, each of which is substituted by 1-4 C₁₋₈-alkoxy, C₁₋₈-alkoxy-C₁₋₈-alkoxy, C₁₋₈-alkoxy-C₁₋₈-alkoxy-C₁₋₈-alkyl, C₁₋₈-alkoxy-C₁₋₈-alkyl, C₁₋₈-alkoxycarbonylamino-C₁₋₈-alkoxy, C₁₋₈-alkoxycarbonylamino-C₁₋₈-alkyl, C₁₋₈-alkyl, C₀₋₈-alkylcarbonylamino-C₁₋₈-alkoxy, C₀₋₈-alkylcarbonylamino-C₁₋₈-alkyl, halogen, halogen-C₁₋₈-alkoxy, halogen-C₁₋₈-alkyl, halogen-aryl or oxo,
where a saturated carbon atom of the heterocyclyl radical may additionally be substituted on the saturated carbon atom by a C₂₋₈-alkylene chain whose two ends are fixed on this saturated carbon atom and thus form a spirocycle, where one CH₂ group of the alkylene chain may be replaced by oxygen.
R¹ is very particularly preferably 3,4-dihydro-2H-benzo[1,4]oxazinyl substituted by 1-4 C₁₋₈-alkoxy, C₁₋₈-alkoxy-C₁₋₈-alkoxy, C₁₋₈-alkoxy-C₁₋₈-alkoxy-C₁₋₈-alkyl, C₁₋₈-alkoxy-C₁₋₈-alkyl, C₁₋₈-alkoxycarbonylamino-C₁₋₈-alkoxy, C₁₋₈-alkoxycarbonylamino-C₁₋₈-alkyl, C₁₋₈-alkyl, C₀₋₈-alkylcarbonylamino-C₁₋₈-alkoxy, C₀₋₈-alkylcarbonylamino-C₁₋₈-alkyl, halogen, halogen-C₁₋₈-alkoxy, halogen-C₁₋₈-alkyl, halogen-aryl or oxo, where a saturated carbon atom of the heterocyclyl radical may additionally be substituted on the saturated carbon atom by a C₂₋₈-alkylene chain whose two ends are fixed on this saturated carbon atom and thus form a spirocycle, where one CH₂ group of the alkylene chain may be replaced by oxygen.

A further preferred group of compounds of the formula (I), and particularly preferably of the formula (IA), and the salts thereof, preferably the pharmaceutically usable salts thereof, are compounds in which

X is —O-Alk- or —O-Alk-O— where Alk is C₁₋₈-alkylene.
X is particularly preferred —O-Alk-, and very particularly preferred —O—CH₂—.

A further preferred group of compounds of the formula (I), and particularly preferably of the formula (IA), and the salts thereof, preferably the pharmaceutically usable salts thereof, are compounds in which

R is C₂₋₈-alkenyl, C₂₋₈-alkynyl, C₁₋₈-alkyl, C₀₋₈-alkylcarbonyl-(N—C₀₋₈-alkyl)amino-C₁₋₈-alkyl, optionally N-mono- or N,N-di-C₁₋₈-alkylated or -arylated amino-C₁₋₈-alkyl, optionally O—C₁₋₈-alkylated carboxyl-C₃₋₈-alkyl, C₃₋₈-cycloalkyl-C₁₋₈-alkyl, optionally N-mono- or N,N-di-C₃₋₈-cycloalkyl-C₀₋₈-alkylated or optionally N-mono- or N,N-di-heterocyclyl-C₀₋₈-alkylated carbamoyl-C₀₋₈-alkyl, C₃₋₈-cycloalkyl-C₀₋₈-alkylcarbonyl-(N—C₀₋₈-alkyl)amino-C₁₋₈-alkyl, arylcarbonyl-(N—C₀₋₈-alkyl)amino-C₁₋₈-alkyl, optionally N-mono- or N,N-di-C₁₋₈-alkylated or -arylated sulphamoyl-C₀₋₈-alkyl, C₁₋₈-alkylsulphonyl-C₀₋₈-alkyl or heterocyclylcarbonyl-C₀₋₈-alkyl, each of which is either un-substituted or substituted by 1-4 C₁₋₈-alkoxy, C₁₋₈-alkoxy-C₁₋₈-alkoxy, C₁₋₈-alkoxycarbonyl-(N—C₁₋₈-alkyl)amino, C₁₋₈-alkyl, C₁₋₈-alkylcarbonyl, C₀₋₈-alkylcarbonyl-(N—C₀₋₈-alkyl)amino, C₁₋₈-alkylsulphanyl, C₁₋₈-alkylsulphinyl, C₁₋₈-alkylsulphonyl, aryl-C₀₋₈-alkoxy, aryl, arylamino, aryl-C₀₋₈-alkylsulphonyl, cyano, C₃₋₈-cycloalkoxy, halogen, heterocyclyl, heterocyclyl-C₀₋₈-alkoxy, heterocyclyl-C₀₋₈-alkylamino, heterocyclylcarbonyl, hydroxyl, phosphonyl, optionally N-mono- or N,N-di-C₁₋₈-alkylated amino, optionally N-mono- or N,N-di-C₁₋₈-alkylated carbamoyloxy, optionally N-mono- or N,N-di-C₁₋₈-alkylated sulphamoyl, optionally N-mono-, -di- or -tri-C₁₋₈-alkylated or heterocyclyl-substituted ureido, optionally N-mono- or N,N-di-C₁₋₈-alkylated, N-mono- or N,N-di-arylated or N-mono- or N,N-di-heterocyclyl-C₀₋₈-alkylated amino-C₀₋₈-alkylcarbonyl, oxo or trifluoromethyl, wherein the heterocyclyl is particularly preferably a saturated heterocyclyl preferably selected from the group comprising tetrahydropyranyl, morpholinyl, piperidinyl, tetrahydrofuranyl, 3-oxa-bicyclo[3.1.0]hexanyl and 6-oxa-spiro[2.5]octanyl which heterocyclyl substituents may be further substituted;
R¹ is 2H-chromenyl, 3,4-dihydro-2H-benzo[1,4]oxazinyl or carbazolyl, each of which is substituted by 1-4 C₁₋₈-alkoxy, C₁₋₈-alkoxy-C₁₋₈-alkoxy, C₁₋₈-alkoxy-C₁₋₈-alkoxy-C₁₋₈-alkyl, C₁₋₈-alkoxy-C₁₋₈-alkyl, C₁₋₈-alkoxycarbonylamino-C₁₋₈-alkoxy, C₁₋₈-alkoxycarbonylamino-C₁₋₈-alkyl, C₁₋₈-alkyl, C₀₋₈-alkylcarbonylamino-C₁₋₈-alkoxy, C₀₋₈-alkylcarbonylamino-C₁₋₈-alkyl, halogen, halogen-C₁₋₈-alkoxy, halogen-C₁₋₈-alkyl, halogen-aryl or oxo,
where a saturated carbon atom of the heterocyclyl radical may additionally be substituted on the saturated carbon atom by a C₂₋₈-alkylene chain whose two ends are fixed on this saturated carbon atom and thus form a spirocycle, where one CH₂ group of the alkylene chain may be replaced by oxygen and

### X is —O—CH₂—.

A further preferred group of compounds of the formula (I), and particularly preferably of the formula (IA), and the salts thereof, preferably the pharmaceutically usable salts thereof, are compounds in which

R is C₂₋₈-alkenyl, C₂₋₈-alkynyl, C₁₋₈-alkyl, C₀₋₈-alkylcarbonyl-(N—C₀₋₈-alkyl)amino-C₁₋₈-alkyl, optionally N-mono- or N,N-di-C₁₋₈-alkylated or -arylated amino-C₁₋₈-alkyl, optionally O—C₁₋₈-alkylated carboxyl-C₀₋₈-alkyl, C₃₋₈-cycloalkyl-C₁₋₈-alkyl, optionally N-mono- or N,N-di-C₁₋₈-cycloalkyl-C₀₋₈-alkylated or optionally N-mono- or N,N-di-heterocyclyl-C₀₋₈-alkylated carbamoyl-C₀₋₈-alkyl, C₃₋₈-cycloalkyl-C₀₋₈-alkylcarbonyl-(N—C₀₋₈-alkyl)amino-C₁₋₈-alkyl, arylcarbonyl-(N—C₀₋₈-alkyl)amino-C₁₋₈-alkyl, optionally N-mono- or N,N-di-C₁₋₈-alkylated or -arylated sulphamoyl-C₀₋₈-alkyl, C₁₋₈-alkylsulphonyl-C₀₋₈-alkyl or heterocyclylcarbonyl-C₀₋₈-alkyl, each of which is either unsubstituted or substituted by 1-4 C₁₋₈-alkoxy, C₁₋₈-alkoxy-C₁₋₈-alkoxy, C₁₋₈-alkoxycarbonyl-(N—C₁₋₈-alkyl)amino, C₁₋₈-alkyl, C₁₋₈-alkylcarbonyl, C₀₋₈-alkylcarbonyl-(N—C₀₋₈-alkyl)amino, C₁₋₈-alkylsulphanyl, C₁₋₈-alkylsulphinyl, C₁₋₈-alkylsulphonyl, aryl-C₀₋₈-alkoxy, aryl, arylamino, aryl-C₀₋₈-alkylsulphonyl, cyano, C₃₋₈-cycloalkoxy, halogen, heterocyclyl, heterocyclyl-C₀₋₈-alkoxy, heterocyclyl-C₀₋₈-alkylamino, heterocyclylcarbonyl, hydroxyl, phosphonyl, optionally N-mono- or N,N-di-C₁₋₈-alkylated amino, optionally N-mono- or N,N-di-C₁₋₈-alkylated carbamoyloxy, optionally N-mono- or N,N-di-C₁₋₈-alkylated sulphamoyl, optionally N-mono-, -di- or -tri-C₁₋₈-alkylated or heterocyclyl-substituted ureido, optionally N-mono- or N,N-di-C₁₋₈-alkylated, N-mono- or N,N-di-arylated or N-mono- or N,N-di-heterocyclyl-C₀₋₈-alkylated amino-C₀₋₈-alkylcarbonyl, oxo or trifluoromethyl;
R¹ is 2H-chromenyl, 3,4-dihydro-2H-benzo[1,4]oxazinyl or carbazolyl, each of which is substituted by 1-4 C₁₋₈-alkoxy, C₁₋₈-alkoxy-C₁₋₈-alkoxy, C₁₋₈-alkoxy-C₁₋₈-alkoxy-C₁₋₈-alkyl, C₁₋₈-alkoxy-C₁₋₈-alkyl, C₁₋₈-alkoxycarbonylamino-C₁₋₈-alkoxy, C₁₋₈-alkoxycarbonylamino-C₁₋₈-alkyl, C₁₋₈-alkyl, C₀₋₈-alkylcarbonylamino-C₁₋₈-alkoxy, C₀₋₈-alkylcarbonylamino-C₁₋₈-alkyl, halogen, halogen-C₁₋₈-alkoxy, halogen-C₁₋₈-alkyl, halogen-aryl or oxo,
where a saturated carbon atom of the heterocyclyl radical may additionally be substituted on the saturated carbon atom by a C₂₋₈-alkylene chain whose two ends are fixed on this saturated carbon atom and thus form a spirocycle, where one CH₂ group of the alkylene chain may be replaced by oxygen and

### X is —O—CH₂—.

A further preferred group of compounds of the formula (I), and particularly preferably of the formula (IA), and the salts thereof, preferably the pharmaceutically usable salts thereof, are compounds in which

R is C₁₋₈-alkyl, C₀₋₈-alkylcarbonylamino-C₁₋₈-alkyl, C₃₋₈-cycloalkyl-C₁₋₈-alkyl, optionally N-mono-C₃₋₈-cycloalkyl-Cm-alkylated, optionally N-mono-heterocyclyl-C₀₋₈-alkylated carbamoyl-C₀₋₈-alkyl, CG-cycloalkyl-C₀₋₈-alkylcarbonylamino-C₁₋₈-alkyl or heterocyclylcarbonyl-C₀₋₈-alkyl, each of which is either unsubstituted or substituted by 1-2 heterocyclyl or optionally N-mono- or N,N-di-C₁-C₈-alkylated, N-mono- or N,N-di-arylated or N-mono- or N,N-di-heterocyclyl-C₀₋₈-alkylated carbamoyl, wherein the heterocyclyl is particularly preferably a saturated heterocyclyl preferably selected from the group comprising tetrahydropyranyl, morpholinyl, piperidinyl, tetrahydrofuranyl, 3-oxa-bicyclo[3.1.0]hexanyl and 6-oxa-spiro[2.5]octanyl which heterocyclyl substituents may be further substituted;
R¹ is 2H-chromenyl, 3,4-dihydro-2H-benzo[1,4]oxazinyl or carbazolyl, each of which is substituted by 1-4 C₁₋₈-alkoxy, C₁₋₈-alkoxy-C₁₋₈-alkoxy, C₁₋₈-alkoxy-C₁₋₈-alkoxy-C₁₋₈-alkyl, C₁₋₈-alkoxy-C₁₋₈-alkyl, C₁₋₈-alkoxycarbonylamino-C₁₋₈-alkoxy, C₁₋₈-alkoxycarbonylamino-C₁₋₈-alkyl, C₁₋₈alkyl, C₀₋₈-alkylcarbonylamino-C₁₋₈-alkoxy, C₀₋₈-alkylcarbonylamino-C₁₋₈-alkyl, halogen, halogen-C₁₋₈-alkoxy, halogen-C₁₋₈-alkyl, halogen-aryl or oxo,
where a saturated carbon atom of the heterocyclyl radical may additionally be substituted on the saturated carbon atom by a C₂₋₈-alkylene chain whose two ends are fixed on this saturated carbon atom and thus form a spirocycle, where one CH₂ group of the alkylene chain may be replaced by oxygen and

### X is —O—CH₂—.

The abovementioned compound groups should not be regarded as closed, but rather it is possible in a sensible manner, for example to replace general by more specific definitions by exchanging parts of these compound groups with one another or with the definitions given above or omitting them. In case a range is indicated, for example 1-4, both endpoints are comprised in the range. The preferences apply equally also to the use of the compounds of the formulae (I) and (IA), and to the pharmaceutical products comprising the compounds of the formula (I) or (IA).

The compounds of the formula (I) may also be prepared in optically pure form. The separation into antipodes can be effected by methods known per se, either preferably at a synthetically early stage by salt formation with an optically active acid, for example (+)- or (−)-mandelic acid, and separation of the diastereomeric salts by fractional crystallization, or preferably at a rather late stage by derivatization with a chiral auxiliary unit, for example (+)- or (−)-camphanoyl chloride, and separation of the diastereomeric products by chromatography and/or crystallization and subsequent cleavage of the bond to the chiral auxiliary. To determine the absolute configuration of the piperidine present, the pure diastereomeric salts and derivatives may be analysed by common spectroscopic-methods, and X-ray spectroscopy on single crystals constitutes a particularly suitable method.

The compounds of the formula (I) or formula (IA) may be prepared in an analogous manner to preparation processes known from the literature. A preferred method of preparing optically pure compounds of the formula (IA) consists in the formation of a piperidine base structure, for example 6(S)-hydroxymethylpiperidin-3(R)-ol [406945-58-2], by reacting an aspartic acid derivative with Meldrum's acid and a subsequent diastereoselective α-hydroxylation according to the exemplary scheme which follows:

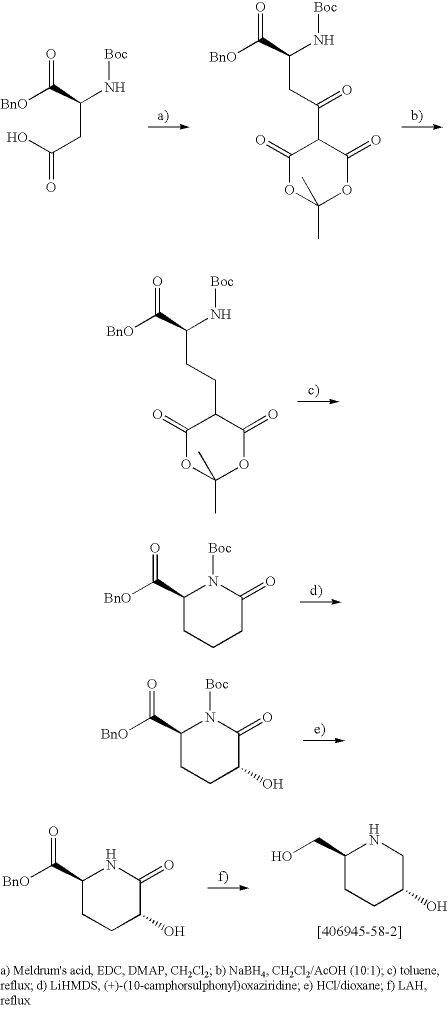

Further details and alternative preparation processes are specified in J. Org. Chem. 67 (2002), 8440-8449 and Lieb. Ann./Recueil (1997), 1267-1272, and literature cited there.

Details of the specific preparation variants can be taken from the examples.

The compounds of the formula (I) or formula (IA) also include those compounds in which one or more atoms are replaced by their stable, non-radioactive isotopes; for example a hydrogen atom by deuterium.

Prodrug derivatives of the compounds described in the present context are derivatives thereof which release the original compound by a chemical or physiological process on in vivo administration. A prodrug can be converted to the original compound, for example, on attainment of a physiological pH or by enzymatic conversion. Prodrug derivatives may, for example, be esters of freely available carboxylic acids, S- and O-acyl derivatives of thiols, alcohols or phenols, where the acyl group is as defined in the present context. Preference is given to pharmaceutically usable ester derivatives which are converted to the original carboxylic acid by solvolysis in physiological medium, for example lower alkyl esters, cycloalkyl esters, lower alkenyl esters, benzyl esters, mono- or disubstituted lower alkyl esters, such as lower ω-(amino, mono- or dialkylamino, carboxyl, lower alkoxycarbonyl)alkyl esters, or such as lower α-(alkanoyloxy, alkoxycarbonyl or dialkylaminocarbonyl)alkyl esters; as such, pivaloyloxymethyl esters and similar esters are conventionally used.

Owing to the close relationship between a free compound, a prodrug derivative and a salt compound, a particular compound in this invention also encompasses its prodrug derivative and salt form, provided that this is possible and appropriate.

The compounds of the formula (I) or of the formula (IA), and the pharmaceutically usable salts thereof, have inhibiting action on the natural enzyme renin. The latter passes from the kidneys into the blood and there brings about the cleavage of angiotensinogen to form the decapeptide angiotensin I which is then cleaved in the lung, the kidneys and other organs to the octapeptide angiotensin II. Angiotensin II increases the blood pressure both directly by arterial constriction and indirectly by the release of the hormone aldosterone which inhibits the release of sodium ions from the adrenal glands, which is associated with a rise in the extracellular liquid volume. This rise can be attributed to the action of angiotensin II itself or of the heptapeptide angiotensin III formed therefrom as a cleavage product. Inhibitors of the enzymatic activity of renin bring about a reduction in the formation of angiotensin I and, as a consequence thereof, the formation of a smaller amount of angiotensin II. The reduced concentration of this active peptide hormone is the immediate cause of the hypotensive action of renin inhibitors which make renin inhibitors suitable for preventing, for retarding the progression of or for treating hypertension, heart failure, glaucoma, myocardial infarction, kidney failure, restenoses or stroke.

One experimental method of detecting the action of renin inhibitors is by means of in vitro tests, in which the reduction of the formation of angiotensin I in different systems (human plasma, purified human renin together with synthetic or natural renin substrate) is measured. One in vitro test which is used is the one according to Nussberger et al. (1987) J. Cardiovascular Pharmacol., Vol. 9, p. 39-44 which follows. This test measures the formation of angiotensin I in human plasma. The amount of angiotensin I formed is determined in a subsequent radioimmunoassay. Which action inhibitors have on the formation of angiotensin I is tested in this system by the addition of different concentrations of these substances. The IC₅₀ refers to that concentration of the particular inhibitor which reduces the formation of angiotensin I by 50%. The compounds of the present invention exhibit IC₅₀ inhibiting actions in the in vitro systems at minimum concentrations of about 10⁻³ to about 10⁻¹⁰ mol/l. Illustrative of the invention, the compounds of examples 25, 26, 42, 52 and 89 inhibit the formation of angiotensin I with IC₅₀ values in the range of about 74-2804·10⁻⁹ mol/l.

In salt-depleted animals, renin inhibitors bring about a decline in blood pressure. Human renin differs from renin of other species. To test inhibitors of human renin, primates (marmosets, Callithrixjacchus) are used, because human renin and primate renin are substantially homologous in the enzymatically active region. One in vivo test which is used is as follows: the test compounds are tested on normotensive marmosets of both genders and having a body weight of about 350 g which are conscious, able to move freely and in their normal cages. Blood pressure and heart rate are measured using a catheter in the descending aorta and recorded radiometrically. The endogenous release of renin is stimulated by the combination of a 1-week low-salt diet with a single intramuscular injection of furosemide (5-(aminosulphonyl)-4-chloro-2-[(2-furanylmethyl)amino]benzoic acid) (5 mg/kg). 16 hours after the injection of furosemide, the test substances are administered either directly into the femoral artery by means of an injection cannula or into the stomach by gavage as a suspension or solution, and their effect on blood pressure and heart rate is evaluated. The compounds of the present invention effectively reduce blood pressure in the in vivo test described at doses of about 0.003 to about 0.3 mg/kg i.v. and at doses of about 0.3 to about 30 mg/kg p.o.

The compounds of the formula (I), or preferably of the formula (IA), and the pharmaceutically usable salts thereof, may find use as medicines, in particular human medicines, for example in the form of pharmaceutical preparations. The pharmaceutical preparations may be administered enterally, such as orally, for example in the form of tablets, coated tablets, sugar-coated tablets, hard and soft gelatin capsules, solutions, emulsions or suspensions, nasally, for example in the form of nasal sprays, rectally, for example in the form of suppositories, or transdermally, for example in the form of ointments or patches. The administration may also be parenteral, such as intramuscular or intravenous, for example in the form of injection solutions.

To produce tablets, coated tablets, sugar-coated tablets and hard gelatin capsules, the compounds of the formula (I), or preferably of the formula (IA), and the pharmaceutically usable salts thereof may be processed with pharmaceutically inert, inorganic or organic excipients. Such excipients used, for example for tablets, coated tablets and hard gelatin capsules, may be lactose, corn starch, or derivatives thereof, talc, stearic acid or salts thereof etc.

Suitable excipients for soft gelatin capsules are, for example, vegetable oils, waxes, fats, semisolid and liquid polyols, etc.

Suitable excipients for preparing solutions and syrups are, for example, water, polyols, sucrose, invert sugar, glucose, etc.

Suitable excipients for injection solutions are, for example, water, alcohols, polyols, glycerol, vegetable oils, bile acids, lecithin, etc.

Suitable excipients for suppositories are, for example, natural or hardened oils, waxes, fats, semisolid or liquid polyols, etc.

The pharmaceutical preparations may additionally also comprise preservatives, solubilizers, viscosity-increasing substances, stabilizers, wetting agents, emulsifiers, sweeteners, colorants, flavourings, salts for altering the osmotic pressure, buffers, coatings or antioxidants. They may also comprise other therapeutically valuable substances.

The present invention further provides the use of the compounds of the formula (I), or preferably of the formula (IA), and the pharmaceutically usable salts thereof, for preventing, for retarding the progression of or for treating hypertension and heart failure, and also glaucoma, myocardial infarction, kidney failure, restenoses and stroke.

The compounds of the formula (I), or preferably of the formula (IA), and the pharmaceutically usable salts thereof may also be administered in combination with one or more agents having cardiovascular action, for example α- and β-blockers such as phentolamine, phenoxybenzamine, prazosin, terazosin, tolazine, atenolol, metoprolol, nadolol, propranolol, timolol, carteolol etc.; vasodilators such as hydralazine, minoxidil, diazoxide, nitroprusside, flosequinan etc.; calcium antagonists such as amrinone, bencyclan, diltiazem, fendiline, flunarizine, nicardipine, nimodipine, perhexylene, verapamil, gallopamil, nifedipine etc.; ACE inhibitors such as cilazapril, captopril, enalapril, lisinopril etc.; potassium activators such as pinacidil; anti-serotoninergics such as ketanserin; thromboxane-synthetase inhibitors; neutral endopeptidase inhibitors (NEP inhibitors); angiotensin II antagonists; and also diuretics such as hydrochlorothiazide, chlorothiazide, acetazolamide, amiloride, bumetanide, benzthiazide, ethacrynic acid, furosemide, indacrinone, metolazone, spironolactone, triamteren, chlorthalidone etc.; sympatholytics such as methyldopa, clonidine, guanabenz, reserpine; and other agents which are suitable for the treatment of hypertension, heart failure or vascular-diseases in humans and animals which are associated with diabetes or renal disorders such as acute or chronic renal failure. Such combinations may be employed separately or in preparations which comprise a plurality of components.

Further substances which can be used in combination with the compounds of the formulae (I) are the compounds of classes (i) to (ix) on page 1 of WO 02/40007 (and also the preferences and examples further listed therein) and the substances specified on pages 20 and 21 of WO 03/027091.

The dose may vary within wide limits and has of course to be adapted to the individual circumstances in each individual case. In general, for oral administration, a daily dose of about 3 mg to about 3 g, preferably about 10 mg to about 1 g, for example about 300 mg, per adult (70 kg), divided into preferably 1-3 individual doses which may, for example, be of equal size, may be appropriate, although the upper limit specified may also be exceeded if this should be found to be appropriate; typically, children receive a lower dose according to their age and body weight.

### EXAMPLES

The examples which follow illustrate the present invention. All temperatures are reported in degrees Celsius, pressures in mbar. Unless stated otherwise, the reactions take place at room temperature. The abbreviation “Rf=xx (A)” means, for example, that the Rf value xx is determined in the solvent system A. The ratio of solvents relative to one another is always reported in parts by volume. Chemical names for end products and intermediates were generated with the aid of the program AutoNom 2000 (automatic nomenclature). The absolute configuration of all compounds is given. In cases where the stereochemical assignment could not be determined with certainty, the opposite configuration of the stereocenter is given in parenthesis.

HPLC gradients:
A) on Hypersil BDS C-18 (5 μm); column: 4×125 mm
I. 90% water*/10% acetonitrile* to 0% water*/100% iacetonitrile* in 5 minutes+2.5 minutes (1.5 ml/min)
II 95% water*/5% acetonitrile* to 0% water*/100% acetonitrile* in 40 minutes (0.8 ml/min)
   * contains 0.1% trifluoroacetic acid
   B) on Supelco Discovery HS C18 (3 μm) (#569250-U); column: 4.6×50 mm
III 90% water*/10% acetonitrile* to 10% water*/90% acetonitrile* in 5 minutes, then to 1% water*/99% acetonitrile* in 1 minute (2.2 ml/min)
   * contains 0.05% trifluoroacetic acid

The following abbreviations are used:
Rf ratio of distance traveled by a substance to separation of the eluent front from the start point in thin-layer chromatography
Rt retention time of a substance in HPLC (in minutes)
m.p. melting point (temperature)

### General Method A: (N-Tos Deprotection)

0.44 mmol of sodium dihydrogenphosphate and 0.90 mmol of sodium amalgam (10% Na) are added successively at room temperature to a solution of 0.09 mmol of “tosylamide” in 10 ml of methanol. The reaction mixture is left to stir for 2-18 hours, diluted with water and extracted with ethyl acetate. The organic phase is removed and washed with brine, dried over sodium sulphate and concentrated by evaporation. The title compound is obtained from the residue by means of flash chromatography (SiO₂ 60F).

### General Method B: (Amide Coupling I)

5.0 mmol of triethylamine and 1.2 mmol of tripropylphosphonic anhydride [68957-94-8] (50% in ethyl acetate) are added at room temperature to a solution of 1.0 mmol of “acid” and 1.2 mmol of “amine” in 20 ml of dichloromethane. The reaction mixture is stirred at room temperature for 1-3 hours and then diluted with dichloromethane, and washed with 1M HCl and finally with brine. The combined organic phases are dried over sodium sulphate and concentrated by evaporation. The title compound is obtained from the residue by means of flash chromatography (SiO₂ 60F).

### General Method C: (Nitrile Hydrolysis)

A suspension of 3.5 mmol of “nitrile” in 55 ml of ethanol and 55 ml of 2M NaOH is stirred at 80° C. for 21 hours. Subsequently, the mixture is concentrated and the residue is adjusted to pH 2-3 with 1M HCl. The mixture is extracted with ethyl acetate (2×). The residue is diluted with dichloromethane, and washed with 1M HCl and finally with brine. The combined organic phases are dried over sodium sulphate and concentrated by evaporation. The crude title compound is obtained from the residue.

### General Method D: (Substitution of Mesylate by Nitrile)

A solution of 0.5 mmol of “mesylate” and 5.5 mmol of sodium cyanide in 3 ml of dimethyl sulphoxide is stirred at 60° C. for 20 hours. Subsequently, the mixture is diluted with ethyl acetate and washed with brine. The aqueous phase is extracted with ethyl acetate (2×). The combined organic phases are dried with sodium sulphate and concentrated by evaporation. The crude title compound is obtained from the residue.

### General Method E: (Alcohol Mesylation)

5 mmol of triethylamine and 2 mmol of methanesulphonyl chloride are added gradually at 0° C. to a solution of 1 mmol of “alcohol” in 10 ml of dichloromethane. The mixture is stirred at 0° C. for one hour, diluted with dichloromethane and washed with 1M HCl. The organic phase is dried over sodium sulphate and concentrated by evaporation. The crude title compound is obtained from the residue.

General Method F (Lactam Reduction with BH₃)

2-4 mmol of a solution of borane-tetrahydrofuran complex (1M in tetrahydrofuran) are added at room temperature to a solution of 1.0 mmol of “lactam” in 800 ml of tetrahydrofuran. The reaction mixture is heated to 50° C. for 2-8 hours, admixed with 10 ml of methanol and concentrated. The title compound is obtained from the residue by means of flash chromatography (SiO₂ 60F).

### General Method G: (O-TIPS Deprotection)

300 mmol of tetrabutylammonium fluoride (1M in tetrahydrofuran) are added to a solution of 160 mmol of “TIPS ether” in 1 l of tetrahydrofuran. The mixture is stirred at room temperature for 18 hours, and the reaction mixture is diluted with brine and extracted with tert-butyl methyl ether (2×). The combined organic phases are dried over sodium sulphate and concentrated by evaporation. The title compound is obtained from the residue by means of flash chromatography (SiO₂ 60F).

### General Method H: (Alcohol-Halomethylaryl Coupling)

A solution of 235 mmol of “alcohol”, 235 mmol of “halomethylaryl” and 282 mmol of tetrabutylammonium iodide in 800 ml of N,N-dimethylformamide is stirred at room temperature for 15 minutes. 239 mmol of sodium hydride (60% dispersion in oil) are added in portions, then the mixture is stirred at room temperature for 3 hours. Ice-water is added and the mixture is extracted with dichloromethane (2×). The combined organic phases are washed with brine, dried over sodium sulphate and concentrated by evaporation. The title compound is obtained from the residue by means of flash chromatography (SiO₂ 60F).

### General Method I: (O-TIPS Protection)

1.185 mol of imidazole and 261 mmol of triisopropylsilyl chloride are added to a solution of 238 mmol of “alcohol” in 1.7 l of N,N-dimethylformamide. The mixture is stirred at room temperature for 18 hours. Subsequently, the mixture is concentrated and the residue is admixed with 1M HCl and water. The mixture is extracted with tert-butyl methyl ether (2×). The combined organic phases are dried over sodium sulphate and concentrated by evaporation. The title compound is obtained from the residue by means of flash chromatography (SiO₂ 60F).

### General Method J: (N-Tosylation)

12.43 mmol of 4-toluenesulphonyl chloride are added to a mixture of 11.3 mmol of “amine” in 200 ml of ethyl acetate and 200 ml of 2M Na₂CO₃ solution. The reaction mixture is stirred at room temperature for 17 hours. The phases are separated and the aqueous phase is extracted with ethyl acetate (2×). The combined organic phases are dried over sodium sulphate and concentrated by evaporation. The title compound is obtained from the residue by means of flash chromatography (SiO₂ 60F).

### General Method K: (Bromination)

60 mmol of bromotrimethylsilane are slowly added dropwise at 20-25° C. to a solution of 40 mmol of “benzyl alcohol” in 125 ml of chloroform. After the addition, the reaction mixture is concentrated by evaporation. The title compound is obtained from the residue by means of flash chromatography (SiO₂ 60F).

General Method L: (Acid Reduction with BH₃)

9.4 mmol of a solution of borane-tetrahydrofuran complex (1M in tetrahydrofuran) are added at room temperature to a solution of 4.7 mmol of “acid” in 50 ml of tetrahydrofuran. The reaction mixture is heated to 50° C. for 17 hours, admixed with 10 ml of methanol and concentrated. The title compound is obtained from the residue by means of flash chromatography (SiO₂ 60F).

### General Method M: (Alcohol Alkylation)

239 mmol of sodium hydride (60% dispersion in oil) are added in portions at room temperature to a solution of 235 mmol of “alcohol” in 800 ml of N,N-dimethylformamide; the mixture is stirred at room temperature for 1 hour. Subsequently, 235 mmol of “haloalkyl” and, if appropriate, 47 mmol of tetrabutylammonium iodide are added and the mixture is stirred at room temperature for a further 3 hours. Ice-water is added and the mixture is extracted with dichloromethane (2×). The combined organic phases are washed with brine, dried over sodium sulphate and concentrated by evaporation. The title compound is obtained from the residue by means of flash chromatography (SiO₂ 60F).

### General Method N: (Amide Coupling II)

98 mmol of triethylamine and 21.6 mmol of “acid chloride” are added to a solution of 19.6 mmol of “amine” in 300 ml of dichloromethane. The reaction mixture is stirred at room temperature for 1 hour and diluted with water and dichloromethane. The organic phase is dried over sodium sulphate and concentrated by evaporation. The title compound is obtained from the residue by means of flash chromatography (SiO₂ 60F).

### General Method O: (Azide Reduction)

0.3 mmol of Pd/C (10%) is added to a solution of 26.2 mmol of “azide” in 1.5 l of methanol. The mixture is hydrogenated up to complete conversion under a hydrogen atmosphere at standard pressure, then the mixture is filtered from the catalyst and concentrated by evaporation. The crude title compound is obtained from the residue.

### General Method P: (Substitution of Mesylate by Azide)

A solution of 0.5 mmol of “mesylate” and 5.5 mmol of sodium azide in 5 ml of dimethyl sulphoxide is stirred at room temperature for 20 hours. Subsequently, the mixture is diluted with water and tert-butyl methyl ether and washed with brine. The aqueous phase is extracted with tert-butyl methyl ether (2×). The combined organic phases are dried over sodium sulphate and concentrated by evaporation. The crude title compound is obtained from the residue.

### General Method Q: (Amide Alkylation)

246 mmol of sodium hydride (60% dispersion in oil) are added in portions at room temperature to a solution of 123 mmol of “primary amide” in 1 l of N,N-dimethylformamide; the mixture is stirred at room temperature for 1 hour. Subsequently, 1.59 mol of “haloalkyl” are added and the mixture is stirred at room temperature for a further 3 hours. 1M NaHCO₃ is added and the mixture is extracted with tert-butyl methyl ether (2×). The combined organic phases are washed with brine, dried over sodium sulphate and concentrated by evaporation. The title compound is obtained from the residue by means of flash chromatography (SiO₂ 60F).

### General Method R: (Substitution of Mesylate by Alkoxide)

28 mmol of sodium hydride (60% dispersion in oil) and 56 mmol of 3-methoxyphenol [150-19-6] are added at room temperature to a solution of 14 mmol of “mesylate” in 500 ml of N,N-dimethylformamide; the mixture is stirred at 90° C. for 8 hours. Subsequently, the mixture is cooled to room temperature. Water is added and the mixture is extracted with ethyl acetate/tetrahydrofuran (10:1) (3×). The combined organic phases are washed with brine, dried over sodium sulphate and concentrated by evaporation. The title compound is obtained from the residue by means of flash chromatography (SiO₂ 60F).

General Method S: (Carbonyl Reduction with BH₃)

2 mmol of a solution of borane-tetrahydrofuran complex (1M in tetrahydrofuran) are added at room temperature to a solution of 1.0 mmol of “carbonyl” in 10 ml of tetrahydrofuran. The reaction mixture is stirred at room temperature for 2 hours, admixed with 10 ml of methanol and concentrated. The two diastereomeric title compounds are obtained from the residue by means of flash chromatography (SiO₂ 60F).

### General Method T: (Alkylmagnesium Halide Addition to the Weinreb Amide)

1.705 mmol of a solution of “alkylmagnesium halide” are added at 0° C. to a solution of 0.34 mmol of “Weinreb amide” in 3 ml of tetrahydrofuran. The reaction mixture is stirred at 0° C. for 1.5 hours, quenched with 1M KHSO₄ and extracted with tert-butyl methyl ether (2×). The combined organic phases are dried over sodium sulphate and concentrated by evaporation. The title compound is obtained from the residue by means of flash chromatography (SiO₂ 60F).

### General Method U: (Substitution of Mesylate by Thiolate)

A solution of 0.5 mmol of “mesylate” and 2 mmol of “sodium thiolate” in 5 ml of dimethyl sulphoxide is stirred at 70° C. for 20 hours. The mixture is diluted with dichloromethane and 1M NaHCO₃ and the phases are separated. The aqueous phase is extracted with dichloromethane (2×). The combined organic phases are dried with sodium sulphate and concentrated by evaporation. The title compound is obtained from the residue by means of flash chromatography (SiO₂ 60F).

### General Method V: (Reductive Amination)

A solution of 1.57 mmol of “ketone”, 3.14 mmol of “amine”, 4.71 mmol of sodium cyanoborohydride and 1.57 mmol of acetic acid in 50 ml of ethanol is stirred at room temperature for 19 hours. The mixture is diluted with dichloromethane and 1M NaOH, and the phases are separated. The aqueous phase is extracted with dichloromethane (2×). The combined organic phases are washed with brine, dried over sodium sulphate and concentrated by evaporation. The crude title compound is obtained from the residue.

### General Method W: (Aryl Halide Formylation)

19.19 mmol of butyllithium (1.6M in hexane) are added at −78° C. to a solution of 17.445 mmol of “aryl halide” in 50 ml of tetrahydrofuran. The reaction mixture is stirred at −78° C. for 30 minutes and quenched with 34.89 mmol of N,N-dimethylformamide. The mixture is stirred at −78° C. for a further one hour, and the reaction is stopped at this temperature by adding 1M HCl. The mixture is warmed to room temperature, neutralized with 1M NaOH and extracted with tert-butyl methyl ether (3×). The combined organic phases are dried over sodium sulphate and concentrated by evaporation. The title compound is obtained from the residue by means of flash chromatography (SiO₂ 60F).

### General Method X: (Dimethylcarbamic Acid Coupling)

8.7 mmol of sodium hydride (60% dispersion in oil) are added in portions at room temperature to a solution of 1.45 mmol of “alcohol” in 10 ml of tetrahydrofuran; the mixture is stirred at room temperature for 30 minutes. Subsequently, 5.08 mmol of dimethylcarbamoyl chloride are added and the mixture is stirred at room temperature for a further 3 hours. Ice-water is added and the mixture is extracted with dichloromethane (2×). The combined organic phases are washed with brine, dried over sodium sulphate and concentrated by evaporation. The title compound is obtained from the residue by means of flash chromatography (SiO₂ 60F).

### General Method Y: (Chlorination)

The solution of 40 mmol of “benzyl alcohol” in 6.40 ml of pyridine and 100 ml of dichloromethane is added dropwise slowly at 0-5° C. to the precooled solution of 7.65 ml of thionyl chloride in 20 ml of dichloromethane. The reaction mixture is stirred at 0° C. and then at room temperature for one hour each and then poured into 200 ml of ice-water. The mixture is extracted with dichloromethane (2×200 ml). The organic phases are washed successively with 1M aqueous sodium hydrogencarbonate solution (2×200 ml) and brine, dried over sodium sulphate and concentrated by evaporation. The title compound is obtained from the residue by means of flash chromatography (SiO₂ 60F).

### Example 1

### N—((R(or S))-2-{(2S,5R)-5-[4-(3-Methoxypropyl)-3,4-dihydro-2H-benzo[1,4]oxazin-6-ylmethoxy]piperidin-2-yl}-1-methylethyl)-2,2-dimethylbropionamide

Analogously to Method A, N-{(R(or S))-2-[(2S,5R)-5-[4-(3-methoxypropyl)-3,4-dihydro-2H-benzo[1,4]oxazin-6-ylmethoxy]-1-(toluene-4-sulphonyl)piperidin-2-yl]-1-methylethyl}-2,2-dimethylpropionamide is reacted. The title compound is identified based on the Rf value.

The starting materials are prepared as follows:

### a) N—{(R(or S))-2-[(2S,5R)-5-[4-(3-Methoxypropyl)-3,4-dihydro-2H-benzo[1,4]oxazin-6-ylmethoxy]-1-(toluene-4-sulphonyl)piperidin-2-yl]-1-methylethyl}-2,2-dimethylpropionamide

Analogously to Method N, (R(or S))-2-[(2S,5R)-5-[4-(3-methoxypropyl)-3,4-dihydro-2H-benzo[1,4]oxazin-6-ylmethoxy]-1-(toluene-4-sulphonyl)piperidin-2-yl]-1-methylethylamine and 2,2-dimethylpropionyl chloride are reacted. The title compound is identified based on the Rf value.

### b) (R(or S))-2-[(2S,5R)-5-[4-(3-Methoxypropyl)-3,4-dihydro-2H-benzo[1,4]oxazin-6-ylmethoxy]-1-(toluene-4-sulphonyl)piperidin-2-yl]-1-methylethylamine

Analogously to Method O, 6-[(3R,6S)-6-((R(or S))-2-azidopropyl)-1-(toluene-4-sulphonyl)piperidin-3-yloxymethyl]-4-(3-methoxypropyl)-3,4-dihydro-2H-benzo[1,4]oxazine is reacted. The title compound is identified based on the Rf value.

### c) 6-[(3R,6S)-6-((R(or S))-2-Azidopropyl)-1-(toluene-4-sulphonyl)piperidin-3-yloxymethyl]-4-(3-methoxypropyl)-3,4-dihydro-2H-benzo[1,4]oxazine

A solution of 2.11 mmol of (S(or R))-2-[(2S,5R)-5-[4-(3-methoxypropyl)-3,4-dihydro-2H-benzo[1,4]oxazin-6-ylmethoxy]-1-(toluene-4-sulphonyl)piperidin-2-yl]-1-methylethyl methanesulphonate in 5 ml of 1,3-dimethyltetrahydropyrimidin-2-one (DMPU) is admixed with 21.1 mmol of sodium azide, and then the reaction mixture is heated to 80° C. After 4 hours, the reaction mixture is cooled to room temperature, diluted with tert-butyl methyl ether, filtered through a small amount of silica gel and concentrated by evaporation. The title compound is identified from the residue based on the Rf value by means of flash chromatography (SiO₂ 60F).

### d) (S(or R))-2-[(2S,5R)-5-[4-(3-Methoxypropyl)-3,4-dihydro-2H-benzo[1,4]oxazin-6-ylmethoxy]-1-(toluene-4-sulphonyl)piperidin-2-yl]-1-methylethyl methanesulphonate

Analogously to Method E, (S(or R))-1-[(2S,5R)-5-[4-(3-methoxypropyl)-3,4-dihydro-2H-benzo[1,4]oxazin-6-ylmethoxy]-1-(toluene-4-sulphonyl)piperidin-2-yl]propan-2-ol is reacted. The title compound is identified based on the Rf value.

### e) (i) (R(or S))-1-[(2S,5R)-5-[4-(3-Methoxypropyl)-3,4-dihydro-2H-benzo[1,4]oxazin-6-ylmethoxy]-1-(toluene-4-sulphonyl)piperidin-2-yl]propan-2-ol and

### (ii) (S(or R))-1-[(2S,5R)-5-[4-(3-Methoxypropyl)-3,4-dihydro-2H-benzo[1,4]oxazin-6-ylmethoxy]-1-(toluene-4-sulphonyl)piperidin-2-yl]propan-2-ol

A solution of 2.76 mmol of [(2S,5R)-5-[4-(3-methoxypropyl)-3,4-dihydro-2H-benzo[1,4]oxazin-6-ylmethoxy]-1-(toluene-4-sulphonyl)piperidin-2-yl]acetaldehyde in 15 ml of tetrahydrofuran at 0° C. is admixed with 5.52 mmol of methylmagnesium bromide (3M in diethyl ether) and then the reaction mixture is warmed to room temperature. After 1 hour, the reaction mixture is poured onto 1N potassium bisulphate solution and extracted with ethyl acetate (3×)—the combined organic phases are washed with brine, dried over sodium sulphate and concentrated by evaporation. The title compounds are identified from the residue based on the Rf values by means of flash chromatography (SiO₂ 60F).

### f) [(2S,5R)-5-[4-(3-Methoxypropyl)-3,4-dihydro-2H-benzo[1,4]oxazin-6-ylmethoxy]-1-(toluene-4-sulphonyl)piperidin-2-yl]acetaldehyde

8.60 mmol of diisobutylaluminium hydride (1M in dichloromethane) is added dropwise at −30° C. to a solution of 5.06 mmol of [(2S,5R)-5-[4-(3-methoxypropyl)-3,4-dihydro-2H-benzo[1,4]oxazin-6-ylmethoxy]-1-(toluene-4-sulphonyl)piperidin-2-yl]acetonitrile in 100 ml of dichloromethane. After 2 hours, the reaction mixture is quenched with 1N HCl. The organic phase is extracted with water—the aqueous phases are each extracted with dichloromethane. The combined organic phases are dried over sodium sulphate and concentrated by evaporation. The crude title compound is obtained from the residue as a brown resin. Rt=4.76 (gradient I).

### g) [(2S,5R)-5-[4-(3-Methoxypropyl)-3,4-dihydro-2H-benzo[1,4]oxazin-6-ylmethoxy]-1-(toluene-4-sulphonyl)piperidin-2-yl]acetonitrile

Analogously to Method D, 10.6 mmol of (2S,5R)-5-[4-(3-methoxypropyl)-3,4-dihydro-2H-benzo[1,4]oxazin-6-ylmethoxy]-1-(toluene-4-sulphonyl)piperidin-2-ylmethyl methanesulphonate are reacted. The title compound is obtained as a yellow oil. Rf=0.38 (2:1 EtOAc-heptane); Rt=4.85 (gradient I).

### h) (2S,5R)-5-[4-(3-Methoxypropyl)-3,4-dihydro-2H-benzo[1,4]oxazin-6-ylmethoxy]-1-(toluene-4-sulphonyl)piperidin-2-ylmethyl methanesulphonate

Analogously to Method E, 9.91 mmol of [(2S,5R)-5-[4-(3-methoxypropyl)-3,4-dihydro-2H-benzo[1,4]oxazin-6-ylmethoxy]-1-(toluene-4-sulphonyl)piperidin-2-yl]methanol are reacted. The crude title compound is obtained as a brown oil. Rf=0.13 (1:1 EtOAc-heptane); Rt=4.83 (gradient I).

### i) [(2S,5R)-5-[4-(3-Methoxypropyl)-3,4-dihydro-2H-benzo[1,4]oxazin-6-ylmethoxy]-1-(toluene-4-sulphonyl)piperidin-2-yl]methanol

Analogously to Method G, 6.66 mmol of 4-(3-methoxypropyl)-6-[(3R,6S)-1-(toluene-4-sulphonyl)-6-triisopropylsilanyloxymethylpiperidin-3-yloxymethyl]-3,4-dihydro-2H-benzo[1,4]oxazine are reacted. The title compound is obtained as a yellowish oil. Rf=0.35 (EtOAc); Rt=4.42 (gradient I).

### j) 4-(3-Methoxypropyl)-6-[(3R,6S)-1-(toluene-4-sulphonyl)-6-triisopropylsilanyloxymethylpiperidin-3-yloxymethyl]-3,4-dihydro-2H-benzo[1,4]oxazine

Analogously to Method F, 6.73 mmol of 4-(3-methoxypropyl)-6-[(3R,6S)-1-(toluene-4-sulphonyl)-6-triisopropylsilanyloxymethylpiperidin-3-yloxymethyl]-4H-benzo[1,4]oxazin-3-one are reacted. The crude title compound is obtained as a yellowish oil. Rf=0.25 (1:3 EtOAc-heptane); Rt=6.30 (gradient I).

### k) 4-(3-Methoxypropyl)-6-[(3R,6S)-1-(toluene-4-sulphonyl)-6-triisopropylsilanyloxymethylpiperidin-3-yloxymethyl]-4H-benzo[1,4]oxazin-3-one

Analogously to Method H, 11 mmol of (3R,6S)-1-(toluene-4-sulphonyl)-6-triisopropylsilanyloxymethylpiperidin-3-ol and 11 mmol of 6-chloromethyl-4-(3-methoxypropyl)-4H-benzo[1,4]oxazin-3-one [857272-02-7] are reacted. The title compound is obtained as a yellow oil. Rf=0.25 (1:3 EtOAc-heptane); Rt=6.84 (gradient I).

### l) (3R,6S)-1-(Toluene-4-sulphonyl)-6-triisopropylsilanyloxymethylpiperidin-3-ol

A solution of 0.018 mol of (3R,6S)-6-hydroxymethyl-1-(toluene-4-sulphonyl)piperidin-3-ol in 85 ml of N,N-dimethylformamide at room temperature is admixed with 0.09 mol of imidazole and 0.02 mol of triisopropylsilyl chloride. Further triisopropylsilyl chloride (1.8, 9 and 4.5 mmol) is added after 20, 23 and 39 hours. After a total of 42 hours, the reaction mixture is diluted with water and extracted with tert-butyl methyl ether (2×). The combined organic phases are washed with brine, dried over sodium sulphate and concentrated by evaporation. The title compound is obtained from the residue by means of flash chromatography (SiO₂ 60F) as a yellow-orange oil. Rt=5.70 (gradient III).

### m) (3R,6S)-6-Hydroxymethyl-1-(toluene-4-sulphonyl)piperidin-3-ol

An aqueous solution (28.5 ml) comprising approx. 6 mmol of (3R,6S)-6-hydroxymethylpiperidin-3-ol from the preceding stage is brought to pH 8 with 0.5 ml of conc. HCl and then 39.6 mmol of sodium carbonate are added. A solution of 9 mmol of p-toluenesulphonyl chloride in 30 ml of tetrahydrofuran is added dropwise at room temperature and then the triphasic mixture is heated to 70° C. Further p-toluenesulphonyl chloride (3 mmol) is added after 16 and 18 hours. After a total of 22 hours, the reaction mixture is cooled to 40° C. and concentrated by evaporation. The aqueous residue is extracted with ethyl acetate (2×)—the combined organic phases are washed successively with 10% sodium carbonate solution (2×) and brine, dried over sodium sulphate and concentrated by evaporation. The title compound is obtained from the residue by means of flash chromatography (SiO₂ 60F) as a brown oil. Rf=0.60 (dichloromethane-methanol=4:1+1% of 25% conc. ammonia); Rt=1.82 (gradient III).

### n) (3R,6S)-6-Hydroxymethylpiperidin-3-ol

2.5 mmol of lithium aluminium hydride are suspended in tetrahydrofuran and a solution of 1.0 mmol of benzyl (2S,5R)-5-hydroxy-6-oxopiperidine-2-carboxylate in tetrahydrofuran is added dropwise at 66° C. After the addition has ended, the mixture is stirred at 66° C. for another 2 hours. The suspension is cooled to 0° C., quenched with water and acidified with 3N HCl. The tetrahydrofuran is removed on a rotary evaporator. The yellow suspension is taken up in ethyl acetate and the organic phase is washed with 2N HCl. The combined water phases are brought to pH>11 with 30% NaOH, the resulting suspension is filtered and the precipitate is washed with water. The yellow solution comprising the crude title compound is used directly in the next stage. Rf=0.20 (dichloromethane-methanol=4:1+1% of 25% conc. ammonia).

### o) Benzyl (2S,5R)-5-hydroxy-6-oxopiperidine-2-carboxylate

A solution of 0.017 mol of benzyl tert-butyl (2S,5R)-5-hydroxy-6-oxopiperidine-1,2-dicarboxylate [480460-75-1] in 48 ml of dichloromethane at room temperature is admixed with 12 ml of trifluoroacetic acid and the reaction mixture is stirred overnight. The reaction mixture is added dropwise to saturated sodium hydrogencarbonate solution and then extracted with dichloromethane (2×). The combined organic phases are dried over sodium sulphate and concentrated by evaporation. The title compound is obtained from the residue by means of flash chromatography (SiO₂ 60F) as a white-yellow crystalline solid. Rf=0.50 (EtOAc); Rt=3.17 (gradient III).

The following compound is prepared in an analogous manner by the process described in Example 1:

### 2 N—((S(or R))-2-{(2S,5R)-5-[4-(3-Methoxypropyl)-3,4-dihydro-2H-benzo[1,4]oxazin-6-ylmethoxy]piperidin-2-yl}-1-methyl-ethyl)-2,2-dimethylpropionamide

Starting from (R(or S))-1-[(2S,5R)-5-[4-(3-methoxypropyl)-3,4-dihydro-2H-benzo[1,4]oxazin-6-ylmethoxy]-1-(toluene-4-sulphonyl)piperidin-2-yl]propan-2-ol (Example 1e(i)).

### Example 15

### 6-{(3R,6S)-6-[2-(2-Methoxyethoxy)-2-methylpropyl]piperidin-3-yloxymethyl}-4-(3-methoxypropyl)-3,4-dihydro-2H-benzo[1,4]oxazine

Analogously to Method A, 6-[(3R,6S)-6-[2-(2-methoxyethoxy)-2-methylpropyl]-1-(toluene-4-sulphonyl)piperidin-3-yloxymethyl]-4-(3-methoxypropyl)-3,4-dihydro-2H-benzo[1,4]oxazine is reacted. The title compound is identified based on the Rf value.

The starting materials are prepared as follows:

### a) 6-[(3R,6S)-6-[2-(2-Methoxyethoxy)-2-methylpropyl]-1-(toluene-4-sulphonyl)piperidin-3-yloxymethyl]-4-(3-methoxypropyl)-3,4-dihydro-2H-benzo[1,4]oxazine

A solution of 0.54 mmol of 1-[(2S,5R)-5-[4-(3-methoxypropyl)-3,4-dihydro-2H-benzo[1,4]oxazin-6-ylmethoxy]-1-(toluene-4-sulphonyl)piperidin-2-yl]-2-methylpropan-2-ol in 2 ml of N,N-dimethylformamide at room temperature is admixed with 2.68 mmol of sodium hydride (60% dispersion in oil) and the reaction mixture is stirred for 1 hour. The reaction mixture is then admixed with 8.02 mmol of 1-chloro-2-methoxyethane and 0.11 mmol of tetrabutylammonium iodide and heated to 90° C. After 19 hours, the reaction mixture is cooled to room temperature, admixed cautiously with water and extracted with ethyl acetate (2×)—the combined organic phases are washed with brine, dried over sodium sulphate and concentrated by evaporation. The title compound is identified from the residue by means of flash chromatography (SiO₂ 60F) based on the Rf value.

### b) 1-[(2S,5R)-5-[4-(3-Methoxypropyl)-3,4-dihydro-2H-benzo[1,4]oxazin-6-ylmethoxy]-1-(toluene-4-sulphonyl)piperidin-2-yl]-2-methylpropan-2-ol

A solution of 2.45 mmol of 1-[(2S,5R)-5-[4-(3-methoxypropyl)-3,4-dihydro-2H-benzo[1,4]oxazin-6-ylmethoxy]-1-(toluene-4-sulphonyl)piperidin-2-yl]propan-2-one in 10 ml of tetrahydrofuran at room temperature is admixed with 2.45 mmol of methylmagnesium chloride (3M in tetrahydrofuran). After 4 hours, the reaction mixture is poured onto 1N potassium bisulphate solution and extracted with ethyl acetate (2×)—the combined organic phases are dried with sodium sulphate and concentrated by evaporation. The title compound is identified from the residue by means of flash chromatography (SiO₂ 60F) based on the Rf value.

### c) 1-[(2S,5R)-5-[4-(3-Methoxypropyl)-3,4-dihydro-2H-benzo[1,4]oxazin-6-ylmethoxy]-1-(toluene-4-sulphonyl)piperidin-2-yl]propan-2-one

A solution of 5.0 mmol of (S,R)-1-[(2S,5R)-5-[4-(3-methoxypropyl)-3,4-dihydro-2H-benzo[1,4]oxazin-6-ylmethoxy]-1-(toluene-4-sulphonyl)piperidin-2-yl]propan-2-ol (Example 1e(i,ii)) in 10 ml of dimethyl sulphoxide and 50 ml of dichloromethane at 0° C. is admixed with 25.0 mmol of triethylamine and 16.5 mmol of pyridine-sulphur trioxide complex (in portions over 5 minutes) and then warmed slowly to room temperature. After 3 hours, the reaction mixture is poured onto ice-water, acidified to pH 2.5 with 1N potassium bisulphate solution and extracted with diethyl ether (2×). The combined organic phases are washed successively with water and 5% sodium hydrogencarbonate solution, dried over sodium sulphate and concentrated by evaporation. The title compound is identified from the residue by means of flash chromatography (SiO₂ 60F) based on the Rf value.

### Example 16

### 2-{(2S,5R)-5-[4-(3-Methoxypropyl)-3,4-dihydro-2H-benzo[1,4]oxazin-6-ylmethoxy]piperidin-2-yl}-1,1-dimethylethyl dimethylcarbamate

Analogously to Method A, 2-[5-[4-(3-methoxypropyl)-3,4-dihydro-2H-benzo[1,4]oxazin-6-ylmethoxy]-1-(toluene-4-sulphonyl)piperidin-2-yl]-1,1-dimethylethyl dimethylcarbamate is reacted. The title compound is identified based on the Rf value.

The starting material is prepared as follows:

### a) 2-[5-[4-(3-Methoxypropyl)-3,4-dihydro-2H-benzo[1,4]oxazin-6-ylmethoxy]-1-(toluene-4-sulphonyl)piperidin-2-yl]-1,1-dimethylethyl dimethylcarbamate

A solution of 0.51 mmol of 1-[5-[4-(3-methoxypropyl)-3,4-dihydro-2H-benzo[1,4]oxazin-6-ylmethoxy]-1-(toluene-4-sulphonyl)piperidin-2-yl]-2-methylpropan-2-ol (Example 15b) in 3 ml of tetrahydrofuran at room temperature is admixed with 2.05 mmol of potassium hydride (60% dispersion in oil), and the reaction mixture is stirred for 1 hour. 5.13 mmol of dimethylcarbamoyl chloride are added, and the reaction mixture is stirred further at room temperature overnight. After 19 hours, the reaction mixture is cooled to 0° C., quenched cautiously with ethanol, diluted with water and extracted with ethyl acetate (2×). The combined organic phases are washed with brine, dried over sodium sulphate and concentrated by evaporation. The title compound is identified from the residue by means of flash chromatography (SiO₂ 60F) based on the Rf value.

### Example 17

### 3-{(2S,5R)-5-[4-(3-Methoxypropyl)-3,4-dihydro-2H-benzo[1,4]oxazin-6-ylmethoxy]piperidin-2-yl}-2,2-dimethyl-N-(tetrahydropyran-4-yl)propionamide

Analogously to Method A, 0.013 mmol of 3-[(2S,5R)-5-[4-(3-methoxypropyl)-3,4-dihydro-2H-benzo[1,4]oxazin-6-ylmethoxy]-1-(toluene-4-sulphonyl)piperidin-2-yl]-2,2-dimethyl-N-(tetrahydropyran-4-yl)propionamide is reacted. The title compound is obtained as a yellow oil. Rf=0.10 (200:20:1 dichloromethane-methanol-25% conc. ammonia); Rt=3.38 (gradient I).

The starting materials are prepared as follows:

### a) 3-[(2S,5R)-5-[4-(3-Methoxypropyl)-3,4-dihydro-2H-benzo[1,4]oxazin-6-ylmethoxy]-1-(toluene-4-sulphonyl)piperidin-2-yl]-2,2-dimethyl-N-(tetrahydropyran-4-yl)propionamide

A solution of 0.016 mmol of 3-[(2S,5R)-5-[4-(3-methoxypropyl)-3,4-dihydro-2H-benzo[1,4]oxazin-6-ylmethoxy]-1-(toluene-4-sulphonyl)piperidin-2-yl]-2,2-dimethylpropionic acid in 2 ml of dichloromethane at room temperature is admixed with 0.032 mmol of (1-chloro-2-methylpropenyl)dimethylamine, stirred for 1.5 hours and then admixed with 0.080 mmol of tetrahydropyran-4-ylamine. After 1 hour, the reaction mixture is diluted with water and extracted with dichloromethane (3×)—the combined organic phases are washed with brine, dried over sodium sulphate and concentrated by evaporation. The title compound is obtained from the residue by means of flash chromatography (SiO₂ 60F) as a yellow oil. Rf=0.20 (2:1 EtOAc-heptane); Rt=4.74 (gradient I).

### b) 3-[(2S,5R)-5-[4-(3-Methoxypropyl)-3,4-dihydro-2H-benzo[1,4]oxazin-6-ylmethoxy]-1-(toluene-4-sulphonyl)piperidin-2-yl]-2,2-dimethylpropionic acid

A solution of 0.037 mmol of methyl 3-[(2S,5R)-5-[4-(3-methoxypropyl)-3,4-dihydro-2H-benzo[1,4]oxazin-6-ylmethoxy]-1-(toluene-4-sulphonyl)piperidin-2-yl]-2,2-dimethylpropionate in 1.5 ml of methanol and 1.5 ml of tetrahydrofuran at room temperature is admixed with 2 ml of 4N NaOH and then heated to 60° C. After 4 hours, the reaction mixture is cooled to room temperature and partitioned between ethyl acetate and 4N HCl. The aqueous phase is extracted with ethyl acetate (2×)—the combined organic phases are washed with brine, dried over sodium sulphate and concentrated by evaporation. The title compound is obtained from the residue by means of flash chromatography (SiO₂ 60F) as a yellow oil. Rf=0.30 (EtOAc); Rt=4.84 (gradient I).

### c) Methyl 3-[(2S,5R)-5-[4-(3-methoxypropyl)-3,4-dihydro-2H-benzo[1,4]oxazin-6-ylmethoxy]-1-(toluene-4-sulphonyl)piperidin-2-yl]-2,2-dimethylpropionate

A solution of 0.64 mmol of methyl (R,S)-3-[(2S,5R)-5-[4-(3-methoxypropyl)-3,4-dihydro-2H-benzo[1,4]oxazin-6-ylmethoxy]-1-(toluene-4-sulphonyl)piperidin-2-yl]-2-methylpropionate (Example 53c) in 20 ml of tetrahydrofuran at −78° C. is admixed with 0.80 mmol of a lithium diisopropylamide solution (0.5M in tetrahydrofuran) within 5 minutes, stirred for 1.5 hours and then admixed with 2.96 mmol of methyl iodide. After 2 hours, the reaction mixture is quenched with 0.5M HCl and extracted with dichloromethane (2×)—the combined organic phases are washed with water (2×), dried over sodium sulphate and concentrated by evaporation. The title compound is obtained from the residue by means of flash chromatography (SiO₂ 60F) as a brown oil. Rf=0.34 (1:1 EtOAc-heptane), Rt=5.47 (gradient I).

Alternative synthesis for methyl 3-[(2S,5R)-5-[4-(3-methoxypropyl)-3,4-dihydro-2H-benzo[1,4]oxazin-6-ylmethoxy]-1-(toluene-4-sulphonyl)piperidin-2-yl]-2,2-dimethylpropionate (Example 17c):

A solution of 4.52 mmol of methyl isobutyrate in 2.5 ml of tetrahydrofuran at −78° C. is admixed with 4.52 mmol of a lithium diisopropylamide solution (0.5M in cyclohexane) and stirred for 30 minutes. 9.04 mmol of hexamethylphosphoramide (HMPA) and a solution of 1.13 mmol of 6-[(3R,6S)-6-bromomethyl-1-(toluene-4-sulphonyl)piperidin-3-yloxymethyl]-4-(3-methoxypropyl)-3,4-dihydro-2H-benzo[1,4]oxazine in 2.5 ml of tetrahydrofuran are added dropwise. The reaction mixture is stirred further at −78° C. for 30 minutes, at −10° C. for 30 minutes and at 0° C. for 3 hours. The reaction mixture is quenched at −10° C. with 1M HCl until a weakly acidic reaction and then extracted with ethyl acetate (3×). The combined organic phases are washed with brine, dried over sodium sulphate and concentrated by evaporation. The title compound is obtained from the residue by means of flash chromatography (SiO₂ 60F) as a brown oil.

### d) 6-[(3R,6S)-6-Bromomethyl-1-(toluene-4-sulphonyl)piperidin-3-yloxymethyl]-4-(3-methoxypropyl)-3,4-dihydro-2H-benzo[1,4]oxazine

A mixture of 1.75 mmol of (2S,5R)-5-[4-(3-methoxypropyl)-3,4-dihydro-2H-benzo[1,4]oxazin-6-ylmethoxy]-1-(toluene-4-sulphonyl)piperidin-2-ylmethyl methanesulphonate (Example 1 h) and 17.5 mmol of lithium bromide in 6 ml of N,N-dimethylformamide is stirred overnight at 65° C. Further lithium bromide (17.5 mmol) is added after 16 hours. After a total of 19 hours, the reaction mixture is cooled to room temperature, diluted with water and extracted with ethyl acetate (3×). The combined organic phases are dried over sodium sulphate and concentrated by evaporation. The title compound is obtained from the residue by means of flash chromatography (SiO₂ 60F) as a yellow oil. Rf=0.45 (1:1 EtOAc-heptane); Rt=5.42 (gradient I).

Alternative synthesis for 3-[(2S,5R)-5-[4-(3-methoxypropyl)-3,4-dihydro-2H-benzo[1,4]oxazin-6-ylmethoxy]-1-(toluene-4-sulphonyl)piperidin-2-yl]-2,2-dimethylpropionic acid methyl ester (Example 17c)

Analogously to Method H, 6.77 mmol of 3-[(2S,5R)-5-hydroxy-1-(toluene-4-sulphonyl)piperidin-2-yl]-2,2-dimethylpropionic acid methyl ester and 7.10 mmol of 6-chloromethyl-4-(3-methoxypropyl)-4H-benzo[1,4]oxazin-3-one [857272-02-7] are reacted. The title compound is obtained as a yellow oil. Rf=0.40 (2:1 EtOAc-heptane); Rt=5.14 (gradient I).

### e) 3-[(2S,5R)-5-Hydroxy-1-(toluene-4-sulphonyl)piperidin-2-yl]-2,2-dimethylpropionic acid methyl ester

A solution of 11.03 mmol of 3-[(2S,5R)-5-(4-methoxybenzyloxy)-1-(toluene-4-sulphonyl)piperidin-2-yl]-2,2-dimethylpropionic acid methyl ester is dissolved in 100 ml of methanol. 1.03 mmol of Pd/C (10%) are added under argon. The reaction system is purged 3 times with hydrogen, and the reaction mixture is stirred under an atmosphere of hydrogen for 16 hours, then filtered through Hyflo, washing with methanol. The filtrate is concentrated under reduced pressure and purified by flash chromatography (SiO₂ 60F), to afford the title compound as a colourless oil. Rf=0.38 (EtOAc-heptane 2:1); Rt=4.07 (gradient I).

### f) 3-[(2S,5R)-5-(4-Methoxybenzyloxy)-1-(toluene-4-sulphonyl)piperidin-2-yl]-2,2-dimethylpropionic acid methyl ester

A solution of 44.20 mmol of potassium tert-butoxide and 2.21 mmol of 18-crown-6 is dissolved in 200 ml of dry tetrahydrofuran and cooled to −78° C. A solution of 11.05 mmol of 3-[(2S,5R)-5-(4-methoxybenzyloxy)-1-(toluene-4-sulphonyl)piperidin-2-yl]-propionic acid methyl ester in 50 ml of dry tetrahydrofuran is added dropwise. The reaction mixture is stirred at −78° C. for 1 hour, 44.20 mmol of iodomethane are then added and the reaction mixture is stirred for another 1.5 hours, then quenched with 0.2N HCl. The reaction mixture is allowed to reach room temperature, diluted with 0.2N HCl, extracted with dichloromethane (3×). The combined organic extracts are dried over sodium sulphate and concentrated to afford the crude product as a colourless oil. Rf=0.60 (EtOAc-heptane 2:1); Rt=5.43 (gradient I).

### g) 3-[(2S,5R)-5-(4-Methoxybenzyloxy)-1-(toluene-4-sulphonyl)piperidin-2-yl]-propionic acid methyl ester

28.48 mmol of sodium borohydride are slowly added to a solution of 14.24 mmol of (E)-3-[(2S,5R)-5-(4-methoxybenzyloxy)-1-(toluene-4-sulphonyl)piperidin-2-yl]acrylic acid methyl ester and 1.424 mmol of nickel chloride hexahydrate in 500 ml of methanol, at 0° C. The reaction mixture is stirred for 1 hour, then filtered through Hyflo, washing with methanol. The filtrate is concentrated under reduced pressure, and purified by flash chromatography (SiO₂ 60F) to afford the title compound as a colourless oil. Rf=0.36 (EtOAc-heptane); Rt=5.03 (gradient I).

### h) (E)-3-[(2S,5R)-5-(4-Methoxybenzyloxy)-1-(toluene-4-sulphonyl)piperidin-2-yl]-acrylic acid methyl ester

A suspension of 21.07 mmol of sodium hydride (60% in oil) in 200 ml of tetrahydrofuran is cooled to 0° C. and treated with a solution of 22.47 mmol of trimethyl phosphonoacetate in 40 ml of tetrahydrofuran. The reaction mixture is stirred at 0° C. for 30 minutes, before the addition of 14.044 mmol of (2S,5R)-5-(4-methoxybenzyloxy)-1-(toluene-4-sulphonyl)piperidine-2-carbaldehyde in 40 ml of tetrahydrofuran. After 1 hour, the reaction mixture is quenched with 0.5N HCl, extracted with dichloromethane (3×), dried over sodium sulphate and concentrated under reduced pressure to afford the crude product as a yellow oil. Rf=0.57 (EtOAc-heptane 2:1); Rt=5.01 (gradient I).

### i) (2S,5R)-5-(4-Methoxybenzyloxy)-1-(toluene-4-sulphonyl)piperidine-2-carbaldehyde

A solution of 20.844 mmol of oxalyl chloride in 100 ml of dichloromethane at −78° C. is treated with 41.69 mmol of N,N-dimethyl sulphoxide. The reaction mixture is stirred at −78° C. for 45 minutes, a solution of 13.90 mmol of [(2S,5R)-5-(4-methoxybenzyloxy)-1-(toluene-4-sulphonyl)piperidin-2-yl]methanol in 30 ml of dichloromethane is then added dropwise, and the reaction mixture is stirred for another hour at −78° C. 97.27 mmol of triethylamine are then added, the reaction mixture is allowed to reach room temperature over 30 minutes, poured onto 0.5N HCl, extracted with dichloromethane (3×), dried over sodium sulphate and concentrated under reduced pressure to afford the title compound as a beige sticky solid. Rf=0.57 (EtOAc-heptane 2:1); Rt=4.78 (gradient I).

### h) [(2S,5R)-5-(4-Methoxybenzyloxy)-1-(toluene-4-sulphonyl)piperidin-2-yl]methanol

Following Method G, 21.123 mmol of (2S,5R)-5-(4-methoxy-benzyloxy)-1-(toluene-4-sulphonyl)-2-triisopropylsilanyloxymethylpiperidine are reacted with 25.35 mmol of tetrabutylammonium fluoride to afford the title compound as a yellow oil. Rf=0.13 (EtOAc-heptane 1:1); Rt=4.34 (gradient I).

### i) (2S,5R)-5-(4-Methoxybenzyloxy)-1-(toluene-4-sulphonyl)-2-triisopropylsilanyloxymethylpiperidine

Following Method H, 25.13 mmol of (3R,6S)-1-(toluene-4-sulphonyl)-6-triisopropylsilanyloxymethylpiperidin-3-ol (Example 11) are reacted with 27.643 mmol of 4-methoxybenzyl chloride to afford the title compound as an orange oil. Rf=0.60 (EtOAc-heptane 1:1); Rt=7.01 (gradient I).

The following compounds are prepared in an analogous manner by the process described in Example 17:
18 3-{(2S,5R)-5-[4-(3-Methoxypropyl)-3,4-dihydro-2H-benzo[1,4]oxazin-6-ylmethoxy]piperidin-2-yl}-2,2-dimethyl-1-morpholin-4-ylpropan-1-one
19 3-{(2S,5R)-5-[4-(3-Methoxypropyl)-3,4-dihydro-2H-benzo[1,4]oxazin-6-ylmethoxy]piperidin-2-yl}-2,2-dimethyl-N-(tetrahydropyran-4-ylmethyl)-propionamide
20 3-{(2S,5R)-5-[4-(3-Methoxypropyl)-3,4-dihydro-2H-benzo[1,4]oxazin-6-ylmethoxy]piperidin-2-yl}-2,2-dimethyl-N—(S)-1-piperidin-3-ylmethylpropionamide
21 3-{(2S,5R)-5-[4-(3-Methoxypropyl)-3,4-dihydro-2H-benzo[1,4]oxazin-6-ylmethoxy]piperidin-2-yl}-2,2-dimethyl-N—(R)-1-piperidin-3-ylmethylpropionamide
22 3-{(2S,5R)-5-[4-(3-Methoxypropyl)-3,4-dihydro-2H-benzo[1,4]oxazin-6-ylmethoxy]piperidin-2-yl}-2,2-dimethyl-N—(R)-1-piperidin-2-ylmethylpropionamide
23 3-{(2S,5R)-5-[4-(3-Methoxypropyl)-3,4-dihydro-2H-benzo[1,4]oxazin-6-ylmethoxy]piperidin-2-yl}-2,2-dimethyl-N—(S)-1-piperidin-2-ylmethylpropionamide
24 3-{(2S,5R)-5-[4-(3-Methoxypropyl)-3,4-dihydro-2H-benzo[114]oxazin-6-ylmethoxy]piperidin-2-yl}-22, N-trimethylpropionamide
25 N-Cyclopropylmethyl-3-{(2S,5R)-5-[4-(3-methoxypropyl)-3,4-dihydro-2H-benzo[1,4]oxazin-6-ylmethoxy]piperidin-2-yl}-2,2-dimethylpropionamide
26 N-(2-Carbamoyl-2-methylpropyl)-3-{(2S,5R)-5-[4-(3-methoxypropyl)-3,4-dihydro-2H-benzo[1,4]oxazin-6-ylmethoxy]piperidin-2-yl}-2,2-dimethylpropionamide
27 3-{(2S,5R)-5-[4-(3-Methoxypropyl)-3,4-dihydro-2H-benzo[1,4]oxazin-6-ylmethoxy]piperidin-2-yl}-2,2-dimethyl-N-[(1S,5R,6S)-1-(3-oxabicyclo[3.1.0]hex-6-yl)methyl]propionamide
28 3,4-dihydro-2H-Benzo[1,4]oxazin-6-ylmethoxy]piperidin-2-yl}-2,2-dimethyl-N-(1R,5S,6R)-3-oxa-bicyclo[3.1.0]hex-6-ylpropionamide
29 3-{(2S,5R)-5-[4-(3-Methoxypropyl)-3,4-dihydro-2H-benzo[1,4]oxazin-6-ylmethoxy]piperidin-2-yl}-2,2-dimethyl-N—[(S)-1-(tetrahydrofuran-2-yl)methyl]-propionamide
30 N-(4-Methoxycyclohexyl)-3-{(2S,5R)-5-[4-(3-methoxypropyl)-3,4-dihydro-2H-benzo[1,4]oxazin-6-ylmethoxy]piperidin-2-yl}-2,2-dimethylpropionamide
31 N—((R-2-Hydroxy-3-methoxypropyl)-3-{(2S,5R)-5-[4-(3-methoxypropyl)-3,4-dihydro-2H-benzo[1,4]oxazin-6-ylmethoxy]piperidin-2-yl}-2,2-dimethylpropionamide
32 3-{(2S,5R)-5-[4-(3-Methoxypropyl)-3,4-dihydro-2H-benzo[1,4]oxazin-6-ylmethoxy]piperidin-2-yl}-2,2-dimethyl-N-(2-morpholin-4-ylethyl)-propionamide
33 3-{(2S,5R)-5-[4-(3-Methoxypropyl)-3,4-dihydro-2H-benzo[1,4]oxazin-6-ylmethoxy]piperidin-2-yl}-2,2-dimethyl-N—[(S)-1-(6-oxaspiro[2,5]oct-1-yl)methyl]propionamide
34 3-{(2S,5R)-5-[4-(3-Methoxypropyl)-3,4-dihydro-2H-benzo[1,4]oxazin-6-ylmethoxy]piperidin-2-yl}-2,2-dimethyl-N—[(R)-1-(6-oxaspiro[2.5]oct-1-yl)methyl]propionamide
94 3-{(2S,5R)-5-[spiro[(4-(3-Methoxypropyl)-3-oxo-3,4-dihydro-2H-benzo[1,4]oxazine)-2,4′-(tetrahydropyran)]-6-ylmethoxy]piperidin-2-yl}-2,2-dimethyl-N-(tetrahydropyran-4-ylmethyl)propionamide and
95 3-{(2S,5R)-5-[spiro[(4-(3-Methoxypropyl)-3,4-dihydro-2H-benzo[1,4]oxazine)-2,4′-(tetrahydropyran)]-6-ylmethoxy]piperidin-2-yl}-2,2-dimethyl-N-(tetrahydropyran-4-ylmethyl)propionamide

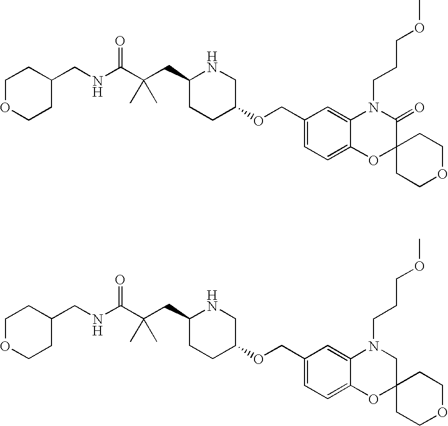

Starting from 6-bromomethylspiro[4-(3-methoxypropyl)-4H-benzo[1,4]oxazin-2,4′-(tetrahydropyran)]-3-one.

The starting materials are prepared as follows:

### a) 6-Bromomethylspiro[4-(3-methoxypropyl)-4H-benzo[1,4]oxazin-2,4′-(tetrahydropyran)]-3-one

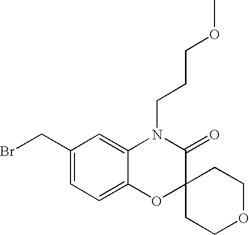

A solution of 2.086 mmol of spiro[4-(3-methoxypropyl)-6-methyl-4H-benzo[1,4]oxazine]-2,4′-(tetrahydropyran)]-3-one in 20 ml of carbon tetrachloride is heated at 70° C. and treated with 3.338 mmol of N-bromosuccinimide, 0.073 mmol of 2,2′-azobis(2-methylpropionitrile) and 0.052 mmol of benzoyl peroxide. The reaction mixture is stirred at 80° C. for 60 hours, then cooled to 0° C., filtered through Hyflo, and concentrated under reduced pressure. Purification by flash chromatography (SiO₂ 60F) affords the title compound as a colourless oil. Rf=0.33 (EtOAc-heptane 1:1); Rt=4.38 (gradient I).

### b) Spiro[4-(3-Methoxypropyl)-6-methyl-4H-benzo[1,4]oxazine]-2,4′-(tetrahydropyran)]-3-one

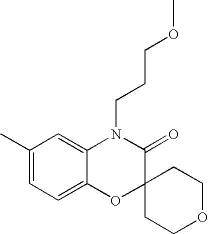

A solution of 6.72 mmol of 6-methylspiro[4H-benzo[1,4]oxazin-2,4′-(tetrahydropyran)]-3-one in 80 ml of acetonitrile is treated with 13.44 mmol of 1-chloro-3-methoxypropane [36215-07-3], 37.03 mmol of potassium fluoride on alox and 0.134 mmol of potassium iodide. The reaction mixture is stirred for 18 hours, cooled to room temperature and filtered through Hyflo. Purification by flash chromatography (SiO₂ 60F) affords the title compound as a colourless oil. Rf=0.47 (EtOAc-heptane 1:1); Rt=4.29 (gradient I).

### c) 6-Methylspiro[4H-benzo[1,4]oxazine-2,4′-(tetrahydropyran)]-3-one

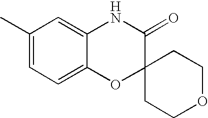

A solution of 19.39 mmol of 4-(4-methyl-2-nitrophenoxy)tetrahydropyran-4-carboxylic acid in 60 ml of acetic acid and 6 ml of water at 50° C. is treated with 110.55 mmol of iron powder. The reaction mixture is stirred at 50° C. for 18 hours, cooled to room temperature and filtered through Hyflo. The filter cake is washed with dichloromethane and water. The phases are separated, the aqueous phase is re-extracted 3× with dichloromethane. The combined organic extracts are dried over sodium sulphate and concentrated under reduced pressure. Purification by flash chromatography (SiO₂ 60F) affords the title compound as a white solid. Rf=0.38 (EtOAc-heptane 1:1); Rt=3.52 (gradient I).

### d) 4-(4-Methyl-2-nitrophenoxy)tetrahydropyran-4-carboxylic acid

To a solution of 19.39 mmol of 4-methyl-2-nitrophenol in 70 ml of tetrahydrofuran is added 165 mmol of powdered NaOH and the reaction mixture is stirred at room temperature for 15 minutes. 175 mmol of tetrahydro-4H-pyran-4-one are added, and the mixture is cooled to 0° C. 78 mmol of chloroform are added dropwise, and the reaction mixture is stirred at 0° C. for 1 hour, then at room temperature for 18 hours. The reaction mixture is partitioned between water and dichloromethane. The aqueous phase is re-extracted with dichloromethane (3×). The aqueous phase is acidified with 4M HCl, then extracted 3× with dichloromethane. These organic extracts are dried over sodium sulphate, and concentrated to afford the crude title compound as a brown oil. Rf=0.15 (EtOAc-heptane 1:1).
96 3-{(2S,5R)-5-[4-(3-Methoxypropyl)-2-spirocyclopropyl-3-oxo-3,4-dihydro-2H-benzo[1,4]oxazin-6-ylmethoxy]piperidin-2-yl}-2,2-dimethyl-N-(tetrahydropyran-4-ylmethyl)propionamide and
97 3-{(2S,5R)-5-[4-(3-Methoxypropyl)-2-spirocyclopropyl-3,4-dihydro-2H-benzo[1,4]oxazin-6-ylmethoxy]piperidin-2-yl}-2,2-dimethyl-N-(tetrahydropyran-4-ylmethyl)propionamide

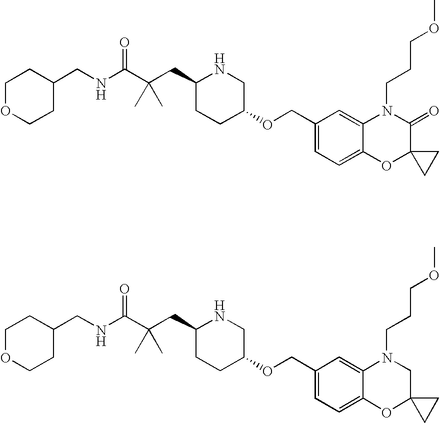

Starting from 6-hydroxymethyl-4-(3-methoxypropyl)-2-spirocyclopropyl-4H-benzo[1,4]oxazin-3-one.

The starting materials are prepared as follows:

### a) 6-Hydroxymethyl-4-(3-methoxypropyl)-2-spirocyclopropyl-4H-benzo[1,4]oxazin-3-one

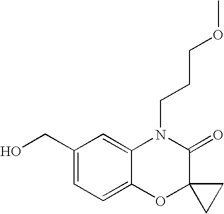

A solution of 4.236 mmol of 4-(3-methoxypropyl-2-spirocyclopropyl-3-oxo-3,4-dihydro-2H-benzo[1,4]oxazine-6-carboxylic acid in 10 ml of tetrahydrofuran is treated with 4.871 mmol of triethylamine and cooled to 0° C. 4.66 mmol of ethyl chloroformate is added dropwise and the reaction mixture is stirred for 1.5 hours at 0° C. The mixture is then diluted with dichloromethane, and washed with cold 0.5N HCl. The aqueous layer is re-extracted with dichloromethane (2×), the combined organic layers are dried over sodium sulphate, and concentrated under reduced pressure. This intermediate is re-dissolved in tetrahydrofuran (10 ml, including washings), and added dropwise to a solution of 10.6 mmol of sodium borohydride in 4 ml of water at 0° C. Once the addition is completed, the reaction mixture is warmed to room temperature, and stirred for 2 hours. The reaction mixture is cautiously acidified with 1N HCl, and extracted with tert-butyl methyl ether. The organic extracts are washed with 1N NaOH and brine, dried over sodium sulphate and concentrated. This affords the title compound as a colourless oil. Rf=0.15 (EtOAc-heptane 2:1); Rt=3.22 (gradient I).

### b) 4-(3-Methoxypropyl)-2-spirocyclopropyl-3-oxo-3,4-dihydro-2H-benzo[1,4]oxazine-6-carboxylic acid

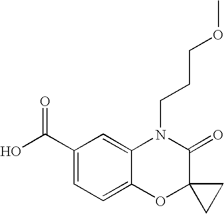

Following the procedure for Example 17b, 4.389 mmol of 4-(3-methoxypropyl)-2-spirocyclopropyl-3-oxo-3,4-dihydro-2H-benzo[1,4]oxazine-6-carboxylic acid methyl ester are reacted. The title compound is isolated as a white solid. Rf=0.10 (EtOAc-heptane 1:1); Rt=3.48 (gradient I).

### c) 4-(3-Methoxypropyl)-2-spirocyclopropyl-3-oxo-3,4-dihydro-2H-benzo[1,4]oxazine-6-carboxylic acid methyl ester

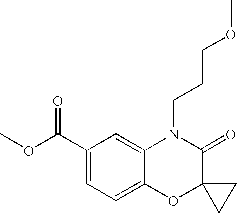

Following the procedure for Example 94b, 10.76 mmol of 2-spirocyclopropyl-3-oxo-3,4-dihydro-2H-benzo[1,4]oxazine-6-carboxylic acid methyl ester are reacted to afford the title compound as a colourless oil. Rf=0.45 (EtOAc-heptane 1:1); Rt=4.30 (gradient I).

### d) 2-Spirocyclopropyl-3-oxo-3,4-dihydro-2H-benzo[1,4]oxazine-6-carboxylic acid methyl ester

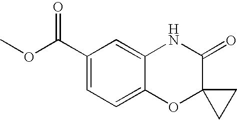

Following the procedure for Example 94c, 11.22 mmol of 4-(1-methoxycarbonylcyclopropoxy)-3-nitrobenzoic acid methyl ester are reacted to afford the title compound as a pale yellow solid. Rf=0.45 (EtOAc-heptane 1:1); Rt=3.56 (gradient I).

### e) 4-(1-Methoxycarbonylcyclopropoxy)-3-nitrobenzoic acid methyl ester

To a suspension of 20.81 mmol of sodium hydride in 10 ml of dry N,N-dimethylformamide at 0° C. is added a solution of 17.34 mmol of methyl 1-hydroxy-1-cyclopropane carboxylate in 10 ml of dry N,N-dimethylformamide. The reaction mixture is stirred at 0° C. for 1 hour, before the addition of 27.74 mmol of methyl 4-fluoro-3-nitrobenzoate. The reaction mixture is stirred at 0° C. for 1 hour, then at room temperature for 3 hours, poured onto saturated aqueous ammonium chloride, extracted with tert-butyl methyl ether (3×), dried over sodium sulphate and concentrated. Purification by flash chromatography (SiO₂ 60F) affords the title compound as a thick yellow oil. Rf=0.48 (EtOAc-heptane 1:1); Rt=4.18 (gradient I).
98 3-{(2S,5R)-5-[9-(3-Methoxypropyl)-9H-carbazol-2-ylmethoxy]piperidin-2-yl}-2,2-dimethyl-N-(tetrahydropyran-4-ylmethyl)propionamide

Starting from [9-(3-methoxypropyl)-9H-carbazol-2-yl]methanol.

The starting material is prepared as follows:

### a) [9-(3-Methoxypropyl)-9H-carbazol-2-yl]methanol

A solution of 8.307 mmol of 9-(3-methoxypropyl)-9H-carbazole-2-carboxylic acid methyl ester [925930-95-6] in 65 ml of tetrahydrofuran is cooled to 0° C. and treated over 1 hour with 83.07 mmol of lithium aluminium hydride. The reaction is then stirred at room temperature for 2 hours, quenched cautiously with 6 ml of water, followed by 2 ml of 2N NaOH and 20 ml of water. The resulting solution is stirred at room temperature for 18 hours, filtered through Hyflo, and concentrated under reduced pressure. Purification by flash chromatography (SiO₂ 60F) affords the title compound as a yellow oil. Rf=0.43 (EtOAc); Rt=4.18 (gradient I).

### Example 35

### 3-{(2S,5R)-5-[2-(4-Methoxybutyl)-6-methylpyridin-4-ylmethoxy]piperidin-2-yl}-2,2-dimethyl-N-(tetrahydropyran-4-ylmethyl)propionamide

Analogously to Method A, 3-[(2S,5R)-5-[2-(4-methoxybutyl)-6-methylpyridin-4-ylmethoxy]-1-(toluene-4-sulphonyl)piperidin-2-yl]-2,2-dimethyl-N-(tetrahydropyran-4-ylmethyl)propionamide is reacted. The title compound is identified based on the Rf value.

The starting materials are prepared as follows:

### a) 3-[(2S,5R)-5-[2-(4-Methoxybutyl)-6-methylpyridin-4-ylmethoxy]-1-(toluene-4-sulphonyl)piperidin-2-yl]-2,2-dimethyl-N-(tetrahydropyran-4-ylmethyl)propionamide

Analogously to Example 23a, 3-[(2S,5R)-5-[2-(4-methoxybutyl)-6-methylpyridin-4-ylmethoxy]-1-(toluene-4-sulphonyl)piperidin-2-yl]-2,2-dimethylpropionic acid and C-(tetrahydropyran-4-yl)methylamine are reacted. The title compound is identified based on the Rf value.

### b) 3-[(2S,5R)-5-[2-(4-Methoxybutyl)-6-methylpyridin-4-ylmethoxy]-1-(toluene-4-sulphonyl)piperidin-2-yl]-2,2-dimethylpropionic acid

Analogously to Example 17b, methyl 3-[(2S,5R)-5-[2-(4-methoxybutyl)-6-methylpyridin-4-ylmethoxy]-1-(toluene-4-sulphonyl)piperidin-2-yl]-2,2-dimethylpropionate is reacted. The title compound is identified based on the Rf value.

### c) Methyl 3-[(2S,5R)-5-[2-(4-methoxybutyl)-6-methylpyridin-4-ylmethoxy]-1-(toluene-4-sulphonyl)piperidin-2-yl]-2,2-dimethylpropionate

Analogously to Example 17c (alternative synthesis), 4-[(3R,6S)-6-bromomethyl-1-(toluene-4-sulphonyl)piperidin-3-yloxymethyl]-2-(4-methoxybutyl)-6-methylpyridine and methyl isobutyrate are reacted. The title compound is identified based on the Rf value.

### d) 4-[(3R,6S)-6-Bromomethyl-1-(toluene-4-sulphonyl)piperidin-3-yloxymethyl]-2-(4-methoxybutyl)-6-methylpyridine

Analogously to Example 17d, (2S,5R)-5-[2-(4-methoxybutyl)-6-methylpyridin-4-ylmethoxy]-1-(toluene-4-sulphonyl)piperidin-2-ylmethyl methanesulphonate is reacted. The title compound is identified based on the Rf value.

### e) (2S,5R)-5-[2-(4-Methoxybutyl)-6-methylpyridin-4-ylmethoxy]-1-(toluene-4-sulphonyl)piperidin-2-ylmethyl methanesulphonate

Analogously to Method E, [(2S,5R)-5-[2-(4-methoxybutyl)-6-methylpyridin-4-ylmethoxy]-1-(toluene-4-sulphonyl)piperidin-2-yl]methanol is reacted. The title compound is identified based on the Rf value.

### f) [(2S,5R)-5-[2-(4-Methoxybutyl)-6-methylpyridin-4-ylmethoxy]-1-(toluene-4-sulphonyl)piperidin-2-yl]methanol

Analogously to Method G, 2-(4-methoxybutyl)-6-methyl-4-[(3R,6S)-1-(toluene-4-sulphonyl)-6-triisopropylsilanyloxymethylpiperidin-3-yloxymethyl]pyridine is reacted. The title compound is identified based on the Rf value.

### g) 2-(4-Methoxybutyl)-6-methyl-4-[(3R,6S)-1-(toluene-4-sulphonyl)-6-triisopropylsilanyloxymethylpiperidin-3-yloxymethyl]pyridine

Analogously to Method H, (3R,6S)-1-(toluene-4-sulphonyl)-6-triisopropylsilanyloxymethylpiperidin-3-ol (Example 11) and 4-bromomethyl-2-(4-methoxybutyl)-6-methylpyridine are reacted. The title compound is identified based on the Rf value.

### h) 4-Bromomethyl-2-(4-methoxybutyl)-6-methylpyridine

Analogously to Method K, [2-(4-methoxybutyl)-6-methylpyridin-4-yl]methanol is reacted. The title compound is identified based on the Rf value.

### i) [2-(4-Methoxybutyl)-6-methylpyridin-4-yl]methanol

Analogously to Method S, 2-(4-methoxybutyl)-6-methylpyridine-4-carbaldehyde is reacted. The title compound is identified based on the Rf value.

### j) 2-(4-Methoxybutyl)-6-methylpyridine-4-carbaldehyde

Analogously to Method W, 4-bromo-2-(4-methoxybutyl)-6-methylpyridine is reacted.

The title compound is identified based on the Rf value.

### k) 4-Bromo-2-(4-methoxybutyl)-6-methylpyridine

A solution of 0.208 mmol of 4-bromo-2-(4-methoxybut-1-ynyl)-6-methylpyridine in 1 ml of tetrahydrofuran is admixed with 0.208 mmol of triethylamine and 0.008 mmol of platinum oxide hydrate and hydrogenated under a hydrogen atmosphere at standard pressure for 4 hours. The mixture is filtered from the catalyst; the filtercake is washed with tetrahydrofuran. The clear solution is washed with brine, dried over sodium sulphate and concentrated by evaporation. The title compound is identified from the residue by means of flash chromatography (SiO₂ 60F) based on the Rf value.

### l) 4-Bromo-2-(4-methoxybut-1-ynyl)-6-methylpyridine

1.2 mmol of diisopropylamine and 1.1 mmol of 4-methoxybutyne [158878-83-2] are added to a mixture of 1 mmol of 2,4-dibromo-6-methylpyridine [79055-52-0], 0.07 mmol of copper(1) iodide and 0.07 mmol of bis(triphenylphosphine)palladium chloride in 3.6 ml of dioxane. The mixture is stirred at room temperature for 1.5 hours and diluted with water. The mixture is extracted with tert-butyl methyl ether (3×)—the combined organic phases are washed with brine, dried over sodium sulphate and concentrated by evaporation. The title compound is identified from the residue by means of flash chromatography (SiO₂ 60F) based on the Rf value.

The following compounds are prepared in an analogous manner by the process described in Example 35:
36 3-{(2S,5R)-5-[3-(4-Methoxybutyl)-5-methylbenzyloxy]piperidin-2-yl}-2,2-dimethyl-N-(tetrahydropyran-4-ylmethyl)propionamide

Starting-from-1-bromo-3-(4-methoxybutyl)-5-methylbenzene.

The starting materials are prepared as follows:

### a) 1-Bromo-3-(4-methoxybutyl)-5-methylbenzene

A solution of 0.208 mmol of 1-bromo-3-((E,Z)-4-methoxybut-1-enyl)-5-methylbenzene in 1 ml of ethanol is admixed with 0.008 mmol of palladium on carbon (10%) and hydrogenated under a hydrogen atmosphere at standard pressure for 2 hours. The mixture is filtered from the catalyst, and the filtercake is washed with ethanol and concentrated by evaporation. The title compound is identified from the residue by means of flash chromatography (SiO₂ 60F) based on the Rf value.

### b) 1-Bromo-3-((E,Z)-4-methoxybut-1-enyl)-5-methylbenzene

3.45 mmol of sodium bis(trimethylsilyl)amide are added at 0° C. to a suspension of 3.45 mmol of (3-methoxypropyl)triphenylphosphonium bromide [111088-69-8] in 7.5 ml of tetrahydrofuran. The solution is stirred at 0° C. for 30 minutes and 2.3 mmol of 3-bromo-5-methylbenzaldehyde [1611-92-3] are added. The reaction mixture is warmed to room temperature over 30 minutes and diluted with tert-butyl methyl ether. The mixture is washed with 1M sodium hydrogencarbonate solution (2×), dried over sodium sulphate and concentrated by evaporation. The title compound is identified from the residue by means of flash chromatography (SiO₂ 60F) based on the Rf value.
37 3-{(2S,5R)-5-[4-Methoxy-3-(3-methoxypropoxy)benzyloxy]piperidin-2-yl}-2,2-dimethyl-N-(tetrahydropyran-4-ylmethyl)propionamide

Starting from 4-bromomethyl-1-methoxy-2-(3-methoxypropoxy)benzene [172900-73-1].
38 3-{(2S,5R)-5-[4-(3-Methoxypropyl)-2,2-dimethyl-2H-chromen-6-ylmethoxy]-piperidin-2-yl}-2,2-dimethyl-N-(tetrahydropyran-4-ylmethyl)propionamide

Starting from 6-chloromethyl-4-(3-methoxypropyl-2,2-dimethyl-2H-chromene.

The starting materials are prepared as follows:

### a) 6-Chloromethyl-4-(3-methoxypropyl)-2,2-dimethyl-2H-chromene

1.720 ml of 1-chloro-N,N-2-trimethylpropenylamine are added dropwise at room temperature to a solution of 0.225 g of [4-(3-methoxypropyl)-2,2-dimethyl-2H-1-chromen-6-yl]methanol in 5 ml of dichloromethane. The reaction solution is stirred at room temperature for 60 minutes and then concentrated by evaporation. The title compound is obtained from the residue by means of flash chromatography (SiO₂ 60F) and used immediately in the next stage.

### b) [4-(3-Methoxypropyl)-2,2-dimethyl-2H-chromen-6-yl]methanol

A solution of 5.200 g of 4-(3-methoxypropyl)-2,2-dimethyl-2H-chromene-6-carbaldehyde in 60 ml of dry tetrahydrofuran is admixed at 0° C. with 0.294 g of lithium borohydride in portions. The reaction mixture is stirred at 0° C. for 1 hour and then admixed with 5 ml of methanol and 50 ml of water, and extracted with tert-butyl methyl ether (3×). The combined organic phases are washed with brine, dried over sodium sulphate and concentrated by evaporation. The title compound is obtained from the residue by means of flash chromatography (SiO₂ 60F) as a colourless oil. Rf=0.28 (1:1 EtOAc-heptane); Rt=4.79 (gradient I).

### c) 4-(3-Methoxypropyl)-2,2-dimethyl-2H-chromene-6-carbaldehyde

A solution of 7.500 g of 6-[1,3]dioxolan-2-yl-4-(3-methoxypropyl)-2,2-dimethylchroman-4-ol in 100 ml of methanol is admixed with 0.560 g of p-toluenesulphonic acid monohydrate. The reaction solution is heated to reflux over 1 hour and then stirred at room temperature over 16 hours. The reaction solution is concentrated by evaporation and the title compound is obtained as a colourless oil from the residue by means of flash chromatography (SiO₂ 60F). Rf=0.28 (1:2 EtOAc-heptane); Rt=4.73 (gradient I).

### d) 6-[1,3]Dioxolan-2-yl-4-(3-methoxypropyl)-2,2-dimethylchroman-4-ol

A solution of 5.700 g of 6-[1,3]dioxolan-2-yl-2,2-dimethylchroman-4-one [221301-35-5] in 60 ml of tetrahydrofuran is admixed with 36 ml of 3-methoxypropylmagnesium chloride solution [14202-12-1] (approx. 1.84M in tetrahydrofuran). The reaction mixture is stirred at room temperature for 30 minutes, quenched with saturated aqueous sodium carbonate solution and extracted with tert-butyl methyl ether (3×). The combined organic phases are washed with brine, dried over sodium sulphate and concentrated by evaporation. The crude product is obtained as a yellowish oil and used in the next stage without further purification. Rt=5.66 (gradient I).
39 3-{(2S,5R)-5-[4-(3-Methoxypropyl)-2,2-dimethyl-3-oxo-3,4-dihydro-2H-benzo[1,4]oxazin-6-ylmethoxy]piperidin-2-yl}-2,2-dimethyl-N-(tetrahydropyran-4-ylmethyl)propionamide

Starting from 6-hydroxymethyl-4-(3-methoxypropyl)-2,2-dimethyl-4H-benzo[1,4]oxazin-3-one [857281-71-1].
40 3-{(2S,5R)-5-[4-(3-Methoxypropyl)-2,2-dimethyl-3,4-dihydro-2H-benzo[1,4]oxazin-6-ylmethoxy]piperidin-2-yl}-2,2-dimethyl-N-(tetrahydropyran-4-ylmethyl)propionamide

Starting from [4-(3-methoxypropyl)-2,2-dimethyl-3,4-dihydro-2H-benzo[1,4]oxazin-6-yl]methanol.

The starting material is prepared as follows:

### a) [4-(3-Methoxypropyl)-2,2-dimethyl-3,4-dihydro-2H-benzo[1,4]oxazin-6-yl]methanol

Analogously to Method F, 6-hydroxymethyl-4-(3-methoxypropyl)-2,2-dimethyl-4H-benzo[1,4]oxazin-3-one [857281-72-2] is reacted. The title compound is identified based on the Rf value.
41 3-{(2S,5R)-5-[4-(3-Methoxypropyl)-2,2-dimethyl-3-oxo-3,4-dihydro-2H-benzo[1,4]oxazin-6-ylmethoxy]piperidin-2-yl}-2,2,N-trimethylpropionamide

Starting from 6-hydroxymethyl-4-(3-methoxypropyl)-2,2-dimethyl-4H-benzo[1,4]oxazin-3-one [857281-71-1].
42 3-{(2S,5R)-5-[4-Fluoro-3-(3-methoxypropoxy)benzyloxy]piperidin-2-yl}-2,2-dimethyl-N-(tetrahydropyran-4-ylmethyl)propionamide

Starting from 4-chloromethyl-1-fluoro-2-(3-methoxypropoxy)benzene [857272-79-8].
43 3-{(2S,5R)-5-[3-(3-Methoxypropoxy)-4-methylbenzyloxy]piperidin-2-yl}-2,2-dimethyl-N-(tetrahydropyran-4-ylmethyl)propionamide

Starting from 4-chloromethyl-2-(3-methoxypropoxy)-1-methylbenzene [85727246-9].

### Example 44

### 3-{(2S,5R)-5-[4-(3-Methoxypropyl)-2-methyl-2-phenyl-3,4-dihydro-2H-benzo[1,4]oxazin-6-ylmethoxy]piperidin-2-yl}-2,2-dimethyl-N-(tetrahydropyran-4-ylmethyl)propionamide

Analogously to Method A, 3-[(2S,5R)-5-[4-(3-methoxypropyl)-2-methyl-2-phenyl-3,4-dihydro-2H-benzo[1,4]oxazin-6-ylmethoxy]-1-(toluene-4-sulphonyl)piperidin-2-yl]-2,2-dimethyl-N-(tetrahydropyran-4-ylmethyl)propionamide is reacted. The title compound is identified based on the Rf value.

The starting materials are prepared as follows:

### a) 3-[(2S,5R)-5-[4-(3-Methoxypropyl)-2-methyl-2-phenyl-3,4-dihydro-2H-benzo[1,4]oxazin-6-ylmethoxy]-1-(toluene-4-sulphonyl)piperidin-2-yl]-2,2-dimethyl-N-(tetrahydropyran-4-ylmethyl)propionamide

Analogously to Example 17a, 3-[(2S,5R)-5-[4-(3-methoxypropyl)-2-methyl-2-phenyl-3,4-dihydro-2H-benzo[1,4]oxazin-6-ylmethoxy]-1-(toluene-4-sulphonyl)piperidin-2-yl]-2,2-dimethylpropionic acid and C-(tetrahydropyran-4-yl)methylamine are reacted. The title compound is identified based on the Rf value.

### b) 3-[(2S,5R)-5-[4-(3-Methoxypropyl)-2-methyl-2-phenyl-3,4-dihydro-2H-benzo[1,4]oxazin-6-ylmethoxy]-1-(toluene-4-sulphonyl)piperidin-2-yl]-2,2-dimethylpropionic acid

Analogously to Example 17b, methyl 3-[(2S,5R)-5-[4-(3-methoxypropyl)-2-methyl-2-phenyl-3,4-dihydro-2H-benzo[1,4]oxazin-6-ylmethoxy]-1-(toluene-4-sulphonyl)piperidin-2-yl]-2,2-dimethylpropionate is reacted. The title compound is identified based on the Rf value.

### c) Methyl 3-[(2S,5R)-5-[4-(3-methoxypropyl)-2-methyl-2-phenyl-3,4-dihydro-2H-benzo[1,4]oxazin-6-ylmethoxy]-1-(toluene-4-sulphonyl)piperidin-2-yl]-2,2-dimethylpropionate

Analogously to Method F, methyl 3-[(2S,5R)-5-[4-(3-methoxypropyl)-2-methyl-3-oxo-2-phenyl-3,4-dihydro-2H-benzo[1,4]oxazin-6-ylmethoxy]-1-(toluene-4-sulphonyl)piperidin-2-yl]-2,2-dimethylpropionate is reacted. The title compound is identified based on the Rf value.

### d) Methyl 3-[(2S,5R)-5-[4-(3-methoxypropyl)-2-methyl-3-oxo-2-phenyl-3,4-dihydro-2H-benzo[1,4]oxazin-6-ylmethoxy]-1-(toluene-4-sulphonyl)piperidin-2-yl]-2,2-dimethylpropionate

Analogously to Method H, methyl 3-[(2S,5R)-5-hydroxy-1-(toluene-4-sulphonyl)piperidin-2-yl]-2,2-dimethylpropionate and 6-bromomethyl-4-(3-methoxypropyl)-2-methyl-2-phenyl-4H-benzo[1,4]oxazin-3-one are reacted. The title compound is identified based on the Rf value.

### e) 6-Bromomethyl-4-(3-methoxypropyl)-2-methyl-2-phenyl-4H-benzo[1,4]oxazin-3-one

A solution of 4.4 mmol of 4-(3-methoxypropyl)-2,6-dimethyl-2-phenyl-4H-benzo[1,4]oxazin-3-one in 50 ml of carbon tetrachloride at 70° C. is admixed with 4.4 mmol of N-bromosuccinimide. The reaction mixture is heated to 90° C. and then admixed with 0.09 mmol of 2,2′-azobis(2-methylpropionitrile) and 0.09 mmol of dibenzoyl peroxide. After 2 hours, the reaction mixture is cooled to room temperature, clarified by filtration and concentrated by evaporation. The title compound is identified from the residue by means of flash chromatography (SiO₂ 60F) based on the Rf value.

### f) 4-(3-Methoxypropyl)-2,6-dimethyl-2-phenyl-4H-benzo[1,4]oxazin-3-one

A suspension of 7.22 mmol of 2,6-dimethyl-2-phenyl-4H-benzo[1,4]oxazin-3-one, 14.45 mmol of 1-chloro-3-methoxypropane, 7.23 g of potassium fluoride on aluminium oxide and 0.14 mmol of potassium iodide in 150 ml of acetonitrile is stirred at reflux over 72 hours. The reaction mixture is cooled and clarified by filtration, and the filtrate is concentrated by evaporation. The title compound is identified from the residue by means of flash chromatography (SiO₂ 60F) based on the Rf value.

### g) 2,6-Dimethyl-2-phenyl-4H-benzo[1,4]oxazin-3-one

A solution of 6.27 mmol of methyl 2-(4-methyl-2-nitrophenoxy)-2-phenylpropionate in 26 ml of acetic acid and 1.5 ml of water is admixed at 50° C. with 36 mmol of iron powder in portions. After 4 hours, the reaction mixture is cooled to room temperature and clarified by filtration, and the filtrate is washed with brine (3×). The organic phase is dried over sodium sulphate and concentrated by evaporation. The title compound is identified from the residue by means of flash chromatography (SiO₂ 60F) based on the Rf value.

### h) Methyl 2-(4-methyl-2-nitrophenoxy)-2-phenylpropionate

A solution of 24.14 mmol of 4-methyl-2-nitrophenol [119-33-5] in 25 ml of acetonitrile at room temperature is admixed with 36.22 mmol of caesium carbonate and a solution of methyl 2-bromo-2-phenylpropionate [84892-13-7] in 10 ml of acetonitrile. After reflux for 24 hours, the reaction mixture is cooled to room temperature and clarified by filtration, and the filtrate is concentrated by evaporation. The residue is dissolved in ethyl acetate and washed successively with water and brine, dried over sodium sulphate and concentrated by evaporation. The title compound is identified from the residue by means of flash chromatography (SiO₂ 60F) based on the Rf value.

The following compounds are prepared in an analogous manner by the process described in Example 44:
45 3-{(2S,5R)-5-[2-(3-Chlorophenyl)-4-(3-methoxypropyl)-2-methyl-3,4-dihydro-2H-benzo[1,4]oxazin-6-ylmethoxy]piperidin-2-yl}-2,2-dimethyl-N-(tetrahydropyran-4-ylmethyl)propionamide

Starting from ethyl 2-(3-chlorophenyl)-2-hydroxypropionate [198287-11-5].
46 3-{(2S,5R)-5-[2-(3-Fluorophenyl)-4-(3-methoxypropyl)-2-methyl-3,4-dihydro-2H-benzo[1,4]oxazin-6-ylmethoxy]piperidin-2-yl}-2,2-dimethyl-N-(tetrahydropyran-4-ylmethyl)propionamide

Starting from ethyl (3-fluorophenyl)oxoacetate [110193-594].
47 3-{(2S,5R)-5-[2-(3,5-Difluorophenyl)-4-(3-methoxypropyl)-2-methyl-3,4-dihydro-2H-benzo[1,4]oxazin-6-ylmethoxy]piperidin-2-yl}-2,2-dimethyl-N-(tetrahydropyran-4-ylmethyl)propionamide

Starting from ethyl (3,5-difluorophenyl)oxoacetate [208259-57-8].
48 3-{(2S,5R)-5-[(S)-4-(3-Methoxypropyl)-2-methyl-2-phenyl-3,4-dihydro-2H-benzo[1,4]oxazin-6-ylmethoxy]piperidin-2-yl}-2,2-dimethyl-N-(tetrahydropyran-4-ylmethyl)propionamide

Starting from methyl (S)-2-hydroxy-2-phenylpropionate [13448-80-1].
49 3-{(2S,5R)-5-[(S)-2-(3-Chlorophenyl)-4-(3-methoxypropyl)-2-methyl-3,4-dihydro-2H-benzo[1,4]oxazin-6-ylmethoxy]piperidin-2-yl}-2,2-dimethyl-N-(tetrahydropyran-4-ylmethyl)propionamide

Starting from methyl (S)-2-(3-chlorophenyl)-2-hydroxypropionate.

The starting material is prepared as follows:

### a) Methyl (S)-2-(3-chlorophenyl)-2-hydroxypropionate

A solution of 0.04 mmol of (3S,5S)-1-benzyl-5-(hydroxydiphenylmethyl)-pyrrolidin-3-ol [648424-86-6] in 1 ml of toluene at room temperature is admixed with 0.036 mmol of dimethylzinc (1M in hexane), stirred for 30 minutes, admixed with isopropanol (8.25 μl in 0.1 ml of toluene), stirred for a further 30 minutes and then cooled to −20° C. The reaction mixture is admixed with 0.40 mmol of methyl (3-chlorophenyl)oxoacetate [34966-50-2] and then admixed slowly (over 30 hours) with 0.96 mmol of dimethylzinc (1M in hexane). 12 hours later, the reaction mixture is quenched with 10% aqueous citric acid solution and extracted with ethyl acetate (3×)—the combined organic phases are dried with sodium sulphate and concentrated by evaporation. The title compound is identified from the residue by means of flash chromatography (SiO₂ 60F) based on the Rf value.
50 3-{(2S,5R)-5-[(S)-2-(3-Fluorophenyl)-4-(3-methoxypropyl)-2-methyl-3,4-dihydro-2H-benzo[1,4]oxazin-6-ylmethoxy]piperidin-2-yl}-2,2-dimethyl-N-(tetrahydropyran-4-ylmethyl)propionamide

Starting from methyl (S)-2-(3-fluorophenyl)-2-hydroxypropionate, which is prepared analogously to Example 49a from methyl (3-fluorophenyl)oxoacetate [185030-42-6].
51 3-{(2S,5R)-5-[(S)-2-(3,5-Difluorophenyl)-4-(3-methoxypropyl)-2-methyl-3,4-dihydro-2H-benzo[1,4]oxazin-6-ylmethoxy]piperidin-2-yl}-2,2-dimethyl-N-(tetrahydropyran-4-ylmethyl)propionamide

Starting from methyl (S)-2-(3,5-difluorophenyl)-2-hydroxypropionate, which is prepared analogously to Example 49a from methyl (3,5-difluorophenyl)oxoacetate [259739-92-9].
52 (S)-3-{(2S,5R)-5-[(S)-4-(3-Methoxypropyl)-2-methyl-3-oxo-2-phenyl-3,4-dihydro-2H-benzo[1,4]oxazin-6-ylmethoxy]piperidin-2-yl}-2,2-dimethyl-N-(tetrahydropyran-4-ylmethyl)propionamide

Analogously to Example 48, omitting step c.
113 3-{(2S,5R)-5-[(S)-4-(3-Methoxy-propyl)-2-methyl-2-pyridin-4-yl-3,4-dihydro-2H-benzo[1,4]oxazin-6-ylmethoxy]-piperidin-2-yl}-2,2-dimethyl-N-(tetrahydropyran-4-ylmethyl)-propionamide

Starting from (S)-2-hydroxy-2-pyridin-4-yl-propionic acid ethyl ester, synthesized in analogy to Example 49a using oxo-pyridin-4-yl-acetic acid ethyl ester [156093-78-6].
114 3-{(2S,5R)-5-[(S)-4-(3-Methoxy-propyl)-2-methyl-2-(tetrahydro-pyran-4-yl)-3,4-dihydro-2H-benzo[1,4]oxazin-6-ylmethoxy]-piperidin-2-yl}-2,2-dimethyl-N-(tetrahydro-pyran-4-ylmethyl)-propionamide

Starting from (S)-2-hydroxy-2-(tetrahydro-pyran-4-yl)-propionic acid ethyl ester, synthesized in analogy to Example 49a using oxo-(tetrahydro-pyran-4-yl)-acetic acid ethyl ester [861160-58-9].
115 3-{(2S,5R)-5-[(R-2-Methoxymethyl-4-(3-methoxy-propyl)-2-methyl-3,4-dihydro-2H-benzo[1,4]oxazin-6-ylmethoxy]-piperidin-2-yl}-2,2-dimethyl-N-(tetrahydro-pyran-4-ylmethyl)-propionamide

Starting from (S)-2-hydroxy-3-methoxy-2-methyl-propionic acid methyl ester, synthesized in analogy to Example 49a using 3-methoxy-2-oxo-propionic acid methyl ester [8936444-3].

### Example 53

### (R(or S))-3-{(2S,5R)-5-[4-(3-Methoxypropyl)-3,4-dihydro-2H-benzo[1,4]oxazin-6-ylmethoxy]piperidin-2-yl}2-methyl-N-(tetrahydropyran-4-ylmethyl)propionamide

Following Method A, 0.436 mmol of (R(or S))-3-[(2S,5R)-5-[4-(3-methoxypropyl)-3,4-dihydro-2H-benzo[1,4]oxazin-6-ylmethoxy]-1-(toluene-4-sulphonyl)piperidin-2-yl]-2-methyl-N-(tetrahydropyran-4-ylmethyl)propionamide is reacted. The title compound is obtained as a yellow resin. Rf=0.27 (dichloromethane-methanol-conc ammonia 200:20:1); Rt=11.75 (gradient II).

The starting materials are prepared as follows:

### a) (R(or S))-3-[(2S,5R)-5-[4-(3-Methoxypropyl)-3,4-dihydro-2H-benzo[1,4]oxazin-6-ylmethoxy]-1-(toluene-4-sulphonyl)piperidin-2-yl]-2-methyl-N-(tetrahydropyran-4-ylmethyl)propionamide

Following Method B, 0.865 mmol of (R(or S))-3-[(2S,5R)-5-[4-(3-methoxypropyl)-3,4-dihydro-2H-benzo[1,4]oxazin-6-ylmethoxy]-1-(toluene-4-sulphonyl)piperidin-2-yl]-2-methylpropionic acid is reacted. The title compound is obtained as a yellow resin. Rf=0.19 (EtOAc); Rt=19.05 (gradient II).

### b) (R(or S))-3-[(2S,5R)-5-[4-(3-Methoxypropyl)-3,4-dihydro-2H-benzo[1,4]oxazin-6-ylmethoxy]-1-(toluene-4-sulphonyl)piperidin-2-yl]-2-methylpropionic acid

A solution of 0.81 mmol of methyl (R,S)-3-[(2S,5R)-5-[4-(3-methoxypropyl)-3,4-dihydro-2H-benzo[1,4]oxazin-6-ylmethoxy]-1-(toluene-4-sulphonyl)piperidin-2-yl]-2-methylpropionate in 10 ml of methanol at room temperature is admixed with 10 ml of an aqueous sodium hydroxide solution (2M) and the mixture is heated to 65° C. After 3 hours, the reaction mixture is cooled to room temperature and the methanol is concentrated by evaporation—the residue is acidified to pH 2 with 4N HCl and extracted with ethyl acetate (3×). The combined organic phases are washed with brine, dried over sodium sulphate and concentrated by evaporation. The title compound is obtained from the residue as a brown resin. Rt=19.60 (gradient II).

### c) Methyl (R,S)-3-[(2S,5R)-5-[4-(3-methoxypropyl)-3,4-dihydro-2H-benzo[1,4]oxazin-6-ylmethoxy]-1-(toluene-4-sulphonyl)piperidin-2-yl]-2-methylpropionate

A solution of 0.89 mmol of methyl 3-[(2S,5R)-5-[4-(3-methoxypropyl)-3,4-dihydro-2H-benzo[1,4]oxazin-6-ylmethoxy]-1-(toluene-4-sulphonyl)piperidin-2-yl]propionate in 20 ml of tetrahydrofuran at −78° C. is admixed over 10 minutes with sodium bis(trimethylsilyl)amide (1M in tetrahydrofuran). After one hour, the reaction mixture is admixed with 2.05 mmol of iodomethane at −78° C. After one hour, the reaction mixture is quenched with 0.5N HCl at −78° C., warmed to room temperature and extracted with dichloromethane (2×)—the combined organic phases are washed with brine, dried over sodium sulphate and concentrated by evaporation. The title compound is obtained from the residue by means of flash chromatography (SiO₂ 60F) as a yellow resin. Rf=0.40 (2:1 EtOAc-heptane); Rt=5.32 (gradient I).

### d) Methyl 3-[(2S,5R)-5-[4-(3-methoxypropyl)-3,4-dihydro-2H-benzo[1,4]oxazin-6-ylmethoxy]-1-(toluene-4-sulphonyl)piperidin-2-yl]propionate

A solution of 2.38 mmol of 3-[(2S,5R)-5-[4-(3-methoxypropyl)-3,4-dihydro-2H-benzo[1,4]oxazin-6-ylmethoxy]-1-(toluene-4-sulphonyl)piperidin-2-yl]propionic acid in 40 ml of methanol at 0° C. is admixed slowly with 22.8 mmol of trimethylsilyldiazomethane (2M in hexane). Further trimethylsilyldiazomethane (4 mmol) is added after 2, 16 and 18 hours within 15 minutes in each case. After a total of 20 hours, the reaction mixture is quenched with magnesium sulphate heptahydrate, stirred for one hour and filtered, and the filtrate is concentrated by evaporation. The title compound is obtained from the residue by means of flash chromatography (SiO₂ 60F) as a yellowish oil. Rf=0.33 (2:1 EtOAc-heptane); Rt=5.13 (gradient I).

### e) 3-[(2S,5R)-5-[4-(3-Methoxypropyl)-3,4-dihydro-2H-benzo[1,4]oxazin-6-ylmethoxy]-1-(toluene-4-sulphonyl)piperidin-2-yl]propionic acid

Analogously to Method C, 2.79 mmol of 3-[(2R,5R)-5-[4-(3-methoxypropyl)-3,4-dihydro-2H-benzo[1,4]oxazin-6-ylmethoxy]-1-(toluene-4-sulphonyl)piperidin-2-yl]-propionitrile are reacted. The title compound is obtained as a brown resin. Rf=0.37 (10:1 dichloromethane-methanol); Rt=4.54 (gradient I).

### f) 3-[(2R,5R)-5-[4-(3-Methoxypropyl)-3,4-dihydro-2H-benzo[1,4]oxazin-6-ylmethoxy]-1-(toluene-4-sulphonyl)piperidin-2-yl]propionitrile

Analogously to Method D, 2.73 mmol of 2-[(2S,5R)-5-[4-(3-methoxypropyl)-3,4-dihydro-2H-benzo[1,4]oxazin-6-ylmethoxy]-1-(toluene-4-sulphonyl)piperidin-2-yl]ethyl methanesulphonate are reacted. The title compound is obtained as an orange-brown oil. Rf=0.12 (1:1 EtOAc-heptane); Rt=4.94 (gradient I).

### g) 2-[(2S,5R)-5-[4-(3-Methoxypropyl)-3,4-dihydro-2H-benzo[1,4]oxazin-6-ylmethoxy]-1-(toluene-4-sulphonyl)piperidin-2-yl]ethyl methanesulphonate

Analogously to Method E, 2.72 mmol of 2-[(2S,5R)-5-[4-(3-methoxypropyl)-3,4-dihydro-2H-benzo[1,4]oxazin-6-ylmethoxy]-1-(toluene-4-sulphonyl)piperidin-2-yl]-ethanol are reacted. The title compound is obtained as a brown oil. Rf=0.21 (2:1 EtOAc-heptane); Rt=4.91 (gradient I).

### h) 2-[(2S,5R)-5-[4-(3-Methoxypropyl)-3,4-dihydro-2H-benzo[1,4]oxazin-6-ylmethoxy]-1-(toluene-4-sulphonyl)piperidin-2-yl]ethanol

Analogously to Example 1f, 4.63 mmol of [(2S,5R)-5-[4-(3-methoxypropyl)-3,4-dihydro-2H-benzo[1,4]oxazin-6-ylmethoxy]-1-(toluene-4-sulphonyl)piperidin-2-yl]-acetaldehyde (Example 1f) are reacted. The title compound is obtained as a yellow resin. Rf=0.37 (EtOAc); Rt=4.54 (gradient I).

### Example 54

### (S(or R))-3-{(2S,5R)-5-[4-(3-Methoxypropyl)-3,4-dihydro-2H-benzo[1,4]oxazin-6-ylmethoxy]piperidin-2-yl}-2-methyl-N-(tetrahydropyran-4-ylmethyl)propionamide

Analogously to Method A, 0.066 mmol of (S(or R))-3-[(2S,5R)-5-[4-(3-methoxypropyl)-3,4-dihydro-2H-benzo[1,4]oxazin-6-ylmethoxy]-1-(toluene-4-sulphonyl)piperidin-2-yl]-2-methyl-N-(tetrahydropyran-4-ylmethyl)propionamide is reacted. The title compound is obtained as a yellow oil. Rf=0.25 (200:20:1 dichloromethane-methanol-25% conc. ammonia); Rt=3.13 (gradient I).

The starting materials are prepared as follows:

### a) (S(or R))-3-[(2S,5R)-5-[4-(3-Methoxypropyl)-3,4-dihydro-2H-benzo[1,4]oxazin-6-ylmethoxy]-1-(toluene-4-sulphonyl)piperidin-2-yl]-2-methyl-N-(tetrahydropyran-4-ylmethyl)propionamide

Analogously to Example 17a, 0.087 mmol of (S(or R))-3-[(2S,5R)-5-[4-(3-methoxypropyl)-3,4-dihydro-2H-benzo[1,4]oxazin-6-ylmethoxy]-1-(toluene-4-sulphonyl)piperidin-2-yl]-2-methylpropionic acid is reacted. The title compound is obtained as a yellow oil. Rf=0.13 (EtOAc); Rt=4.65 (gradient I).

### b) (S(or R))-3-[(2S,5R)-5-[4-(3-Methoxypropyl)-3,4-dihydro-2H-benzo[1,4]oxazin-6-ylmethoxy]-1-(toluene-4-sulphonyl)piperidin-2-yl]-2-methylpropionic acid

To a solution of 0.069 mmol of (S)-4-benzyl-3{(S(or R))-3-[(2S,5R)-5-[4-(3-methoxypropyl)-3,4-dihydro-2H-benzo[1,4]oxazin-6-ylmethoxy]-1-(toluene-4-sulphonyl)piperidin-2-yl]-2-methylpropionyl}oxazolidin-2-one in 4 ml of tetrahydrofuran and 0.5 ml of water at 0° C. is added 0.152 mmol of lithium hydroxide, followed by 0.3 ml of 30% hydrogen peroxide. The reaction mixture is stirred at 0° C. for 2 hours, then quenched with 10 ml of saturated aqueous sodium thiosulphate, and stirred at room temperature for 30 minutes. 0.1N HCl is added till pH=2. The reaction mixture is extracted with dichloromethane (3×), dried over sodium sulphate and concentrated to afford the title compound as a light brown oil. Rf=0.12 (EtOAc-heptane 2:1); Rt=4.66 (gradient I).

### c) (S)-4-Benzyl-3-{(S(or R))-3-[(2S,5R)-5-[4-(3-methoxypropyl)-3,4-dihydro-2H-benzo[1,4]oxazin-6-ylmethoxy]-1-(toluene-4-sulphonyl)piperidin-2-yl]-2-methylpropionyl}oxazolidin-2-one

Following Method F, 0.468 mmol of 6-[(3R,6S)-6-[(S(or R))-3-((S)-4-benzyl-2-oxo-oxazolidin-3-yl)-2-methyl-3-oxopropyl]-1-(toluene-4-sulphonyl)piperidin-3-yloxymethyl]-4-(3-methoxypropyl)-4H-benzo[1,4]oxazin-3-one is reacted. The title compound is obtained as a white solid. Rf=0.45 (EtOAc-heptane 2:1); Rt=5.79 (gradient I).

### d) 6-[(3R,6S)-6-[(S(or R))-3-((S)-4-Benzyl-2-oxooxazolidin-3-yl)-2-methyl-3-oxopropyl]-1-(toluene-4-sulphonyl)piperidin-3-yloxymethyl]-4-(3-methoxypropyl)-4H-benzo[1,4]oxazin-3-one

A solution of 0.799 mmol of (S)-4-benzyl-3-{(S(or R))-3-[(2S,5R)-5-hydroxy-1-(toluene-4-sulphonyl)piperidin-2-yl]-2-methylpropionyl}oxazolidin-2-one and 0.959 mmol of 2,2,2-trichloroacetimidic acid 4-(3-methoxypropyl)-3-oxo-3,4-dihydro-2H-benzo[1,4]oxazin-6-ylmethyl ester in 10 ml of dichloromethane at 0° C. is treated with 0.040 mmol of scandium triflate and stirred at 0° C. for 2 hours. The reaction mixture is then diluted with 30 ml of dichloromethane and washed with 30 ml of water. The aqueous layer is re-extracted with dichloromethane (2×20 ml). The combined organic extracts are washed with brine, dried over sodium sulphate and concentrated. The crude product is purified by flash chromatography (SiO₂ 60F) to afford the title compound as a yellow solid. Rf=0.42 (EtOAc-heptane 2:1); Rt=5.49 (gradient I).

### e) (S)-4-Benzyl-3-{(S(or R))-3-[(2S,5R)-5-hydroxy-1-(toluene-4-sulphonyl)piperidin-2-yl]-2-methylpropionyl}oxazolidin-2-one

A solution of 1.128 mmol of (S)-4-benzyl-3-{(S(or R))-3-[(2S,5R)-5-(4-methoxybenzyloxy)-1-(toluene-4-sulphonyl)piperidin-2-yl]-2-methylpropionyl}oxazolidin-2-one and 0.113 mmol of 5% palladium on charcoal in 3 ml of methanol and 2 ml of tetrahydrofuran is hydrogenated at atmospheric pressure for 18 h. The reaction mixture is then filtered through Hyflo, concentrated and purified by flash chromatography (SiO₂ 60F) to afford the title compound as a beige solid. Rf=0.31 (EtOAc-heptane 2:1); Rt=4.64 (gradient I).

### f) (S)-4-Benzyl-3-{(S(or R))-3-[(2S,5R)-5-(4-methoxybenzyloxy)-1-(toluene-4-sulphonyl)piperidin-2-yl]-2-methylpropionyl}oxazolidin-2-one

To a solution of 2.241 mmol of (S)-4-benzyl-3-{3-[(2S,5R)-5-(4-methoxybenzyloxy)-1-(toluene-4-sulphonyl)piperidin-2-yl]propionyl}oxazolidin-2-one in 14 ml of tetrahydrofuran at −78° C. is added 2.689 mmol of lithium diisopropylamide. The reaction mixture is stirred at −78° C. for 45 minutes, then at 40° C. for 20 minutes. The mixture is cooled again to −78° C. for 10 minutes, before the addition of 11.205 mmol of methyl iodide. The reaction mixture is stirred at −78° C. for 1 hour, then at −40° C. for 18 hours. It is quenched at −40° C. with saturated aqueous ammonium chloride. Once at room temperature, it is partitioned between dichloromethane and saturated aqueous ammonium chloride. The aqueous phase is re-extracted with dichloromethane (3×). The combined organic extracts are dried over sodium sulphate, concentrated and purified by flash chromatography (SiO₂ 60F) to afford the title compound as a white solid. Rf=0.55 (EtOAc-heptane); Rt=5.74 (gradient I).

### g) (S)-4-Benzyl-3-{3-[(2S,5R)-5-(4-methoxybenzyloxy)-1-(toluene-4-sulphonyl)piperidin-2-yl]propionyl}oxazolidin-2-one

A solution of 5.541 mmol of 3-[(2S,5R)-5-(4-methoxybenzyloxy)-1-(toluene-4-sulphonyl)piperidin-2-yl]propionic acid and 6.095 mmol of triethylamine in 20 ml of diethyl ether and 3 ml of dichloromethane is cooled to −78° C. 5.541 mmol of pivaloyl chloride are added, the reaction mixture is stirred at −78° C. for 5 minutes, then at 0° C. for 1 hour, then cooled again to −78° C. In the meantime, 6.649 mmol of (S)-benzyl-2-oxazolidinone are dissolved in 20 ml of tetrahydrofuran and cooled to −78° C. 6.372 mmol of n-butyllithium (1.6M in hexane) are added dropwise, and the mixture is stirred at −78° C. for 45 minutes. The solution of the mixed anhydride previously mentioned is transferred via cannula, washing with 5 ml of tetrahydrofuran. The reaction mixture is stirred at −78° C. for 1 h, then transferred in an ice-bath and stirred for 1.5 hours, then at room temperature for 0.5 h. It is quenched with 1N ammonium chloride, extracted with dichloromethane (4×), dried over sodium sulphate, and concentrated. Purification by flash chromatography (SiO₂ 60F) affords the title compound as a white solid. Rf=0.51 (EtOAc-heptane 2:1); Rt=5.26 (gradient I).

### h) 3-[(2S,5R)-5-(4-Methoxybenzyloxy)-1-(toluene-4-sulphonyl)piperidin-2-yl]-propionic acid

Following Example 17b, 4.116 mmol of 3-[(2S,5R)-5-(4-methoxybenzyloxy)-1-(toluene-4-sulphonyl)piperidin-2-yl]propionic acid methyl ester (Example 23 g) are reacted. The title compound is obtained as a colourless sticky oil. Rf=0.24 (EtOAc-heptane 2.1); Rt=4.48 (gradient I).

### i) 2,2,2-Trichloroacetimidic acid 4-(3-methoxypropyl)-3-oxo-3,4-dihydro-2H-benzo[1,4]oxazin-6-ylmethyl ester

To a solution of 3.98 mmol of 6-hydroxymethyl-4-(3-methoxypropyl)-4H-benzo[1,4]oxazin-3-one [857272-03-8] in 15 ml of dichloromethane at 0° C. is added 15 ml of 50% aqueous KOH, followed by 0.199 mmol of tetrabutylammonium hydrogen sulphate, and 4.776 mmol of trichloroacetonitrile. The reaction mixture is stirred vigorously at 0° C. for 1 hour, then at room temperature for 1 hour. It is then extracted with dichloromethane (3×). The combined organic extracts are dried over sodium sulphate, and concentrated, to afford the title compound as a yellow gum. Rf=0.50 (EtOAc-heptane 1:1).

The following compounds are prepared in an analogous manner by the processes described in Examples 53 and 54:
56 (S(or R))-3-{(2S,5R)-5-[4-(3-Methoxypropyl)-3,4-dihydro-2H-benzo[1,4]oxazin-6-ylmethoxy]piperidin-2-yl}-2-methyl-N-(tetrahydropyran-4-yl)propionamide
59 (R(or S))—N-(2-Carbamoyl-2-methylpropyl)-3-{(2S,5R)-5-[4-(3-methoxypropyl)-3,4-dihydro-2H-benzo[1,4]oxazin-6-ylmethoxy]piperidin-2-yl}-2-methylpropionamide
62 (S(or R))-3-{(2S,5R)-5-[4-(3-Methoxypropyl)-3-oxo-3,4-dihydro-2H-benzo[1,4]oxazin-6-ylmethoxy]piperidin-2-yl}-2-methyl-N-(tetrahydropyran-4-ylmethyl)propionamide
63 (R(or S))-3-{(2S,5R)-5-[2-(3-Chlorophenyl)-4-(3-methoxypropyl)-2-methyl-3,4-dihydro-2H-benzo[1,4]oxazin-6-ylmethoxy]piperidin-2-yl}-2-methyl-N-(tetrahydropyran-4-ylmethyl)propionamide

Starting from ethyl 2-(3-chlorophenyl)-2-hydroxypropionate [198287-11-5].
64 (R(or S))-3-{(2S,5R)-5-[2-(3-Fluorophenyl)-4-(3-methoxypropyl)-2-methyl-3,4-dihydro-2H-benzo[1,4]oxazin-6-ylmethoxy]piperidin-2-yl}-2-methyl-N-(tetrahydropyran-4-ylmethyl)propionamide

Starting from ethyl (3-fluorophenyl)oxoacetate [110193-594].
68 (S(or R))-3-{(2S,5R)-5-[2-(3,5-Difluorophenyl)-4-(3-methoxypropyl)-2-methyl-3,4-dihydro-2H-benzo[1,4]oxazin-6-ylmethoxy]piperidin-2-yl}-2-methyl-N-(tetrahydropyran-4-ylmethyl)propionamide

Starting from ethyl (3,5-difluorophenyl)oxoacetate [208259-57-8].
79 (R(or S))-3-{(2S,5R)-5-[4-Methoxy-3-(3-methoxypropoxy)benzyloxy]piperidin-2-yl}-2-methyl-N-(tetrahydropyran-4-ylmethyl)propionamide

Starting from 4-bromomethyl-1-methoxy-2-(3-methoxypropoxy)benzene [172900-73-1].
80 (R(or S))-2-{(2S,5R)-5-[4-(3-Methoxypropyl)-3,4-dihydro-2H-benzo[1,4]oxazin-6-ylmethoxy]piperidin-2-ylmethyl}-N-(tetrahydropyran-4-ylmethyl)butyramide

Starting from methyl 3-[(2S,5R)-5-[4-(3-methoxypropyl)-3,4-dihydro-2H-benzo[1,4]oxazin-6-ylmethoxy]-1-(toluene-4-sulphonyl)piperidin-2-yl]propionate (Example 53d) using ethyl iodide instead of methyl iodide (in the step analogously to Example 53c).

### Example 84

### 1-(2-{(2S,5R)-5-[4-(3-Methoxypropyl)-3,4-dihydro-2H-benzo[1,4]oxazin-6-ylmethoxy]piperidin-2-yl}-1,1-dimethylethyl)-3-(tetrahydropyran-4-yl)urea

Analogously to Method A, 1-{2-[(2S,5R)-5-[4-(3-methoxypropyl)-3,4-dihydro-2H-benzo[1,4]oxazin-6-ylmethoxy]-1-(toluene-4-sulphonyl)piperidin-2-yl]-1 , 1-dimethylethyl}-3-(tetrahydropyran-4-yl)urea is reacted. The title compound is identified based on the Rf value.

The starting materials are prepared as follows:

### a) 1-{2-[(2S,5R)-5-[4-(3-Methoxypropyl)-3,4-dihydro-2H-benzo[1,4]oxazin-6-ylmethoxy]-1-(toluene-4-sulphonyl)piperidin-2-yl]-1, -dimethylethyl}-3-(tetrahydropyran-4-yl)urea

A solution of 0.221 mmol of 6-[(3R,6S)-6-(2-isocyanato-2-methylpropyl)-1-(toluene-4-sulphonyl)piperidin-3-yloxymethyl]-4-(3-methoxypropyl)-3,4-dihydro-2H-benzo[1,4]oxazine and 2.21 mmol of 4-aminotetrahydropyran in 1 ml of tetrahydrofuran is stirred at room temperature for 2 hours. The reaction mixture is concentrated by evaporation. The title compound is identified from the residue by means of flash chromatography (SiO₂ 60F) based on the Rf value.

### b) 6-[(3R,6S)-6-(2-isocyanato-2-methylpropyl)-1-(toluene-4-sulphonyl)piperidin-3-yloxymethyl]-4-(3-methoxypropyl)-3,4-dihydro-2H-benzo[1,4]oxazine

A solution of 0.398 mmol of 3-[(2S,5R)-5-[4-(3-methoxypropyl)-3,4-dihydro-2H-benzo[1,4]oxazin-6-ylmethoxy]-1-(toluene-4-sulphonyl)piperidin-2-yl]-2,2-dimethylpropionic acid (Example 17b) and 0.995 mmol of triethylamine in 4 ml of tetrahydrofuran is cooled to 0° C. and 0.796 mmol of ethyl chloroformate is added. The reaction mixture is stirred at 0° C. for one hour and then a solution of 7.96 mmol of sodium azide in 2 ml of water is added at 0° C. The reaction solution is stirred at 0° C. for 45 minutes. The mixture is diluted with water and ethyl acetate—the aqueous phase is washed with water (2×), dried with sodium sulphate and concentrated by evaporation. The residue is taken up in 2 ml of toluene and heated to 115° C. for 2 hours. The reaction mixture is cooled to room temperature and concentrated by evaporation. The crude title compound is obtained from the residue.

### Example 87

### 4-{(2S,5R)-5-[4-(3-Methoxypropyl)-3,4-dihydro-2H-benzo[1,4]oxazin-6-ylmethoxy]piperidin-2-yl}-3,3-dimethyl-1-(tetrahydropyran-4-ylamino)butan-2-one

Analogously to Method A, 4-[(2S,5R)-5-[4-(3-methoxypropyl)-3,4-dihydro-2H-benzo[1,4]oxazin-6-ylmethoxy]-1-(toluene-4-sulphonyl)piperidin-2-yl]-3,3-dimethyl-1-(tetrahydropyran-4-ylamino)butan-2-one is reacted. The title compound is identified based on the Rf value.

The starting materials are prepared as follows:

### a) 4-[(2S,5R)-5-[4-(3-Methoxypropyl)-3,4-dihydro-2H-benzo[1,4]oxazin-6-ylmethoxy]-1-(toluene-4-sulphonyl)piperidin-2-yl]-3,3-dimethyl-1-(tetrahydropyran-4-ylamino)butan-2-one

A solution of 0.6 mmol of tetrahydropyran-4-ylamine in 5 ml of diethyl ether at room temperature is admixed with a solution of 0.2 mmol of 1-chloro-4-[(2S,5R)-5-[4-(3-methoxypropyl)-3,4-dihydro-2H-benzo[1,4]oxazin-6-ylmethoxy]-1-(toluene-4-sulphonyl)piperidin-2-yl]-3,3-dimethylbutan-2-one in 5 ml of diethyl ether. After 8 hours, the reaction mixture is admixed with 15% NaOH—the organic phase is washed successively with water and brine, dried over sodium sulphate and concentrated by evaporation. The title compound is identified from the residue by means of flash chromatography (SiO₂ 60F) based on the Rf value.

### b) 1-Chloro-4-[(2S,5R)-5-[4-(3-methoxypropyl)-3,4-dihydro-2H-benzo[1,4]oxazin-6-ylmethoxy]-1-(toluene-4-sulphonyl)piperidin-2-yl]-3,3-dimethylbutan-2-one

A solution of 2.24 mmol of chloroiodomethane in 20 ml of tetrahydrofuran and 20 ml of diethyl ether at −100° C. is admixed with 1.12 mmol of n-butyllithium (1.6M in hexane). After one hour, 0.32 mmol of methyl 3-[(2S,5R)-5-[4-(3-methoxypropyl)-3,4-dihydro-2H-benzo[1,4]oxazin-6-ylmethoxy]-1-(toluene-4-sulphonyl)piperidin-2-yl]-2,2-dimethylpropionate (Example 17c) is added. The reaction mixture is heated to −78° C. over one hour, poured into brine and extracted with tert-butyl methyl ether (3×)—the combined organic phases are washed with brine, dried over sodium sulphate and concentrated by evaporation. The title compound is identified from the residue by means of flash chromatography (SiO₂ 60F) based on the Rf value.

### Example 88

### N-(2-{(2S,5R)-5-[4-(3-Methoxypropyl)-3,4-dihydro-2H-benzo[1,4]oxazin-6-ylmethoxy]piperidin-2-yl}-1,1-dimethylethyl)-2-(tetrahydropyran-4-yl)acetamide

Following Method A, 0.163 mmol of N-{2-[(2S,5R)-5-[4-(3-methoxypropyl)-3,4-dihydro-2H-benzo[1,4]oxazin-6-ylmethoxy]-1-(toluene-4-sulphonyl)piperidin-2-yl]-1,1-dimethylethyl}-2-(tetrahydropyran-4-yl)acetamide is reacted to afford the title compound as a yellow resin. Rf=0.17 (dichloromethane-methanol-conc. ammonia); Rt=3.34 (gradient I).

The starting materials are prepared as follows:

### a) N-{2-[(2S,5R)-5-[4-(3-Methoxypropyl)-3,4-dihydro-2H-benzo[1,4]oxazin-6-ylmethoxy]-1-(toluene-4-sulphonyl)piperidin-2-yl]-1,1-dimethylethyl}-2-(tetrahydropyran-4-yl)acetamide

A solution of 0.511 mmol of tetrahydropyranyl-4-acetic acid [85064-61-5] in 5 ml of dichloromethane is treated with 1.023 mmol of 1-chloro-N,N-2-trimethylpropenylamine. The reaction mixture is stirred at room temperature for 1.5 hours. In a second flask, a solution of 0.341 mmol of 2-[(2S,5R)-5-[4-(3-methoxypropyl)-3,4-dihydro-2H-benzo[1,4]oxazin-6-ylmethoxy]-1-(toluene-4-sulphonyl)piperidin-2-yl]-1,1-dimethylethylamine in 10 ml of dichloromethane are treated with 1.023 mmol of triethylamine, and cooled to 0° C. The solution of acid chloride is added dropwise to this second flask, and the reaction mixture is stirred at room temperature for 2 hours. Water is added, and the aqueous phase is extracted with dichloromethane (3×). The combined organic extracts are dried over sodium sulphate, concentrated and purified by flash chromatography (SiO₂ 60F) to afford the title compound as a dark yellow resin. Rf=0.20 (dichloromethane-methanol-conc ammonia); Rt=4.94 (gradient I).

### b) 2-[(2S,5R)-5-[4-(3-Methoxypropyl)-3,4-dihydro-2H-benzo[1,4]oxazin-6-ylmethoxy]-1-(toluene-4-sulphonyl)piperidin-2-yl]-1,1-dimethylethylamine

A solution of 1.383 mmol of {2-[(2S,5R)-5-[4-(3-methoxypropyl)-3,4-dihydro-2H-benzo[1,4]oxazin-6-ylmethoxy]-1-(toluene-4-sulphonyl)piperidin-2-yl]-1,1-dimethylethyl}carbamic acid benzyl ester and 0.138 mmol of palladium on charcoal in 20 ml of methanol is hydrogenated at atmospheric pressure and room temperature for 2 hours. The reaction mixture is then filtered through Hyflo, and concentrated under reduced pressure, to afford the title compound as a yellow resin. Rf=0.17 (dichloromethane-methanol-conc ammonia); Rt=4.22 (gradient I).

### c) {2-[(2S,5R)-5-[4-(3-Methoxypropyl)-3,4-dihydro-2H-benzo[1,4]oxazin-6-ylmethoxy]-1-(toluene-4-sulphonyl)piperidin-2-yl]-1,1-dimethylethyl}carbamic acid benzyl ester

A mixture of 1.484 mmol of 6-[(3R,6S)-6-(2-isocyanato-2-methylpropyl)-1-(toluene-4-sulphonyl)piperidin-3-yloxymethyl]-4-(3-methoxypropyl)-3,4-dihydro-2H-benzo[1,4]oxazine (Example 84a) and 29.68 mmol of benzyl alcohol is stirred at 120° C. for 2 hours then cooled to room temperature. Purification by flash chromatography (SiO₂ 60F) affords the title compound as a yellow resin. Rf=0.27 (EtOAc-heptane 1:1); Rt=5.71 (gradient I).

### Example 89

### N—((R(or S))-1-{(2S,5R)-5-[4-(3-Methoxypropyl)-3,4-dihydro-2H-benzo[1,4]oxazin-6-ylmethoxy]piperidin-2-ylmethyl}-2-methylpropyl)-2-(tetrahydropyran-4-yl)acetamide

Following Method A, 0.066 mmol of N-{(R(or S))-1-[(2S,5R)-5-[4-(3-methoxypropyl)-3,4-dihydro-2H-benzo[1,4]oxazin-6-ylmethoxy]-1-(toluene-4-sulphonyl)piperidin-2-ylmethyl]-2-methylpropyl}-2-(tetrahydropyran-4-yl)acetamide is reacted to afford the title compound as a yellow oil. Rf=0.27 (dichloromethane-methanol conc ammonia 200:20:1); Rt=3.26 (gradient I).

The starting materials are prepared as follows:

### a) N-{(R(or S))-1-[(2S,5R)-5-[4-(3-Methoxypropyl)-3,4-dihydro-2H-benzo[1,4]oxazin-6-ylmethoxy]-1-(toluene-4-sulphonyl)piperidin-2-ylmethyl]-2-methylpropyl}-2-(tetrahydropyran-4-yl)acetamide

Following the procedure for Example 88a, 0.393 mmol of (R(or S))-1-[(2S,5R)-5-[4-(3-methoxypropyl)-3,4-dihydro-2H-benzo[1,4]oxazin-6-ylmethoxy]-1-(toluene-4-sulphonyl)piperidin-2-ylmethyl]-2-methylpropylamine is reacted. The title compound is obtained as a yellow oil. Rf=0.46 (dichloromethane-methanol-conc ammonia 200:20:1); Rt=4.77 (gradient I).

### b) (R(or S))-1-[(2S,5R)-5-[4-(3-Methoxypropyl)-3,4-dihydro-2H-benzo[1,4]oxazin-6-ylmethoxy]-1-(toluene-4-sulphonyl)piperidin-2-ylmethyl]-2-methylpropylamine

Following Method O, 0.393 mmol of 6-[(3R,6S)-6-((R(or S))-2-azido-3-methylbutyl)-1-(toluene-4-sulphonyl)piperidin-3-yloxymethyl]-4-(3-methoxypropyl)-3,4-dihydro-2H-benzo[1,4]oxazine is reacted to afford the title compound as a brown oil. Rf=0.60 (EtOAc); Rt=4.29 (gradient I).

### c) 6-[(3R,6S)-6-((R(or S))-2-Azido-3-methylbutyl)-1-(toluene-4-sulphonyl)piperidin-3-yloxymethyl]-4-(3-methoxypropyl)-3,4-dihydro-2H-benzo[1,4]oxazine

A solution of 0.412 mmol of methanesulphonic acid (S(or R))-1-[(2S,5R)-5-[4-(3-methoxypropyl)-3,4-dihydro-2H-benzo[1,4]oxazin-6-ylmethoxy]-1-(toluene-4-sulphonyl)piperidin-2-ylmethyl]-2-methylpropyl ester and 2.06 mmol of sodium azide is stirred at 80° C. for 18 hours. The reaction mixture is cooled to room temperature, diluted with water, extracted with tert-butyl methyl ether (3×). The combined organic extracts are washed with water, then brine, dried over sodium sulphate, and concentrated. The crude product is obtained as a brown oil. Rf=0.47 (EtOAc-heptane 2.1); Rt=5.95 (gradient I).

### d) Methanesulphonic acid (S(or R))-1-[(2S,5R)-5-[4-(3-methoxypropyl)-3,4-dihydro-2H-benzo[1,4]oxazin-6-ylmethoxy]-1-(toluene-4-sulphonyl)piperidin-2-ylmethyl]-2-methylpropyl -ester

Following Method E, 0.412 mmol of (S(or R))-1-[(2S,5R)-5-[4-(3-methoxypropyl)-3,4-dihydro-2H-benzo[1,4]oxazin-6-ylmethoxy]-1-(toluene-4-sulphonyl)piperidin-2-yl]-3-methylbutan-2-ol is reacted to afford the title compound as a brown oil. Rf=0.40 (EtOAc-heptane 2:1); Rt=5.27 (gradient I).

### e) (i) (S(or R))-1-[(2S,5R)-5-[4-(3-Methoxypropyl)-3,4-dihydro-2H-benzo[1,4]oxazin-6-ylmethoxy]-1-(toluene-4-sulphonyl)piperidin-2-yl]-3-methylbutan-2-ol and

### (ii) (R(or S))-1-[(2S,5R)-5-[4-(3-Methoxypropyl)-3,4-dihydro-2H-benzo[1,4]oxazin-6-ylmethoxy]-1-(toluene-4-sulphonyl)piperidin-2-yl]-3-methylbutan-2-ol

To a solution of 1.56 mmol of [(2S,5R)-5-[4-(3-methoxypropyl)-3,4-dihydro-2H-benzo[1,4]oxazin-4-ylmethoxy]-1-(toluene-4-sulphonyl)piperidin-2-yl]acetaldehyde (1f) in 10 ml of dry tetrahydrofuran at −15° C. is added 3.12 mmol of isopropylmagnesium chloride. The reaction mixture is stirred between −15° C. and 0° C. for 3 hours, then at room temperature for 1 hour. It is quenched with saturated aqueous ammonium chloride and extracted with dichloromethane (3×). The combined organic extracts are dried over sodium sulphate, concentrated and purified by flash chromatography (SiO₂ 60F) to afford the title compounds as colourless oils. Rf=0.24 and 0.20 (EtOAc-heptane 1:1); Rt=5.34 and 5.20 (gradient I).

In analogy to Examples 89 and 40, the following compounds are synthesized:
100 N—((R(or S))-1-{(2S,5R)-5-[4-(3-Methoxypropyl)-2,2-dimethyl-3,4-dihydro-2H-benzo[1,4]oxazin-6-ylmethoxy]piperidin-2-ylmethyl}-2-methylpropyl)-2-(tetrahydropyran-4-yl)acetamide
102 4-Methoxycyclohexanecarboxylic acid ((R(or S))-1-{(2S,5R)-5-[4-(3-methoxypropyl)-2,2-dimethyl-3,4-dihydro-2H-benzo[1,4]oxazin-6-ylmethoxy]piperidin-2-ylmethyl}-2-methylpropyl)amide

### Example 99

### N—((S(or R))-2-{(2S,5R)-5-[4-(3-Methoxypropyl)-2,2-dimethyl-3,4-dihydro-2H-benzo[1,4]oxazin-6-ylmethoxy]piperidin-2-yl}-1-methylethyl)-2-(tetrahydropyran-4-yl)acetamide

Following Method A, N—{(S(or R))-2-[(2S,5R)-5-[4-(3-methoxypropyl)-2,2-dimethyl-3,4-dihydro-2H-benzo[1,4]oxazin-6-ylmethoxy]-1-(toluene-4-sulphonyl)piperidin-2-yl]-1-methylethyl}-2-(tetrahydropyran-4-yl)-acetamide is reacted. The title compound is identified based on the Rf value.

The starting materials are prepared as follows:

### a) N-{(S(or R))-2-[(2S,5R)-5-[4-(3-methoxypropyl)-2,2-dimethyl-3,4-dihydro-2H-benzo[1,4]oxazin-6-ylmethoxy]-1-(toluene-4-sulphonyl)piperidin-2-yl]-1-methylethyl}-2-(tetrahydropyran-4-yl)acetamide

Following the procedure for Example 88a, (S(or R))-2-[(2S,5R)-5-[4-(3-methoxypropyl)-2,2-dimethyl-3,4-dihydro-2H-benzo[1,4]oxazin-6-ylmethoxy]-1-(toluene-4-sulphonyl)piperidin-2-yl]-1-methylethylamine is reacted. The title compound is identified based on the Rf value.

### b) (S(or R))-2-[(2S,5R)-5-[4-(3-Methoxypropyl)-2,2-dimethyl-3,4-dihydro-2H-benzo[1,4]oxazin-6-ylmethoxy]-1-(toluene-4-sulphonyl)piperidin-2-yl]-1-methylethylamine

Following Method O, 6-[(3R,6S)-6-((S(or R))-2-azidopropyl)-1-(toluene-4-sulphonyl)piperidin-3-yloxymethyl]-4-(3-methoxypropyl)-2,2-dimethyl-3,4-dihydro-2H-benzo[1,4]oxazine is reacted. The title compound is identified based on the Rf value.

### c) 6-[(3R,6S)-6-((S(or R))-2-azidopropyl)-1-(toluene-4-sulphonyl)piperidin-3-yloxymethyl]-4-(3-methoxypropyl)-2,2-dimethyl-3,4-dihydro-2H-benzo[1,4]oxazine

Following the procedure for Example 89c, methanesulphonic acid (R(or S))-2-[(2S,5R)-5-[4-(3-methoxypropyl)-2,2-dimethyl-3,4-dihydro-2H-benzo[1,4]oxazin-6-ylmethoxy]-1-(toluene-4-sulphonyl)piperidin-2-yl]-1-methylethyl ester is reacted. The title compound is identified based on the Rf value.

### d) Methanesulphonic acid (R(or S))-2-[(2S,5R)-5-[4-(3-methoxypropyl)-2,2-dimethyl-3,4-dihydro-2H-benzo[1,4]oxazin-6-ylmethoxy]-1-(toluene-4-sulphonyl)piperidin-2-yl]-1-methylethyl ester

Following Method E, (R(or S))-1-[(2S,5R)-5-[4-(3-methoxypropyl)-2,2-dimethyl-3,4-dihydro-2H-benzo[1,4]oxazin-6-ylmethoxy]-1-(toluene-4-sulphonyl)piperidin-2-yl]propan-2-ol is reacted. The title compound is identified based on the Rf value.

### e) (i) (R(or S))-1-[(2S,5R)-5-[4-(3-methoxypropyl)-2,2-dimethyl-3,4-dihydro-2H-benzo[1,4]oxazin-6-ylmethoxy]-1-(toluene-4-sulphonyl)piperidin-2-yl]Propan-2-ol and

### (ii) (S(or R))-1-[(2S,5R)-5-[4-(3-methoxypropyl)-2,2-dimethyl-3,4-dihydro-2H-benzo[1,4]oxazin-6-ylmethoxy]-1-(toluene-4-sulphonyl)piperidin-2-yl]propan-2-ol

Following the procedure for Example 89e, [(2S,5R)-5-[4-(3-methoxypropyl)-2,2-dimethyl-3,4-dihydro-2H-benzo[1,4]oxazin-6-ylmethoxy]-1-(toluene-4-sulphonyl)piperidin-2-yl]acetaldehyde is reacted with methylmagnesium bromide. The title compounds are identified based on the Rf values.

### f) [(2S,5R)-5-[4-(3-methoxypropyl)-2,2-dimethyl-3,4-dihydro-2H-benzo[1,4]oxazin-6-ylmethoxy]-1-(toluene-4-sulphonyl)piperidin-2-yl]-acetaldehyde

The title compound is prepared in analogy to the synthesis of Example 1f, starting from 6-chloromethyl-4-(3-methoxypropyl)-2,2-dimethyl-4H-benzo[1,4]oxazin-3-one [857281-71-1]

In analogy to the synthesis of Example 99, the following compound is prepared:
101 4-Methoxycyclohexanecarboxylic acid ((S(or R))-2-{(2S,5R)-5-[4-(3-methoxypropyl)-2,2-dimethyl-3,4-dihydro-2H-benzo[1,4]oxazin-6-ylmethoxy]piperidin-2-yl}-1-methylethyl)amide
104 Tetrahydro-pyran-4-carboxylic acid ((S(or R))-2-{(2S,5R)-5-[4-(3-methoxypropyl)-2,2-dimethyl-3,4-dihydro-2H-benzo[1,4]oxazin-6-ylmethoxy]-piperidin-2-yl}-1-methyl-ethyl)-amide

### Example 103

### 4-(3-Methoxy-propyl)-2,2-dimethyl-6-[(3R,6S)-6-(2-methyl-2-[1,2,4]triazol-4-yl-propyl)-piperidin-3-yloxymethyl]-3,4-dihydro-2H-benzo[1,4]oxazine

Following general Method A, 4-(3-methoxy-propyl)-2,2-dimethyl-6-[(3R,6S)-6-(2-methyl-2-[1,2,4]triazol-4-yl-propyl)-1-(toluene-4-sulfonyl)piperidin-3-yloxymethyl]-3,4-dihydro-2H-benzo[1,4]oxazine is reacted. The title compound is identified based on the Rf value.

The starting materials are prepared as follows:

### a) 4-(3-Methoxy-propyl)-2,2-dimethyl-6-[(3R,6S)-6-(2-methyl-2-[1.2.4]triazol-4-yl-propyl)-1-(toluene-4-sulfonyl)piperidin-3-yloxymethyl]-3,4-dihydro-2H-benzo[1,4]oxazine

A solution of 0.70 mmol of 4H-[1,2,4]triazole in 1 ml N,N-dimethylformamide is treated at room temperature with 0.70 mmol of sodium hydride (60% dispersion in oil) and stirred for 30 minutes. A solution of 0.14 mmol of methanesulfonic acid 2-[(2S,5R)-5-[4-(3-methoxy-propyl)-2,2-dimethyl-3,4-dihydro-2H-benzo[1,4]oxazin-6-ylmethoxy]-1-(toluene-4-sulfonyl)-piperidin-2-yl]-1,1-dimethyl-ethyl ester in 1 ml N,N-dimethylformamide is added and the reaction mixture is warmed at 40° C. After 24 hours, the reaction mixture is cooled to room temperature, diluted with water, and extracted with ethyl acetate (3×). The combined organic extracts are dried with sodium sulfate, and concentrated. The residue is purified by flash chromatography (SiO₂ 60F) to afford the title compound, which is identified based on the Rf value.

### b) Methanesulfonic acid 2-[(2S,5R)-5-[4-(3-methoxy-propyl)-2,2-dimethyl-3,4-dihydro-2H-benzo[1,4]oxazin-6-ylmethoxy]-1-(toluene-4-sulfonyl)-piperidin-2-yl]-1,1-dimethyl-ethyl ester

Following general Method E, 1-[(2S,5R)-5-[4-(3-methoxy-propyl)-2,2-dimethyl-3,4-dihydro-2H-benzo[1,4]oxazin-6-ylmethoxy]-1-(toluene-4-sulfonyl)-piperidin-2-yl]-2-methyl-propan-2-ol is reacted. The title compound is identified based on the Rf value.

### c) 1-[(2S,5R)-5-[4-(3-methoxy-propyl)-2,2-dimethyl-3,4-dihydro-2H-benzo[1,4]oxazin-6-ylmethoxy]-1-(toluene-4-sulfonyl)-piperidin-2-yl]-2-methyl-propan-2-ol

Following the procedure for Example 15b, 1-[(2S,5R)-5-[4-(3-methoxy-propyl)-2,2-dimethyl-3,4-dihydro-2H-benzo[1,4]oxazin-6-ylmethoxy]-1-(toluene-4-sulfonyl)-piperidin-2-yl]-propan-2-one is reacted. The title compound is identified based on the Rf value.

### d) 1-[(2S,5R)-5-[4-(3-methoxy-propyl)-2,2-dimethyl-3,4-dihydro-2H-benzo[1,4]oxazin-6-ylmethoxy]-1-(toluene-4-sulfonyl)-piperidin-2-yl]-propan-2-one

Following the procedure for Example 15c, (S,R)-1-[(2S,5R)-5-[4-(3-methoxy-propyl)-2,2-dimethyl-3,4-dihydro-2H-benzo[1,4]oxazin-6-ylmethoxy]-1-(toluene-4-sulfonyl)-piperidin-2-yl]-propan-2-ol (Example 99e) is reacted. The title compound is identified based on the Rf value.

### Example 105

### (S(or R))-2-Methoxy-3-{(2S,5R)-5-[4-(3-methoxy-propyl)-2,2-dimethyl-3,4-dihydro-2H-benzo[1,4]oxazin-6-ylmethoxy]-piperidin-2-yl}-N-(tetrahydro-pyran-4-ylmethyl) propionamide

Following general Method A, (S(or R))-2-methoxy-3-[(2S,5R)-5-[4-(3-methoxypropyl)-2,2-dimethyl-3,4-dihydro-2H-benzo[1,4]oxazin-6-ylmethoxy]-1-(toluene-4-sulfonyl)-piperidin-2-yl]-N-(tetrahydro-pyran-4-ylmethyl)-propionamide is reacted.

The title compound is identified based on the Rf value.

The starting materials are prepared as follows:

### a) (S(or R))-2-Methoxy-3-[(2S,5R)-5-[4-(3-methoxy-propyl)-2,2-dimethyl-3,4-dihydro-2H-benzo[1,4]oxazin-6-ylmethoxy]-1-(toluene-4-sulfonyl)-piperidin-2-yl]-N-(tetrahydro-pyran-4-ylmethyl)-propionamide

Following the procedure for Example 17a, (S(or R))-2-methoxy-3-[(2S,5R)-5-[4-(3-methoxy-propyl)-2,2-dimethyl-3,4-dihydro-2H-benzo[1,4]oxazin-6-ylmethoxy]-1-(toluene-4-sulfonyl)-piperidin-2-yl]-propionic acid is reacted. The title compound is identified based on the Rf value.

### b) (S(or R))-2-Methoxy-3-[(2S,5R)-5-[4-(3-methoxy-propyl)-2,2-dimethyl-3,4-dihydro-2H-benzo[1,4]oxazin-6-ylmethoxy]-1-(toluene-4-sulfonyl)-piperidin-2-yl]-propionic acid

Following the procedure for Example 54b, (S)-4-benzyl-3-{(S(or R))-2-methoxy-3-[(2S,5R)-5-[4-(3-methoxy-propyl)-2,2-dimethyl-3,4-dihydro-2H-benzo[1,4]oxazin-6-ylmethoxy]-1-(toluene-4-sulfonyl)-piperidin-2-yl]-propionyl}-oxazolidin-2-one is reacted. The title compound is identified based on the Rf value.

### c) (S)-4-Benzyl-3-{(S(or R))-2-methoxy-3-[(2S,5R)-5-[4-(3-methoxy-propyl)-2,2-dimethyl-3,4-dihydro-2H-benzo[1,4]oxazin-6-ylmethoxy]-1-(toluene-4-sulfonyl)-piperidin-2-yl]-propionyl}-oxazolidin-2-one

Following the procedure for Example 54c, 6-[(3R,6S)-6-[(S(or R))-3-((S)-4-Benzyl-2-oxo-oxazolidin-3-yl)-2-methoxy-3-oxo-propyl]-1-(toluene-4-sulfonyl)-piperidin-3-yloxymethyl]-4-(3-methoxy-propyl)-2,2-dimethyl-4H-benzo[1,4]oxazin-3-one is reacted. The title compound is identified based on the Rf value.

### d) 6-[(3R,6S)-6-[(S(or R))-3-((S)-4-Benzyl-2-oxo-oxazolidin-3-yl)-2-methoxy-3-oxo-propyl]-1-(toluene-4-sulfonyl)-piperidin-3-yloxymethyl]-4-(3-methoxy-propyl)-2,2-dimethyl-4H-benzo[1,4]oxazin-3-one

Following the procedure for Example 54d, (S)-4-Benzyl-3-{(S)-3-[(2S,5R)-5-hydroxy-1-(toluene-4-sulfonyl)-piperidin-2-yl]-2-methoxy-propionyl}-oxazolidin-2-one and 2,2,2-trichloro-acetimidic acid 4-(3-methoxy-propyl)-2,2-dimethyl-3-oxo-3,4-dihydro-2H-benzo[1,4]oxazin-6-ylmethyl ester are reacted. The title compound is identified based on the Rf value.

### e) (S)-4-Benzyl-3-{(S)-3-[(2S,5R)-5-hydroxy-1-(toluene-4-sulfonyl)-piperidin-2-yl]-2-methoxy-propionyl}-oxazolidin-2-one

Following the procedure for Example 54e, (S)-4-benzyl-3-{(S)-2-methoxy-3-[(2S,5R)-5-(4-methoxy-benzyloxy)-1-(toluene-4-sulfonyl)-piperidin-2-yl]-propionyl}-oxazolidin-2-one is reacted. The title compound is identified based on the Rf value.

### f) (S)-4-Benzyl-3-{(S)-2-methoxy-3-[(2S,5R)-5-(4-methoxy-benzyloxy)-1-(toluene-4-sulfonyl)-piperidin-2-yl]-propionyl}-oxazolidin-2-one

To a solution of 0.2 mmol of (S)-4-benzyl-3-{(S)-2-hydroxy-3-[(2S,5R)-5-(4-methoxybenzyloxy)-1-(toluene-4-sulfonyl)-piperidin-2-yl]-propionyl}-oxazolidin-2-one in 10 ml of dry dichloromethane is added sequentially at 0° C. 4 Å molecular sieves (500 mg), N,N,N′,N′-tetramethyl-naphthalene-1,8-diamine (2 mmol) and trimethyloxonium tetrafluoroborate (1.8 mmol). After stirring at room temperature for 3 hours, the reaction is quenched by adding water (20 ml). The layers are separated and the aqueous phase is extracted with tert butyl-methyl ether (3×). The combined organic solutions are washed with saturated, aqueous copper sulfate (2×) and brine, dried with sodium sulfate, and concentrated under reduced pressure. The residue is purified by flash chromatography (SiO₂ 60F) to afford the title compound, which is identified based on the Rf value.

### g) (S)-4-Benzyl-3-[(S)-2-hydroxy-3-[(2S,5R)-5-(4-methoxy-benzyloxy)-1-(toluene-4-sulfonyl)-piperidin-2-yl]-propionyl]-oxazolidin-2-one

A solution of 0.25 mmol of (S)-4-benzyl-3-{3-[(2S,5R)-5-(4-methoxy-benzyloxy)-1-(toluene-4-sulfonyl)-piperidin-2-yl]-propionyl}-oxazolidin-2-one (Example 54 g) in 15 ml of tetrahydrofuran at −78° C. is treated with 0.30 mmol of sodium bis(trimethylsilyl)amide. The reaction mixture is stirred at −78° C. for 30 minutes, then at −40° C. for 15 minutes, before being cooled again to −78° C. A solution of 0.33 mmol of 3-phenyl-2-(phenylsulfonyl)-1,2-oxaziridine [63160-134] in 5 ml of tetrahydrofuran is added, the reaction mixture is stirred at −78° C. for 2 hours, then quenched with 100 ml of saturated aqueous ammonium chloride solution. Once at room temperature, the reaction mixture is extracted with tert butyl-methyl ether (3×). The combined organic extracts are washed with brine, with sodium sulfate and concentrated under reduced pressure. The residue is purified by flash chromatography (SiO₂ 60F) to afford the title compound, which is identified based on the Rf value.

### h) 2,2,2-Trichloro-acetimidic acid 4-(3-methoxy-propyl)-2,2-dimethyl-3-oxo-3,4-dihydro-2H-benzo[1,4]oxazin-6-ylmethyl ester

Following the procedure for Example 54j, 6-hydroxymethyl-4-(3-methoxy-propyl)-2,2-dimethyl-4H-benzo[1,4]oxazin-3-one [857281-72-2] is reacted. The title compound is identified based on the Rf value.

In analogy to the synthesis of Example 105, the following compound is prepared:
106 (R(or S))-2-Methoxy-3-{(2S,5R)-5-[4-(3-methoxy-propyl)-2,2-dimethyl-3,4-dihydro-2H-benzo[1,4]oxazin-6-ylmethoxy]-piperidin-2-yl}-N-(tetrahydro-pyran-4-ylmethyl)-propionamide

### Example 107

### 3-{(2S,5R)-5-[4-(3-Methoxy-propyl)-2,2-dimethyl-3,4-dihydro-2H-benzo[1,4]oxazin-6-ylmethoxy]-piperidin-2-yl}-2,2-dimethyl-N—[(S)-1-(tetrahydro-pyran-4-yl)ethyl]-propionamide

Following general Method A, 3-[(2S,5R)-5-[4-(3-methoxy-propyl)-2,2-dimethyl-3,4-dihydro-2H-benzo[1,4]oxazin-6-ylmethoxy]-1-(toluene-4-sulfonyl)-piperidin-2-yl]-2,2-dimethyl-N—[(S)-1-(tetrahydro-pyran-4-yl)-ethyl]-propionamide is reacted. The title compound is identified based on the Rf value.

The starting materials are prepared as follows:

### a) (i) 3-[(2S,5R)-5-[4-(3-Methoxy-propyl)-2,2-dimethyl-3,4-dihydro-2H-benzo[1,4]oxazin-6-ylmethoxy]-1-(toluene-4-sulfonyl)-piperidin-2-yl]-2,2-dimethyl-N—[(S)-1-(tetrahydro-pyran-4-yl)-ethyl]-propionamide and

### (ii) 3-[(2S,5R)-5-[4-(3-Methoxy-propyl)-2,2-dimethyl-3,4-dihydro-2H-benzo[1,4]oxazin-6-ylmethoxy]-1-(toluene-4-sulfonyl)-piperidin-2-yl]-2,2-dimethyl-N—[(R)-1-(tetrahydro-pyran-4-yl)-ethyl]-propionamide

Following the procedure for Example 17a, 3-[(2S,5R)-5-[4-(3-methoxy-propyl)-2,2-dimethyl-3,4-dihydro-2H-benzo[1,4]oxazin-6-ylmethoxy]-1-(toluene-4-sulfonyl)-piperidin-2-yl]-2,2-dimethyl-propionic acid and 1-(tetrahydro-pyran-4-yl)-ethylamine [854697-78-2] are reacted. The title compound is identified based on the Rf value.

### b) 3-[(2S,5R)-5-[4-(3-Methoxy-propyl)-2,2-dimethyl-3,4-dihydro-2H-benzo[1,4]oxazin-6-ylmethoxy]-1-(toluene-4-sulfonyl)-piperidin-2-yl]-2,2-dimethylpropionic acid

Following the procedure for Example 17b, 3-[(2S,5R)-5-[4-(3-methoxy-propyl)-2,2-dimethyl-3,4-dihydro-2H-benzo[1,4]oxazin-6-ylmethoxy]-1-(toluene-4-sulfonyl)-piperidin-2-yl]-2,2-dimethyl-propionic acid methyl ester is reacted. The title compound is isolated as a pale violet resin. Rf=0.31 (EtOAc); Rt=5.28 (gradient I).

### c) 3-[(2S,5R)-5-[4-(3-Methoxy-propyl)-2,2-dimethyl-3,4-dihydro-2H-benzo[1,4]oxazin-6-ylmethoxy]-1-(toluene-4-sulfonyl)-piperidin-2-yl]-2,2-dimethylpropionic acid methyl ester

Following general Method F. 3-[(2S,5R)-5-[4-(3-methoxy-propyl)-2,2-dimethyl-3-oxo-3,4-dihydro-2H-benzo[1,4]oxazin-6-ylmethoxy]-1-(toluene-4-sulfonyl)-piperidin-2-yl]-2,2-dimethyl-propionic acid methyl ester is reacted. The title compound is isolated as a yellow oil. Rf=0.53 (EtOAc-heptane 1:1); Rt=5.79 (gradient I).

### d) 3-[(2S,5R)-5-[4-(3-Methoxy-propyl)-2,2-dimethyl-3-oxo-3,4-dihydro-2H-benzo[1,4]oxazin-6-ylmethoxy]-1-(toluene-4-sulfonyl)-piperidin-2-yl]-2,2-dimethylpropionic acid methyl ester

Following general Method H. 3-[(2S,5R)-5-hydroxy-1-(toluene-4-sulfonyl)-piperidin-2-yl]-2,2-dimethyl-propionic acid methyl ester (Example 17e) and 6-chloromethyl-4-(3-methoxy-propyl)-2,2-dimethyl-4H-benzo[1,4]oxazin-3-one [857281-71-1] are reacted. The title compound is isolated as a colourless oil. Rf=0.52 (EtOAc-heptane 3:1); Rt=5.48 (gradient I).

### Example 108

### 3-{(2S,5R)-5-[4-(3-Methoxy-propyl)-2,2-dimethyl-3,4-dihydro-2H-benzo[1,4]oxazin-4-ylmethoxy]-piperidin-2-yl}-2,2-dimethyl-N-((3R,4S)-3-methyl-tetrahydro-pyran-4-ylmethyl)-propionamide

Following general Method A, 3-[(2S,5R)-5-[4-(3-methoxy-propyl)-2,2-dimethyl-3,4-dihydro-2H-benzo[1,4]oxazin-6-ylmethoxy]-1-(toluene-4-sulfonyl)-piperidin-2-yl]-2,2-dimethyl-N-((3R,4S)-3-methyl-tetrahydro-pyran-4-ylmethyl)-propionamide is reacted. The title compound is identified based on the Rf value.

The starting materials are prepared as follows:

### a) 3-[(2S,5R)-5-[4-(3-methoxy-propyl)-2,2-dimethyl-3,4-dihydro-2H-benzo[1,4]oxazin-6-ylmethoxy]-1-(toluene-4-sulfonyl)-piperidin-2-yl]-2,2-dimethyl-N-((3R,4S)-3-methyl-tetrahydro-pyran-4-ylmethyl)-propionamide

Following the procedure for Example 17a, 3-[(2S,5R)-5-[4-(3-methoxy-propyl)-2,2-dimethyl-3,4-dihydro-2H-benzo[1,4]oxazin-6-ylmethoxy]-1-(toluene-4-sulfonyl)-piperidin-2-yl]-2,2-dimethyl-propionic acid (Example 107b) and C-((3R,4S)-3-methyl-tetrahydro-pyran-4-yl)-methylamine are reacted. The title compound is identified based on the Rf value.

### b) C-((3R,4S)-3-Methyl-tetrahydro-pyran-4-yl)-methylamine

A solution of 3.1 mmol of (3S,4R)-3-methyl-tetrahydro-pyran-4-carboxylic acid amide in 20 ml of dry tetrahydrofuran at 0° C. under argon is treated with 12.4 mmol of lithium aluminium hydride. The reaction mixture is stirred at room temperature for 16 hours. It is then quenched with 2 ml of water, followed by 4 ml of 3N NaOH. The mixture is filtered through hyflow. The filtrate is diluted with water, extracted with tert butyl-methyl ether (3×). The combined organic extracts are washed with brine, dried with sodium sulfate, and concentrated under reduced pressure. The residue is purified by flash chromatography (SiO₂ 60F) to afford the title compound, which is identified based on the Rf value.

### c) (3S,4R)-3-methyl-tetrahydro-pyran-4-carboxylic acid amide

A solution of 3.5 mmol of (3S,4R)-3-methyl-tetrahydro-pyran-4-carboxylic acid and 3.5 mmol of N,N′-carbonyldiimidazole in 10 ml of dry ethyl acetate is stirred under argon at room temperature for 4 hours. It is then quenched with 10 ml of ammonium-hydroxide and the reaction mixture is stirred for further 17 hours. The phases are separated. The organic layer is washed with water (2×), dried with sodium sulfate, and concentrated under reduced pressure. The title compound is identified based on the Rf value.

### d) (3S,4R)-3-methyl-tetrahydro-pyran-4-carboxylic acid

Following the procedure for Example 54b, (R)-4-benzyl-3-((3S,4R)-3-methyl-tetrahydro-pyran-4-carbonyl)-oxazolidin-2-one is reacted. The title compound is identified based on the Rf value.

### e) (i) (R)-4-Benzyl-3-((3S,4R)-3-methyl-tetrahydro-pyran-4-carbonyl)oxazolidin-2-one and

### (ii) (R)-4-Benzyl-3-((3R,4S)-3-methyl-tetrahydro-pyran-4-carbonyl)oxazolidin-2-one

Following the procedure for Example 54 g, cis-3-methyl-tetrahydro-pyran-4-carboxylic acid and (R) -4-benzyl-oxazolidin-2-one are reacted. The title compounds are identified based on the Rf values.

### f) cis 3-methyl-tetrahydro-pyran-4-carboxylic acid

A solution of 9.8 mmol of cis-3-methyl-tetrahydro-pyran-4-carbonitrile in 50 ml of acetic acid and 10 ml of concentrated HCl is stirred at 80° C. for 3 hours, then cooled to room temperature and concentrated under reduced pressure. The residue is partitioned between water and ethyl acetate. The water phase is re-extracted with ethyl acetate. (3×). The combined organic extracts are dried with sodium sulfate, and concentrated under reduced pressure. The residue is purified by flash chromatography (SiO₂ 60F) to afford the title compound, which is identified based on the Rf value.

### g) cis-3-Methyl-tetrahydro-pyran-4-carbonitrile

To a solution of 10.5 mmol of methanesulfonic acid 3-methyl-tetrahydro-pyran-4-yl ester in 70 ml of acetonitrile is added 21 mmol of tetrabutylammonium cyanide. The reaction mixture is stirred at 80° C. for 5 hours, then cooled to room temperature, diluted with 400 ml of water, and extracted with ethyl acetate (4×). The combined organic extracts are dried with sodium sulfate, and concentrated under reduced pressure. The residue is purified by flash chromatography (SiO₂ 60F) to afford the title compound, which is identified based on the Rf value.

### h) Methanesulfonic acid cis-3-methyl-tetrahydro-pyran-4-yl ester

A solution of 10.5 mmol of (3R,4S)-3-methyl-tetrahydro-pyran-4-ol [3174-64-9] in 80 ml of dichloromethane is cooled to 0° C. and treated with 14.7 mmol of triethylamine and 11.3 mmol of methanesulfonyl chloride. The reaction mixture is stirred for 3 hours, letting the temperature warm slowly to room temperature. Water is added, and the reaction mixture is extracted with dichloromethane (3×). The combined organic extracts are dried with sodium sulfate, and concentrated under reduced pressure. The crude title compound is identified based on the Rf value.

### Example 109

### 3-{(2S,5R)-5-[4-(3-Methoxy-propyl)-2,2-dimethyl-3,4-dihydro-2H-benzo[1,4]oxazin-6-ylmethoxy]-piperidin-2-yl}-N-((3R,4R)-3-methoxy-tetrahydro-pyran-4-ylmethyl)-2,2-dimethyl-propionamide

Following Method A, 3-[(2S,5R)-5-[4-(3-methoxy-propyl)-2,2-dimethyl-3,4-dihydro-2H-benzo[1,4]oxazin-6-ylmethoxy]-1-(toluene-4-sulfonyl)-piperidin-2-yl]-N-((3R,4R)-3-methoxy-tetrahydro-pyran-4-ylmethyl)-2,2-dimethyl-propionamide is reacted. The title compound is identified based on the Rf value.

The starting materials are prepared as follows:

### a) 3-[(2S,5R)-5-[4-(3-methoxy-propyl)-2,2-dimethyl-3,4-dihydro-2H-benzo[1,4]oxazin-6-ylmethoxy]-1-(toluene-4-sulfonyl)-piperidin-2-yl]-N-((3R,4R)-3-methoxy-tetrahydro-pyran-4-ylmethyl)-2,2-dimethyl-propionamide

Following the procedure for Example 17a, 3-[(2S,5R)-5-[4-(3-methoxy-propyl)-2,2-dimethyl-3,4-dihydro-2H-benzo[1,4]oxazin-6-ylmethoxy]-1-(toluene-4-sulfonyl)-piperidin-2-yl]-2,2-dimethyl-propionic acid (Example 107b) and C-((3R,4R)-3-methoxy-tetrahydro-pyran-4-yl)-methylamine are reacted. The title compound is identified based on the Rf value.

### b) C-((3R,4R)-3-Methoxy-tetrahydro-pyran-4-yl)-methylamine

Following the procedure for Example 108b, 108c, 108d, 108e and 108f, the title compound is obtained from cis-3-methoxy-tetrahydro-pyran-4-carbonitrile. The title compound is identified based on the Rf value.

### c) cis-3-Methoxy-tetrahydro-pyran-4-carbonitrile

A solution of 8.2 mmol of cis-3-hydroxy-tetrahydro-pyran-4-carbonitrile (Tetrahedron, 1994, 50 (4), 1261) in 65 ml of tetrahydrofuran is cooled to −78° C. and treated with 9.02 mmol of n-butyllithium (0.6M in hexane). The reaction mixture is stirred at −78° C. for 45 minutes, before the addition of 16.4 mmol of trifluoro-methanesulfonic acid methyl ester. The reaction mixture is allowed to warm to −40° C. and stirred at this temperature for 4 hours, before being quenched with saturated aqueous ammonium chloride solution. The mixture is warmed to room temperature, extracted with tert butyl-methyl ether (3×). The combined organic extracts are dried with sodium sulfate, and concentrated under reduced pressure. The residue is purified by flash chromatography (SiO₂ 60F) to afford the title compound, which is identified based on the Rf value.

### Example 110

### 1-{(2S,5R)-5-[4-(3-Methoxy-propyl)-2,2-dimethyl-3,4-dihydro-2H-benzo[1,4]oxazin-6-ylmethoxy]-piperidin-2-ylmethyl}-cyclopropanecarboxylic acid (tetrahydro-pyran-4-ylmethyl)-amide

Following Method A, 1-[(2S,5R)-5-[4-(3-methoxy-propyl)-2,2-dimethyl-3,4-dihydro-2H-benzo[1,4]oxazin-6-ylmethoxy]-1-(toluene-4-sulfonyl)-piperidin-2-ylmethyl]-cyclopropanecarboxylic acid (tetrahydro-pyran-4-ylmethyl)-amide is reacted. The title compound is identified based on the Rf value.

The starting materials are prepared as follows:

### a) 1-[(2S,5R)-5-[4-(3-Methoxy-propyl)-2,2-dimethyl-3,4-dihydro-2H-benzo[1,4]oxazin-6-ylmethoxy]-1-(toluene-4-sulfonyl)-piperidin-2-ylmethyl]-cyclopropanecarboxylic acid (tetrahydro-pyran-4-ylmethyl)-amide

Following the procedure for Example 17a, 1-[(2S,5R)-5-[4-(3-methoxy-propyl)-2,2-dimethyl-3,4-dihydro-2H-benzo[1,4]oxazin-6-ylmethoxy]-1-(toluene-4-sulfonyl)-piperidin-2-ylmethyl]-cyclopropanecarboxylic acid is reacted. The title compound is identified based on the Rf value.

### b) 1-[(2S,5R)-5-[4-(3-Methoxy-propyl)-2,2-dimethyl-3,4-dihydro-2H-benzo[1,4]oxazin-6-ylmethoxy]-1-(toluene-4-sulfonyl)-piperidin-2-ylmethyl]-cyclopropanecarboxylic acid

A solution of 2.61 mmol of 1-[(2S,5R)-5-[4-(3-methoxy-propyl)-2,2-dimethyl-3,4-dihydro-2H-benzo[1,4]oxazin-6-ylmethoxy]-1-(toluene-4-sulfonyl)-piperidin-2-ylmethyl]-cyclopropanecarboxylic acid tert-butyl ester in 20 ml of dichloromethane is treated at 0° C. with 13.05 mmol of trifluoroacetic acid. The reaction mixture is stirred at room temperature for 5 hours, then diluted with water and extracted with dichloromethane (3×). The combined organic extracts are dried with sodium sulfate, and concentrated under reduced pressure. The crude title compound is identified based on the Rf value.

### c) 1-[(2S,5R)-5-[4-(3-Methoxy-propyl)-2,2-dimethyl-3,4-dihydro-2H-benzo[1,4]oxazin-6-ylmethoxy]-1-(toluene-4-sulfonyl)-piperidin-2-ylmethyl]-cyclopropanecarboxylic acid tert-butyl ester

A solution of 5.00 mmol of cyclopropanecarboxylic acid tert-butyl ester [87661-20-9] in 50 ml of tetrahydrofuran at −78° C. is treated with 5.5 mmol of lithium diisopropylamine. The reaction mixture is stirred at −78° C. for 4 hours before the addition of a solution of 6.00 mmol of 6-[(3R,6S)-6-bromomethyl-1-(toluene-4-sulfonyl)-piperidin-3-yloxymethyl]-4-(3-methoxy-propyl)-2,2-dimethyl-3,4-dihydro-2H-benzo[1,4]oxazine in 10 ml of tetrahydrofuran. The reaction is stirred at −78° C. for 1 hour, then allowed to warm to room temperature over 4 hours, and quenched with saturated aqueous ammonium chloride solution. The mixture is extracted with tert butyl-methyl ether (3×), the combined organic extracts are dried with sodium sulfate, and concentrated under reduced pressure. The residue is purified by flash chromatography (SiO₂ 60F) to afford the title compound, which is identified based on the Rf value.

### d) 6-[(3R,6S)-6-Bromomethyl-1-(toluene-4-sulfonyl)-piperidin-3-yloxymethyl]-4-(3-methoxy-propyl)-2,2-dimethyl-3,4-dihydro-2H-benzo[1,4]oxazine

Following the procedure for Example 17d, starting with [4-(3-methoxy-propyl)-2,2-dimethyl-3,4-dihydro-2H-benzo[1,4]oxazin-6-yl]-methanol (Example 40a).

### Example 111

### N-(4-Methoxy-cyclohexylmethyl)-3-{(2S,5R)-5-[4-(3-methoxy-prolyl)-2,2-dimethyl-3,4-dihydro-2H-benzo[1,4]oxazin-6-ylmethoxy]-piperidin-2-yl}-2,2-dimethylpropionamide

Following Method A, N-(4-methoxy-cyclohexylmethyl)-3-[(2S,5R)-5-[4-(3-methoxypropyl)-2,2-dimethyl-3,4-dihydro-2H-benzo[1,4]oxazin-6-ylmethoxy]-1-(toluene-4-sulfonyl)-piperidin-2-yl]-2,2-dimethyl-propionamide is reacted. The title compound is identified based on the Rf value.

The starting materials are prepared as follows:

### a) N-(4-methoxy-cyclohexylmethyl)-3-[(2S,5R)-5-[4-(3-methoxy-propyl)-2,2-dimethyl-3,4-dihydro-2H-benzo[1,4]oxazin-6-ylmethoxy]-1-(toluene-4-sulfonyl)-piperidin-2-yl]-2,2-dimethyl-propionamide

Following the procedure for Example 17a, 3-[(2S,5R)-5-[4-(3-methoxy-propyl)-2,2-dimethyl-3,4-dihydro-2H-benzo[1,4]oxazin-6-ylmethoxy]-1-(toluene-4-sulfonyl)-piperidin-2-yl]-2,2-dimethyl-propionic acid (Example 107b) and C-(4-methoxycyclohexyl)-methylamine are reacted. The title compound is identified based on the Rf value.

### b) C-(4-Methoxy-cyclohexyl)-methylamine

Following the procedure for Example 108b and 108c, the title compound is obtained from 4-methoxy-cyclohexanecarboxylic acid [73873-59-3]. The title compound is identified based on the Rf value.

### Example 112

### 4-Methoxy-cyclohexanecarboxylic acid ((S(or R))-1-{(2S,5R)-5-[4-(3-methoxy-propyl)-2,2-dimethyl-3,4-dihydro-2H-benzo[1,4]oxazin-6-ylmethoxy]-piperidin-2-ylmethyl}-propyl amide

Following Method A, 4-methoxy-cyclohexanecarboxylic acid {(S(or R))-1-[(2S,5R)-5-[4-(3-methoxy-propyl)-2,2-dimethyl-3,4-dihydro-2H-benzo[1,4]oxazin-6-ylmethoxy]-1-(toluene-4-sulfonyl)-piperidin-2-ylmethyl]-propyl}-amide is reacted. The title compound is identified based on the Rf value.

The starting materials are prepared as follows:

### a) 4-Methoxy-cyclohexanecarboxylic acid {(S(or R))-1-[(2S,5R)-5-[4-(3-methoxypropyl)-2,2-dimethyl-3,4-dihydro-2H-benzo[1,4]oxazin-6-ylmethoxy]-1-(toluene-4-sulfonyl)-piperidin-2-ylmethyl]-propyl}-amide

Following the procedure for Example 88a, (S(or R))-1-[(2S,5R)-5-[4-(3-methoxypropyl)-2,2-dimethyl-3,4-dihydro-2H-benzo[1,4]oxazin-6-ylmethoxy]-1-(toluene-4-sulfonyl)-piperidin-2-ylmethyl]-propylamine and trans-4-methoxycyclohexane-carboxylic acid [73873-61-7] are reacted. The title compound is identified based on the Rf value.

### b) (S(or R))-1-[(2S,5R)-5-[4-(3-methoxy-propyl)-2,2-dimethyl-3,4-dihydro-2H-benzo[1,4]oxazin-6-ylmethoxy]-1-(toluene-4-sulfonyl)-piperidin-2-ylmethyl]-propylamine

Following Method O, 6-[(3R,6S)-6-((S(or R))-2-azido-butyl)-1-(toluene-4-sulfonyl)-piperidin-3-yloxymethyl]-4-(3-methoxy-propyl)-2,2-dimethyl-3,4-dihydro-2H-benzo[1,4]oxazine is reacted. The title compound is identified based on the Rf value.

### c) 6-[(3R,6S)-6-((S(or R))-2-Azido-butyl)-1-(toluene-4-sulfonyl)-piperidin-3-yloxymethyl]-4-(3-methoxy-propyl)-2,2-dimethyl-3,4-dihydro-2H-benzo[1,4]oxazine

Following the procedure for Example 89c, methanesulfonic acid (R(or S))-1-[(2S,5R)-5-[4-(3-methoxy-propyl)-2,2-dimethyl-3,4-dihydro-2H-benzo[1,4]oxazin-6-ylmethoxy]-1-(toluene-4-sulfonyl)-piperidin-2-ylmethyl]-propyl ester is reacted. The title compound is identified based on the Rf value.

### d) Methanesulfonic acid (R(or S))-1-[(2S,5R)-5-[4-(3-methoxy-propyl)-2,2-dimethyl-3,4-dihydro-2H-benzo[1,4]oxazin-6-ylmethoxy]-1-(toluene-4-sulfonyl)-piperidin-2-ylmethyl]-propyl ester

Following Method E, (R(or S))-1-[(2S,5R)-5-[4-(3-methoxy-propyl)-2,2-dimethyl-3,4-dihydro-2H-benzo[1,4]oxazin-6-ylmethoxy]-1-(toluene-4-sulfonyl)-piperidin-2-yl]-butan-2-ol is reacted. The title compound is identified based on the Rf value.

### e) (i) (R(or S))-1-[(2S,5R)-5-[4-(3-methoxy-propyl)-2,2-dimethyl-3,4-dihydro-2H-benzo[1,4]oxazin-6-ylmethoxy]-1-(toluene-4-sulfonyl)-piperidin-2-yl]-butan-2-ol and

### (ii) (S(or R))-1-[(2S,5R)-5-[4-(3-methoxy-propyl)-2,2-dimethyl-3,4-dihydro-2H-benzo[1,4]oxazin-6-ylmethoxy]-1-(toluene-4-sulfonyl)-piperidin-2-yl]-butan-2-ol

Following the procedure for Example 89e, [(2S,5R)-5-[4-(3-methoxy-propyl)-3,4-dihydro-2H-benzo[1,4]oxazin-6-ylmethoxy]-1-(toluene-4-sulfonyl)-piperidin-2-yl]-acetaldehyde (Example 1f) and ethylmagnesium bromide are reacted. The title compounds are identified based on the Rf values.

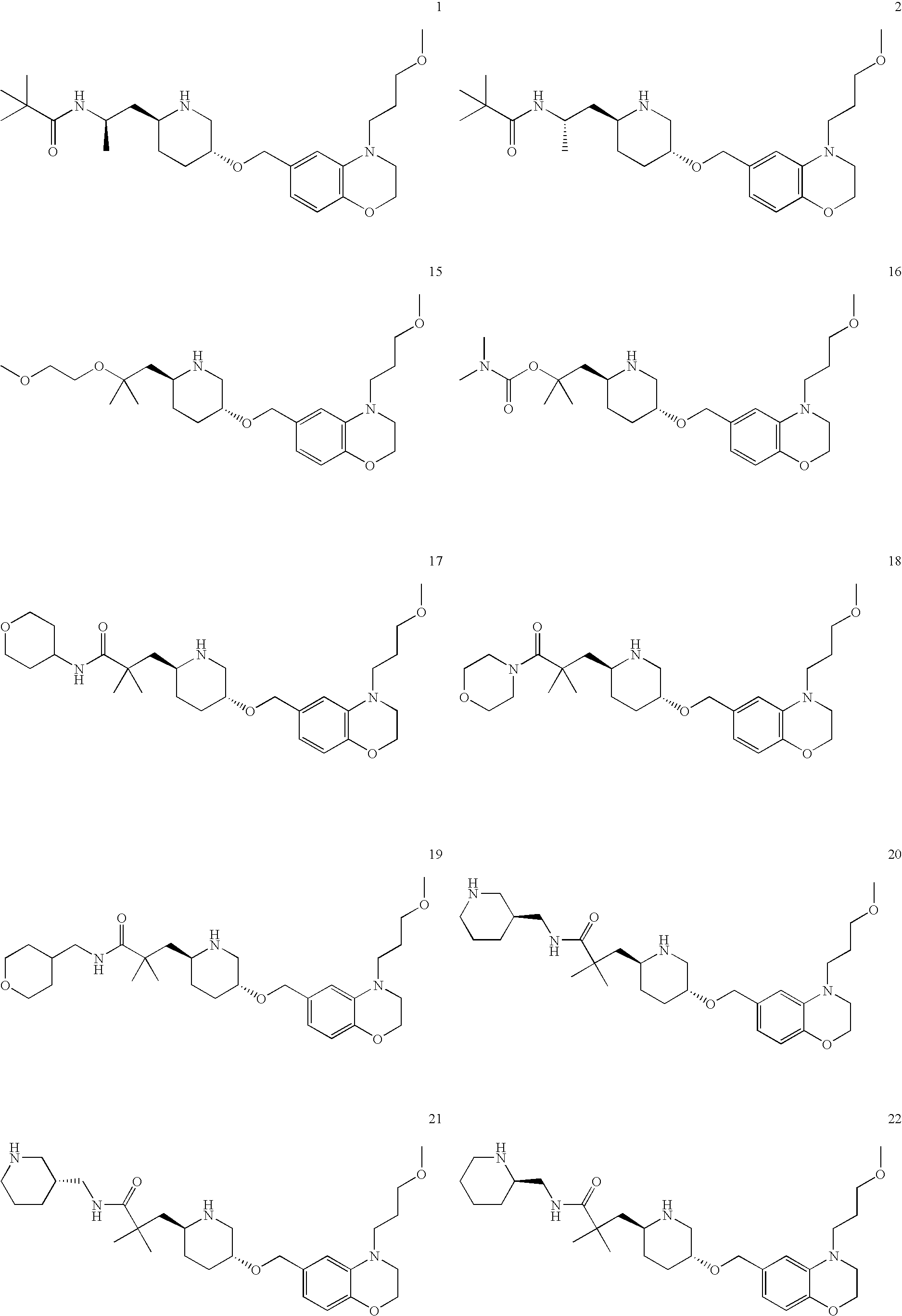

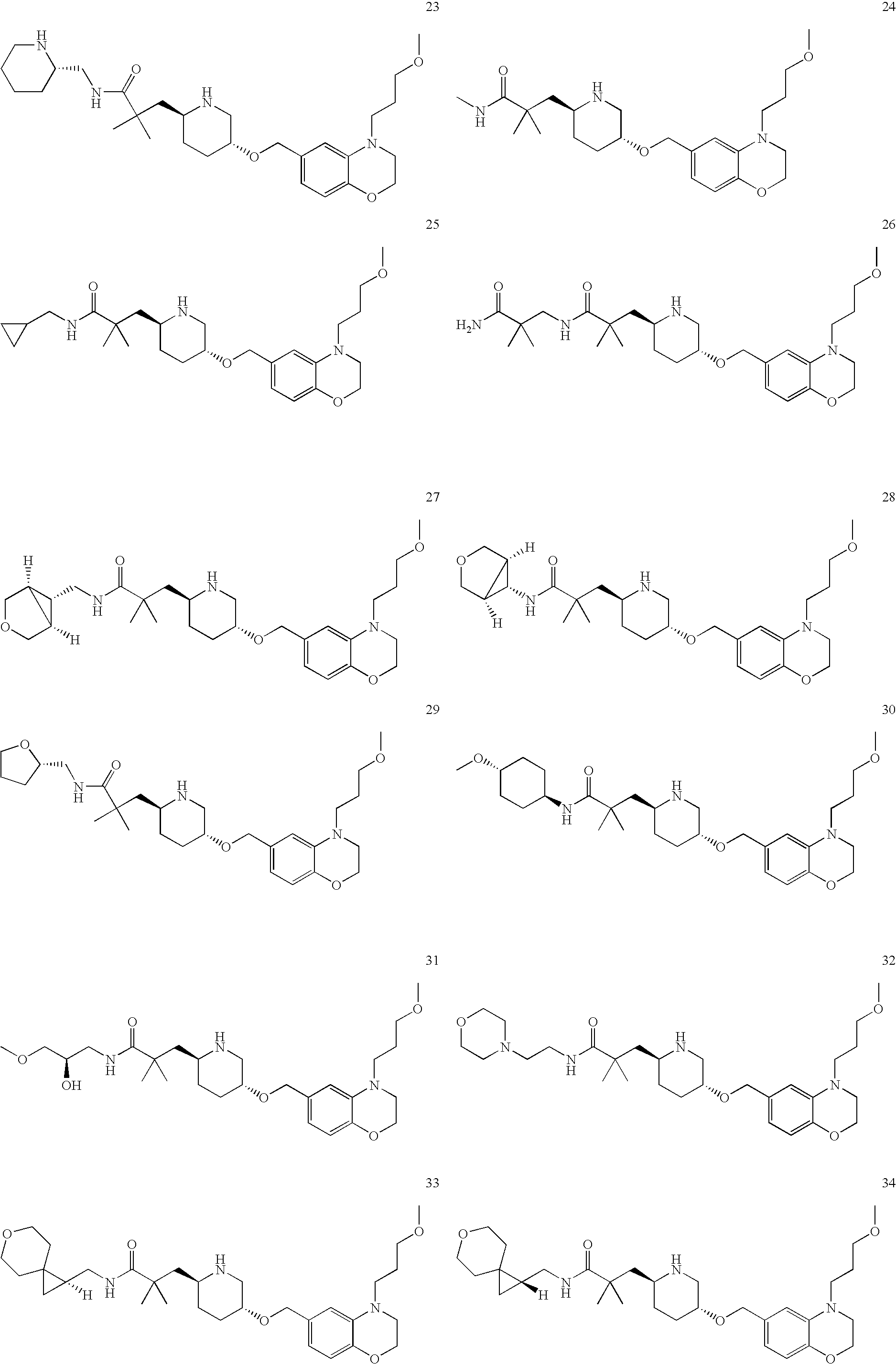

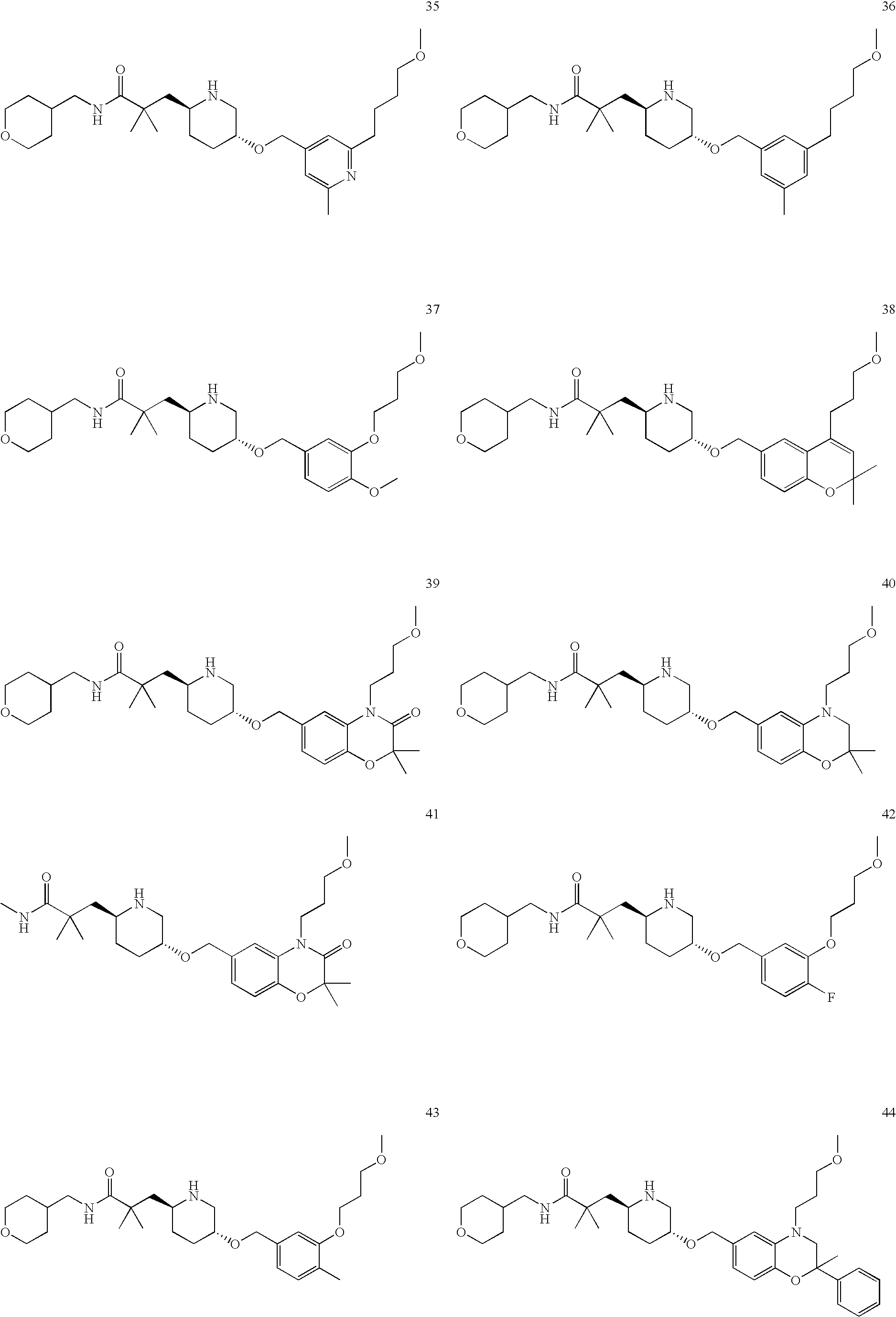

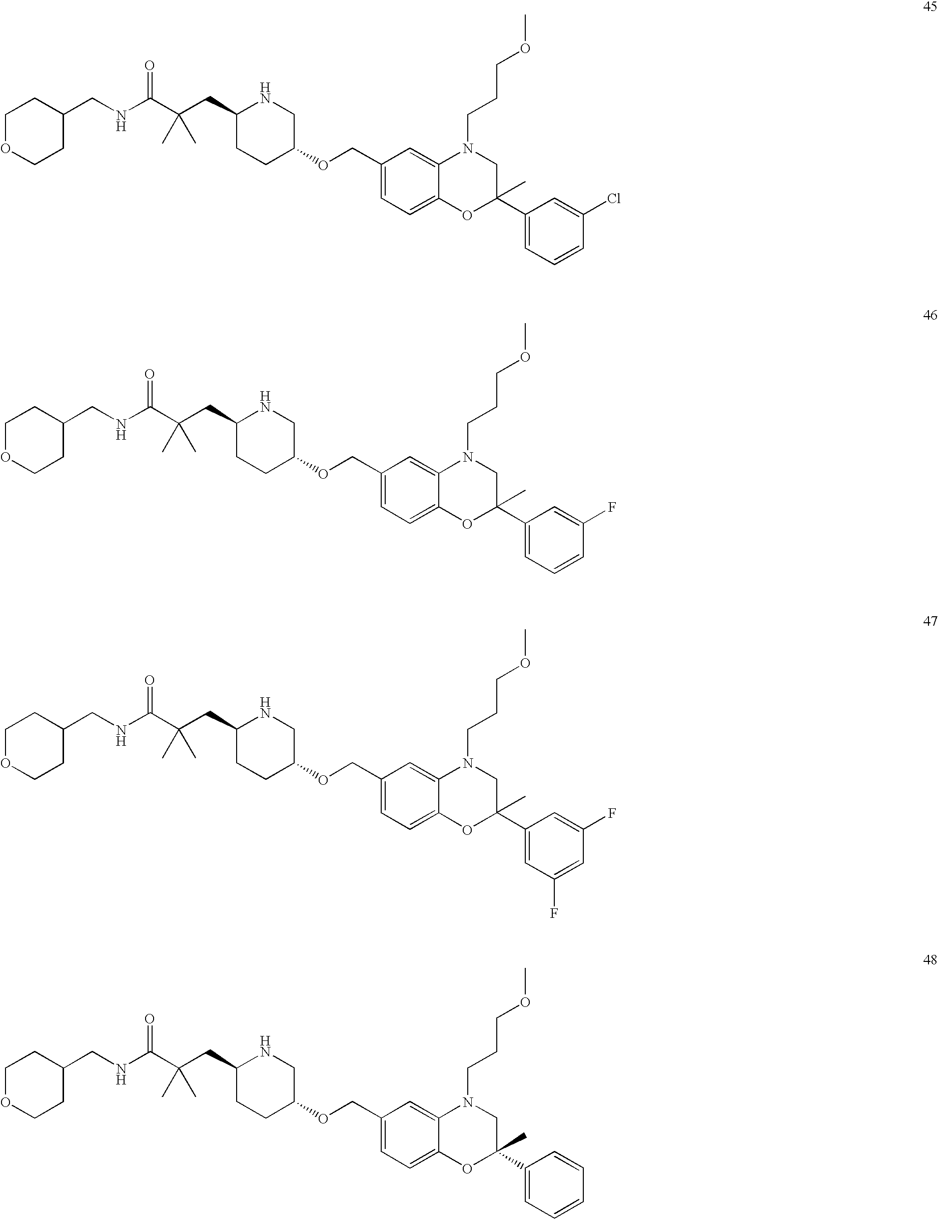

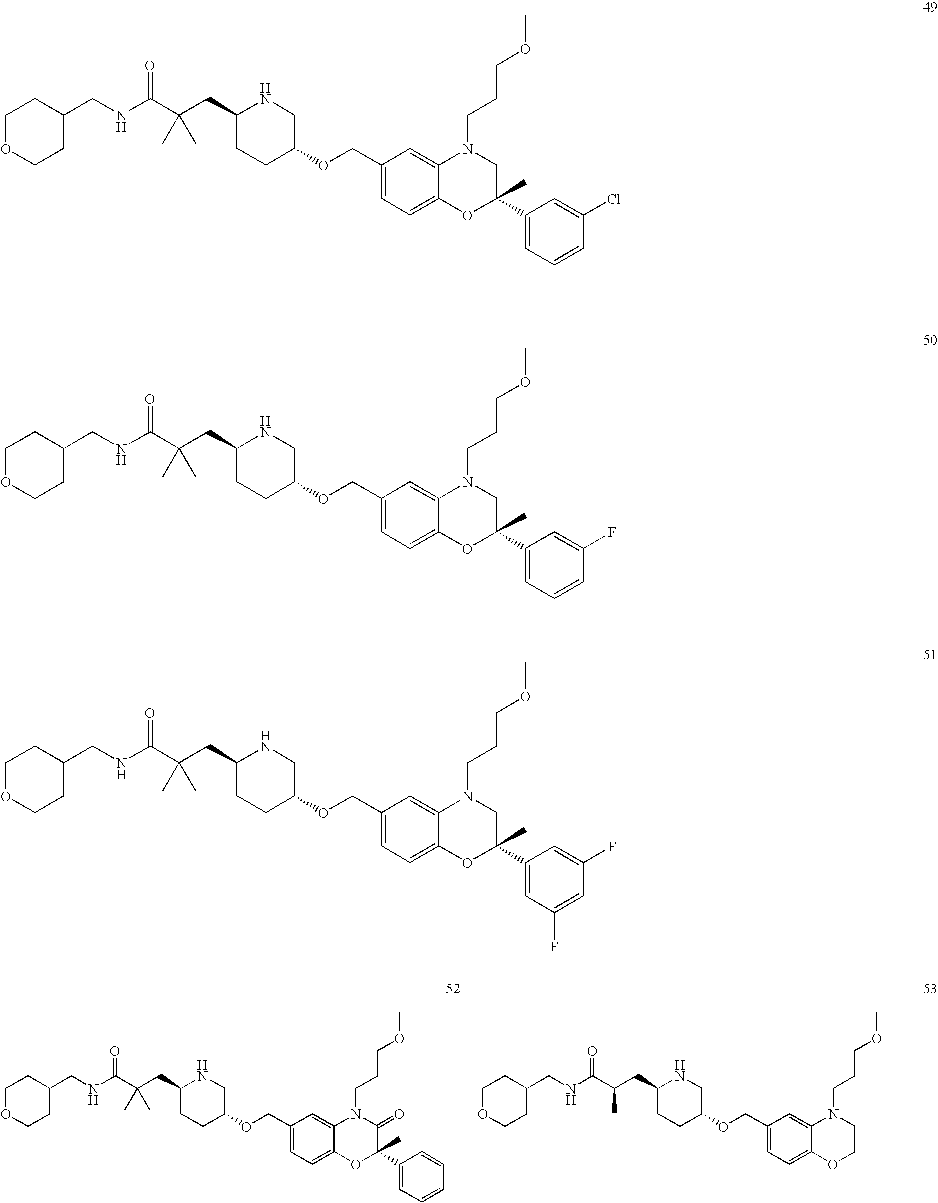

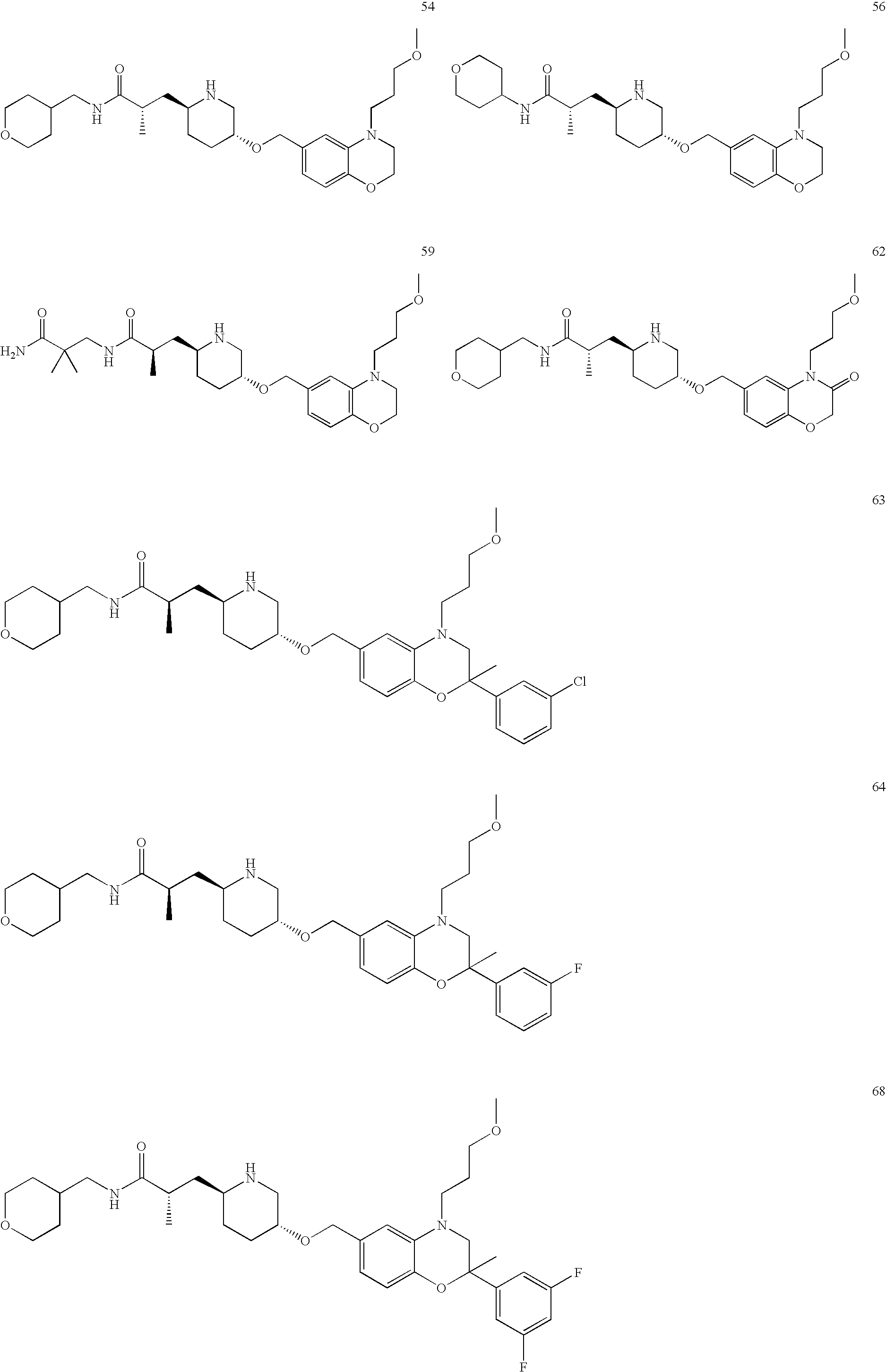

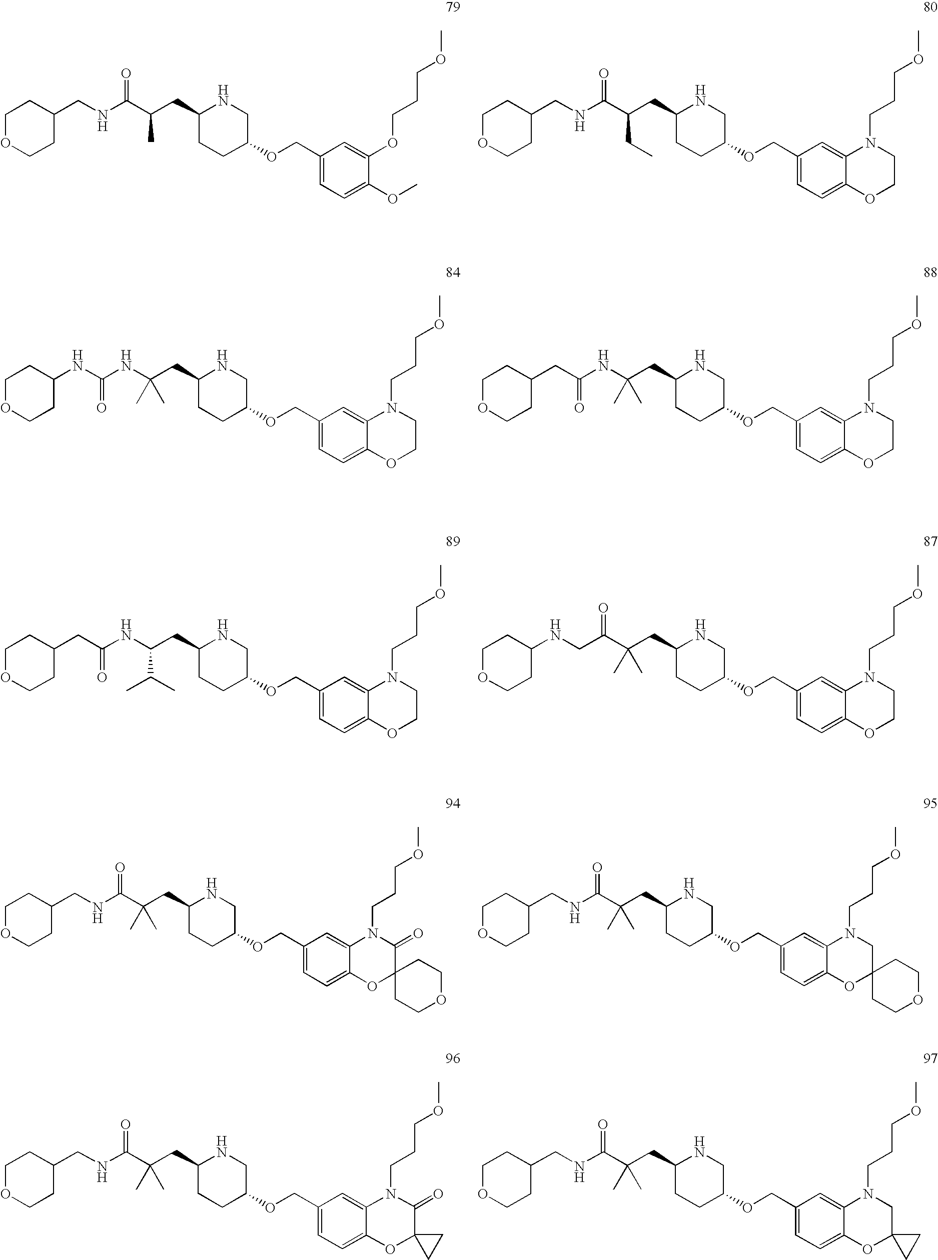

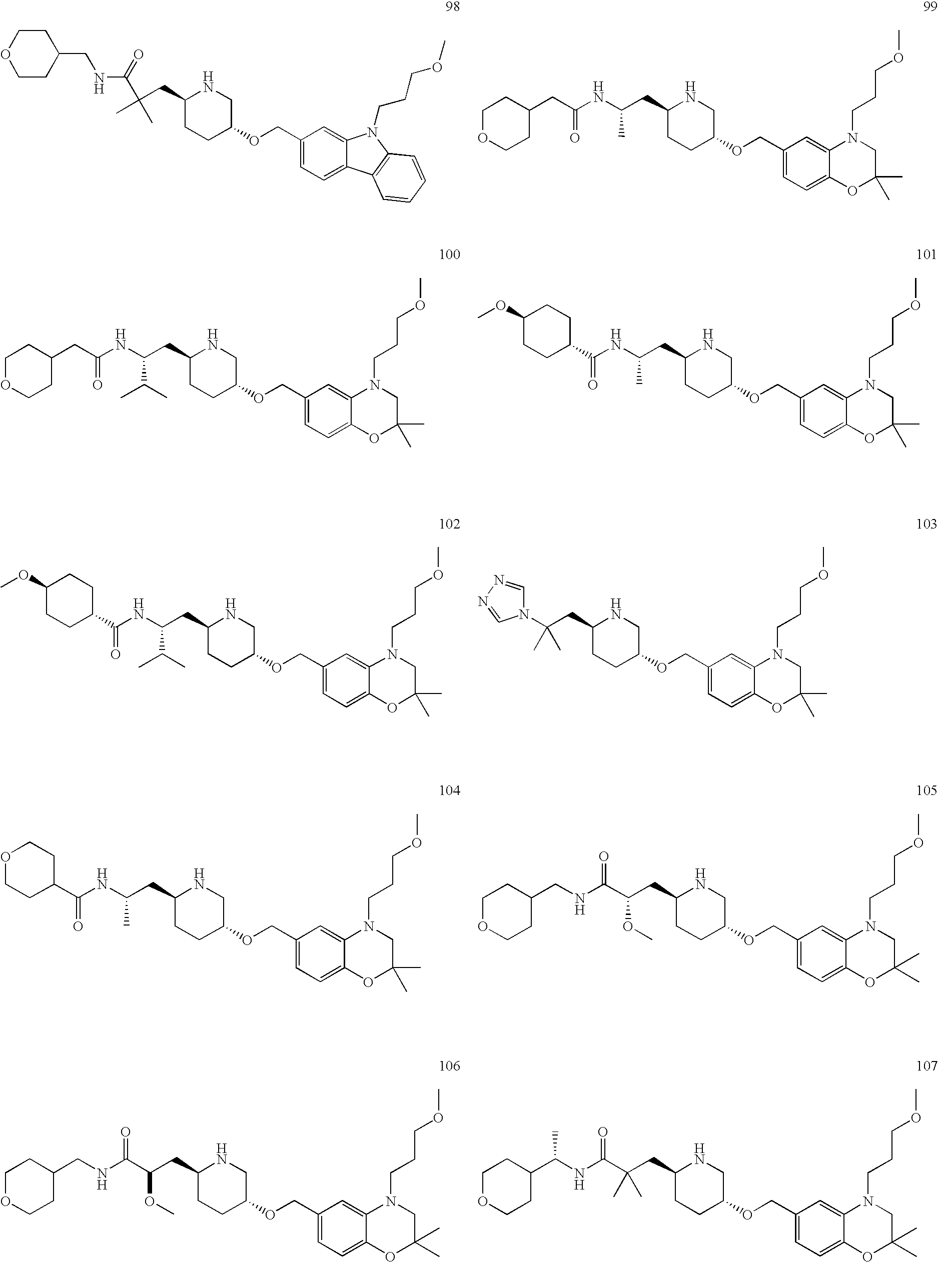

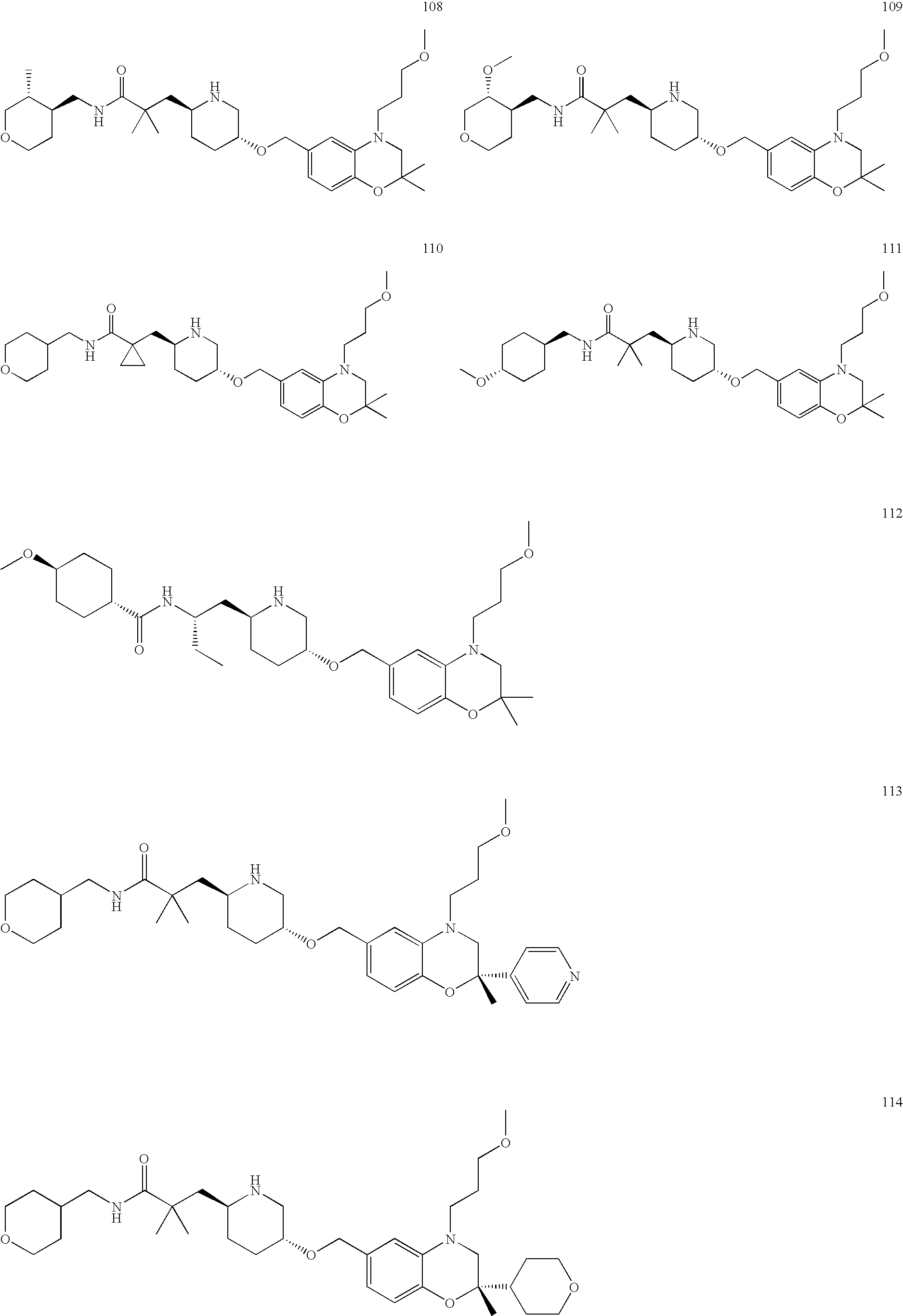

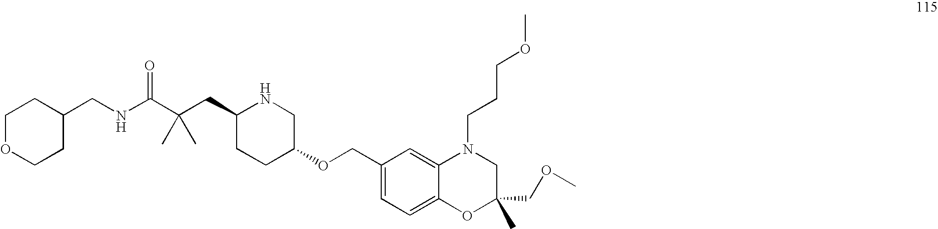

1-12. (canceled)

13. A method for producing a medicament for preventing, for retarding the progression of or for treating hypertension, heart failure, glaucoma, myocardial infarction, kidney failure, restenoses or stroke, which comprises formulating the compound of the general formula (I) 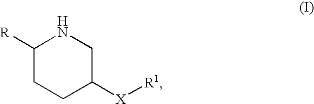
wherein
R is C₂₋₈-alkenyl, C₂₋₈-alkynyl, C₁₋₈-alkyl, C₀₋₈-alkylcarbonyl-(N—C₀₋₈-alkyl)amino-C₁₋₈-alkyl, optionally N-mono- or N,N-di-C₁₋₈-alkylated or -arylated amino-C₁₋₈-alkyl, optionally O—C₁₋₈-alkylated carboxyl-C₀₋₈-alkyl, C₃₋₈-cycloalkyl-C₁₋₈-alkyl, optionally N-mono- or N,N-di-C₃₋₈-cycloalkyl-C₀₋₈-alkylated or optionally N-mono- or N,N-di-heterocyclyl-C₀₋₈-alkylated carbamoyl-C₀₋₈-alkyl, C₃₋₈-cycloalkyl-C₀₋₈-alkylcarbonyl-(N—C₀₋₈-alkyl)amino-C₁₋₈-alkyl, arylcarbonyl-(N—C₀₋₈-alkyl)amino-C₁₋₈-alkyl, optionally N-mono- or N,N-di-C₁₋₈-alkylated or -arylated sulphamoyl-C₀₋₈-alkyl, C₁₋₈-alkylsulphonyl-C₀₋₈-alkyl or heterocyclylcarbonyl-C₀₋₈-alkyl, each of which is either unsubstituted or substituted by 1-4 C₁₋₈-alkoxy, C₁₋₈-alkoxy-C₁₋₈-alkoxy, C₁₋₈-alkoxycarbonyl-(N—C₁₋₈-alkyl)amino, C₁₋₈-alkyl, C₁₋₈-alkylcarbonyl, C₀₋₈-alkylcarbonyl-(N—C₀₋₈-alkyl)amino, C₁₋₈-alkylsulphanyl, C₁₋₈-alkylsulphinyl, C₁₋₈-alkylsulphonyl, aryl-C₀₋₈-alkoxy, aryl, arylamino, aryl-C₀₋₈-alkylsulphonyl, cyano, C₃₋₈-cycloalkoxy, halogen, heterocyclyl, heterocyclyl-C₀₋₈-alkoxy, heterocyclyl-C₀₋₈-alkylamino, heterocyclylcarbonyl, hydroxyl, phosphonyl, optionally N-mono- or N,N-di-C₁₋₈-alkylated amino, optionally N-mono- or N,N-di-C₁₋₈-alkylated carbamoyloxy, optionally N-mono- or N,N-di-C₁₋₈-alkylated sulphamoyl, optionally N-mono-, -di- or -tri-C₁₋₈-alkylated or heterocyclyl-substituted ureido, optionally N-mono- or N,N-di-C₁₋₈-alkylated, N-mono- or N,N-di-arylated or N-mono- or N,N-di-heterocyclyl-C₀₋₈-alkylated amino-C₀₋₈-alkylcarbonyl, oxo or trifluoromethyl;
R¹ is aryl or heterocyclyl, each of which is substituted by 1-4 acyl-C₁₋₈-alkoxy-C₁₋₈-alkoxy, acyl-C₁₋₈-alkoxy-C₁₋₈-alkyl, (N-acyl)-C₁₋₈-alkoxy-C₁₋₈-alkylamino, C₁₋₈-alkanoyl, C₁₋₈-alkoxy, C₁₋₈-alkoxy-C₁₋₈-alkanoyl, C₁₋₈-alkoxy-C₁₋₈-alkoxy, C₁₋₈-alkoxy-C₁₋₈-alkoxy-C₁₋₈-alkyl, C₁₋₈-alkoxy-C₁₋₈-alkyl, (N—C₁₋₈-alkoxy)-C₁₋₈-alkylaminocarbonyl-C₁₋₈-alkoxy, (N—C₁₋₈-alkoxy)-C₁₋₈-alkylaminocarbonyl-C₁₋₈-alkyl, C₁₋₈-alkoxy-C₁₋₈-alkylcarbamoyl, C₁₋₈-alkoxy-C₁₋₈-alkylcarbonyl, C₁₋₈-alkoxy-C₁₋₈-alkylcarbonylamino, 1-C₁₋₈-alkoxy-C₁₋₈-alkylheterocyclyl, C₁₋₈-alkoxyaminocarbonyl-C₁₋₈-alkoxy, C₁₋₈-alkoxyaminocarbonyl-C₁₋₈-alkyl, C₁₋₈-alkoxycarbonyl, C₁₋₈-alkoxycarbonyl-C₁₋₈-alkoxy, C₁₋₈-alkoxycarbonyl-C₁₋₈-alkyl, C₁₋₈-alkoxycarbonylamino-C₁₋₈-alkoxy, C₁₋₈-alkoxycarbonylamino-C₁₋₈-alkyl, C₁₋₈-alkyl, (N—C₁₋₈-alkyl)-C₁₋₈-alkoxy-C₁₋₈-alkylcarbamoyl, (N—C₁₋₈-alkyl)-C₁₋₈-alkoxy-C₁₋₈-alkylcarbonylamino, (N—C₁₋₈-alkyl)-C₁₋₈-alkoxycarbonylamino, (N—C₁₋₈-alkyl)-C₁₋₈-alkylcarbonylamino-C₁₋₈-alkoxy, (N—C₁₋₈-alkyl)-C₁₋₈-alkyl-carbonylamino-C₁₋₈-alkyl, (N—C₁₋₈-alkyl)-C₁₋₈-alkylsulphonylamino-C₁₋₈-alkoxy, (N—C₁₋₈-alkyl)-C₁₋₈-alkylsulphonylamino-C₁₋₈-alkyl, C₁₋₈-alkylamidinyl, C₁₋₈-alkylamino-C₁₋₈-alkoxy, di-C₁₋₈-alkylamino-C₁₋₈-alkoxy, C₁₋₈-alkylamino-C₁₋₈-alkyl, di-C₁₋₈-alkylamino-C₁₋₈-alkyl, C₁₋₈-alkylaminocarbonyl-C₁₋₈-alkoxy, di-C₁₋₈-alkylaminocarbonyl-C₁₋₈-alkoxy, C₁₋₈-alkylaminocarbonyl-C₁₋₈-alkoxy-C₁₋₈-alkyl, C₁₋₈-alkylaminocarbonyl-C₁₋₈-alkyl, di-C₁₋₈-alkylaminocarbonyl-C₁₋₈-alkyl, C₁₋₈-alkylaminocarbonylamino-C₁₋₈-alkoxy, C₁₋₈-alkylaminocarbonylamino-C₁₋₈-alkyl, C₁₋₈-alkylcarbonylamino, C₁₋₈-alkylcarbonylamino-C₁₋₈-alkoxy, C₁₋₈-alkylcarbonylamino-C₁₋₈-alkyl, C₁₋₈-alkylcarbonyloxy-C₁₋₈-alkoxy, C₁₋₈-alkylcarbonyloxy-C₁₋₈-alkyl, C₁₋₈-alkylsulphonyl, C₁₋₈-alkylsulphonyl-C₁₋₈-alkoxy, C₁₋₈-alkylsulphonyl-C₁₋₈-alkyl, C₁₋₈-alkylsulphonylamino-C₁₋₈-alkoxy, C₁₋₈-alkylsulphonylamino-C₁₋₈-alkyl, optionally N-mono- or N,N-di-C₁₋₈-alkylated amino, aryl-C₀₋₈-alkoxy, aryl-C₀₋₈-alkyl, optionally N-mono- or N,N-di-C₁₋₈-alkylated carbamoyl-C₀₋₈-alkoxy, optionally N-mono- or N,N-di-C₁₋₈-alkylated carbamoyl-C₀₋₈-alkyl, carboxy-C₁₋₈-alkoxy, carboxy-C₁₋₈-alkoxy-C₁₋₈-alkyl, carboxy-C₁₋₈-alkyl, cyano, cyano-C₁₋₈-alkoxy, cyano-C₁₋₈-alkyl, C₃₋₈-cycloalkyl-C₁₋₈-alkoxy, C₃₋₈-cycloalkyl-C₁₋₈-alkyl, C₃₋₈-cycloalkylcarbonylamino-C₁₋₈-alkoxy, C₃₋₈-cycloalkylcarbonylamino-C₁₋₈-alkyl, O,N-dimethylhydroxylamino-C₁₋₈-alkyl, halogen, halogen-C₁₋₈-alkoxy, halogen-C₁₋₈-alkyl, halogen-aryl, heterocyclyl-C₀₋₈-alkoxy, heterocyclyl-C₀₋₈-alkyl, heterocyclylcarbonyl, hydroxy-C₁₋₈-alkoxy-C₁₋₈-alkoxy, hydroxy-C₁₋₈-alkoxy-C₁₋₈-alkyl, hydroxy-C₁₋₈-alkyl, O-methyloximyl-C₁₋₈-alkyl, oxide or oxo;
whereby, when R¹ is heterocyclyl and contains at least one saturated carbon atom, this heterocyclyl radical may additionally be substituted at a saturated carbon atom by a C₂₋₈-alkylene chain whose two ends are fixed on this saturated carbon atom and thus form a spirocycle, whereby one CH₂ group of the alkylene chain may be replaced by oxygen;
X is -Alk-, —O-Alk-, -Alk-O—, —O-Alk-O—, —S-Alk-, -Alk-S—, -Alk-NR²—, —NR²-Alk-, —C(O)—NR²—, -Alk-C(O)—NR²—, —C(O)—NR²-Alk-, -Alk-C(O)—NR²-Alk-, —NR²—C(O)—, -Alk-NR²—C(O)—, —NR²—C(O)-Alk-, -Alk-NR²—C(O)-Alk-, —O-Alk-C(O)—NR²—, —O-Alk-NR²—C(O)—, —S(O)₂—NR²—, -Alk-S(O)₂—NR²—, —S(O)₂—NR²-Alk-, -Alk-S(O)₂—NR²-Alk-, —NR²—S(O)₂—, -Alk-NR²—S(O)₂—, —NR²—S(O)₂-Alk- or -Alk-NR²—S(O)₂-Alk-, wherein Alk is C₁₋₈-alkylene which is unsubstituted or substituted by halogen; and wherein
R² is hydrogen, C₁₋₈-alkyl, C₁₋₈-alkoxy-C₁₋₈-alkyl, acyl or aryl-C₁₋₈-alkyl;
or its pharmaceutically usable salt, prodrug or compound, in which one or more atoms have been replaced by their stable, non-radioactive isotopes, as an active ingredient.

14. A method for preventing, for retarding the progression of or for treating hypertension, heart failure, glaucoma, myocardial infarction, kidney failure, restenoses or stroke, which comprises administering, to a subject in need thereof, a therapeutically effective amount of a compound of the general formula (I) 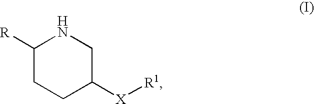
wherein
R is C₂₋₈-alkenyl, C₂₋₈-alkynyl, C₁₋₈-alkyl, C₀₋₈-alkylcarbonyl-(N—C₀₋₈-alkyl)amino-C₁₋₈-alkyl, optionally N-mono- or N,N-di-C₁₋₈-alkylated or -arylated amino-C₁₋₈-alkyl, optionally O—C₁₋₈-alkylated carboxyl-C₀₋₈-alkyl, C₃₋₈-cycloalkyl-C₁₋₈-alkyl, optionally N-mono- or N,N-di-C₃₋₈-cycloalkyl-C₀₋₈-alkylated or optionally N-mono- or N,N-di-heterocyclyl-C₀₋₈-alkylated carbamoyl-C₀₋₈-alkyl, C₃₋₈-cycloalkyl-C₀₋₈-alkylcarbonyl-(N—C₀₋₈-alkyl)amino-C₁₋₈-alkyl, arylcarbonyl-(N—C₀₋₈-alkyl)amino-C₁₋₈-alkyl, optionally N-mono- or N,N-di-C₁₋₈-alkylated or -arylated sulphamoyl-C₀₋₈-alkyl, C₁₋₈-alkylsulphonyl-C₀₋₈-alkyl or heterocyclylcarbonyl-C₀₋₈-alkyl, each of which is either unsubstituted or substituted by 1-4 C₁₋₈-alkoxy, C₁₋₈-alkoxy-C₁₋₈-alkoxy, C₁₋₈-alkoxycarbonyl-(N—C₁₋₈-alkyl)amino, C₁₋₈-alkyl, C₁₋₈-alkylcarbonyl, C₀₋₈-alkylcarbonyl-(N—C₀₋₈-alkyl)amino, C₁₋₈-alkylsulphanyl, C₁₋₈-alkylsulphinyl, C₁₋₈-alkylsulphonyl, aryl-C₀₋₈-alkoxy, aryl, arylamino, aryl-C₀₋₈-alkylsulphonyl, cyano, C₃₋₈-cycloalkoxy, halogen, heterocyclyl, heterocyclyl-C₀₋₈-alkoxy, heterocyclyl-C₀₋₈-alkylamino, heterocyclylcarbonyl, hydroxyl, phosphonyl, optionally N-mono- or N,N-di-C₁₋₈-alkylated amino, optionally N-mono- or N,N-di-C₁₋₈-alkylated carbamoyloxy, optionally N-mono- or N,N-di-C₁₋₈-alkylated sulphamoyl, optionally N-mono-, -di- or -tri-C₁₋₈-alkylated or heterocyclyl-substituted ureido, optionally N-mono- or N,N-di-C₁₋₈-alkylated, N-mono- or N,N-di-arylated or N-mono- or N,N-di-heterocyclyl-C₀₋₈-alkylated amino-C₀₋₈-alkylcarbonyl, oxo or trifluoromethyl;
R¹ is aryl or heterocyclyl, each of which is substituted by 1-4 acyl-C₁₋₈-alkoxy-C₁₋₈-alkoxy, acyl-C₁₋₈-alkoxy-C₁₋₈-alkyl, (N-acyl)-C₁₋₈-alkoxy-C₁₋₈-alkylamino, C₁₋₈-alkanoyl, C₁₋₈-alkoxy, C₁₋₈-alkoxy-C₁₋₈-alkanoyl, C₁₋₈-alkoxy-C₁₋₈-alkoxy, C₁₋₈-alkoxy-C₁₋₈-alkoxy-C₁₋₈-alkyl, C₁₋₈-alkoxy-C₁₋₈-alkyl, (N—C₁₋₈-alkoxy)-C₁₋₈-alkylaminocarbonyl-C₁₋₈-alkoxy, (N—C₁₋₈-alkoxy)-C₁₋₈-alkylaminocarbonyl-C₁₋₈-alkyl, C₁₋₈-alkoxy-C₁₋₈-alkylcarbamoyl, C₁₋₈-alkoxy-C₁₋₈-alkylcarbonyl, C₁₋₈-alkoxy-C₁₋₈-alkylcarbonylamino, 1-C₁₋₈-alkoxy-C₁₋₈-alkylheterocyclyl, C₁₋₈-alkoxyaminocarbonyl-C₁₋₈-alkoxy, C₁₋₈-alkoxyaminocarbonyl-C₁₋₈-alkyl, C₁₋₈-alkoxycarbonyl, C₁₋₈-alkoxycarbonyl-C₁₋₈-alkoxy, C₁₋₈-alkoxycarbonyl-C₁₋₈-alkyl, C₁₋₈-alkoxycarbonylamino-C₁₋₈-alkoxy, C₁₋₈-alkoxycarbonylamino-C₁₋₈-alkyl, C₁₋₈-alkyl, (N—C₁₋₈-alkyl)-C₁₋₈-alkoxy-C₁₋₈-alkylcarbamoyl, (N—C₁₋₈-alkyl)-C₁₋₈-alkoxy-C₁₋₈-alkylcarbonylamino, (N—C₁₋₈-alkyl)-C₁₋₈-alkoxycarbonylamino, (N—C₁₋₈-alkyl)-C₁₋₈-alkylcarbonylamino-C₁₋₈-alkoxy, (N—C₁₋₈-alkyl)-C₁₋₈-alkylcarbonylamino-C₁₋₈-alkyl, (N—C₁₋₈-alkyl)-C₁₋₈-alkylsulphonylamino-C₁₋₈-alkoxy, (N—C₁₋₈-alkyl)-C₁₋₈-alkylsulphonylamino-C₁₋₈-alkyl, C₁₋₈-alkylamidinyl, C₁₋₈-alkylamino-C₁₋₈-alkoxy, di-C₁₋₈-alkylamino-C₁₋₈-alkoxy, C₁₋₈-alkylamino-C₁₋₈-alkyl, di-C₁₋₈-alkylamino-C₁₋₈-alkyl, C₁₋₈-alkylaminocarbonyl-C₁₋₈-alkoxy, di-C₁₋₈-alkylaminocarbonyl-C₁₋₈-alkoxy, C₁₋₈-alkylaminocarbonyl-C₁₋₈-alkoxy-C₁₋₈-alkyl, C₁₋₈-alkylaminocarbonyl-C₁₋₈-alkyl, di-C₁₋₈-alkylaminocarbonyl-C₁₋₈-alkyl, C₁₋₈-alkylaminocarbonylamino-C₁₋₈-alkoxy, C₁₋₈-alkylaminocarbonylamino-C₁₋₈-alkyl, C₁₋₈-alkylcarbonylamino, C₁₋₈-alkylcarbonylamino-C₁₋₈-alkoxy, C₁₋₈-alkylcarbonylamino-C₁₋₈-alkyl, C₁₋₈-alkylcarbonyloxy-C₁₋₈-alkoxy, C₁₋₈-alkylcarbonyloxy-C₁₋₈-alkyl, C₁₋₈-alkylsulphonyl, C₁₋₈-alkylsulphonyl-C₁₋₈-alkoxy, C₁₋₈-alkylsulphonyl-C₁₋₈-alkyl, C₁₋₈-alkylsulphonylamino-C₁₋₈-alkoxy, C₁₋₈-alkylsulphonylamino-C₁₋₈-alkyl, optionally N-mono- or N,N-di-C₁₋₈-alkylated amino, aryl-C₀₋₈-alkoxy, aryl-C₀₋₈-alkyl, optionally N-mono- or N,N-di-C₁₋₈-alkylated carbamoyl-C₀₋₈-alkoxy, optionally N-mono- or N,N-di-C₁₋₈-alkylated carbamoyl-C₀₋₈-alkyl, carboxy-C₁₋₈-alkoxy, carboxy-C₁₋₈-alkoxy-C₁₋₈-alkyl, carboxy-C₁₋₈-alkyl, cyano, cyano-C₁₋₈-alkoxy, cyano-C₁₋₈-alkyl, C₃₋₈-cycloalkyl-C₁₋₈-alkoxy, C₃₋₈-cycloalkyl-C₁₋₈-alkyl, C₃₋₈-cycloalkylcarbonylamino-C₁₋₈-alkoxy, C₃₋₈-cycloalkylcarbonylamino-C₁₋₈-alkyl, O,N-dimethylhydroxylamino-C₁₋₈-alkyl, halogen, halogen-C₁₋₈-alkoxy, halogen-C₁₋₈-alkyl, halogen-aryl, heterocyclyl-C₀₋₈-alkoxy, heterocyclyl-C₀₋₈-alkyl, heterocyclylcarbonyl, hydroxy-C₁₋₈-alkoxy-C₁₋₈-alkoxy, hydroxy-C₁₋₈-alkoxy-C₁₋₈-alkyl, hydroxy-C₁₋₈-alkyl, O-methyloximyl-C₁₋₈-alkyl, oxide or oxo;
whereby, when R¹ is heterocyclyl and contains at least one saturated carbon atom, this heterocyclyl radical may additionally be substituted at a saturated carbon atom by a C₂₋₈-alkylene chain whose two ends are fixed on this saturated carbon atom and thus form a spirocycle, whereby one CH₂ group of the alkylene chain may be replaced by oxygen;
X is -Alk-, —O-Alk-, -Alk-O—, —O-Alk-O—, —S-Alk-, -Alk-S—, -Alk-NR²—, —NR²-Alk-, —C(O)—NR²—, -Alk-C(O)—NR²—, —C(O)—NR²-Alk-, -Alk-C(O)—NR²-Alk-, —NR²—C(O)—, -Alk-NR²—C(O)—, —NR²—C(O)-Alk-, -Alk-NR²—C(O)-Alk-, —O-Alk-C(O)—NR²—, —O-Alk-NR²—C(O)—, —S(O)₂—NR²—, -Alk-S(O)₂—NR²—, —S(O)₂—NR²-Alk-, -Alk-S(O)₂—NR²-Alk-, —NR²—S(O)₂—, -Alk-NR²—S(O)₂—, —NR²—S(O)₂-Alk- or -Alk-NR²—S(O)₂-Alk-, wherein Alk is C₁₋₈-alkylene which is unsubstituted or substituted by halogen; and wherein
R² is hydrogen, C₁₋₈-alkyl, C₁₋₈-alkoxy-C₁₋₈-alkyl, acyl or aryl-C₁₋₈-alkyl;
or its pharmaceutically usable salt.

15. A method according to claim 13, wherein the compound of general formula (I) is a compound of the general formula (IA) 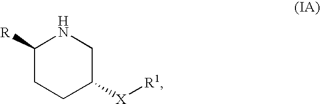
or a pharmaceutically usable salt thereof, in which R, R¹ and X are each as defined for the compound of the formula (I) according to claim 13.

16. A pharmaceutical composition having renin-inhibiting properties and comprising a compound of the general formula (I) 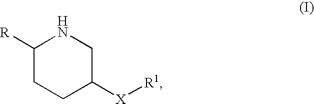
wherein
R is C₂₋₈-alkenyl, C₂₋₈-alkynyl, C₁₋₈-alkyl, C₀₋₈-alkylcarbonyl-(N—C₀₋₈-alkyl)amino-C₁₋₈-alkyl, optionally N-mono- or N,N-di-C₁₋₈-alkylated or -arylated amino-C₁₋₈-alkyl, optionally O—C₁₋₈-alkylated carboxyl-C₀₋₈-alkyl, C₃₋₈-cycloalkyl-C₁₋₈-alkyl, optionally N-mono- or N,N-di-C₃₋₈-cycloalkyl-C₀₋₈-alkylated or optionally N-mono- or N,N-di-heterocyclyl-C₀₋₈-alkylated carbamoyl-C₀₋₈-alkyl, C₃₋₈-cycloalkyl-C₀₋₈-alkylcarbonyl-(N—C₀₋₈-alkyl)amino-C₁₋₈-alkyl, arylcarbonyl-(N—C₀₋₈-alkyl)amino-C₁₋₈-alkyl, optionally N-mono- or N,N-di-C₁₋₈-alkylated or -arylated sulphamoyl-C₀₋₈-alkyl, C₁₋₈-alkylsulphonyl-C₀₋₈-alkyl or heterocyclylcarbonyl-C₀₋₈-alkyl, each of which is either unsubstituted or substituted by 1-4 C₁₋₈-alkoxy, C₁₋₈-alkoxy-C₁₋₈-alkoxy, C₁₋₈-alkoxycarbonyl-(N—C₁₋₈-alkyl)amino, C₁₋₈-alkyl, C₁₋₈-alkylcarbonyl, C₀₋₈-alkylcarbonyl-(N—C₀₋₈-alkyl)amino, C₁₋₈-alkylsulphanyl, C₁₋₈-alkylsulphinyl, C₁₋₈-alkylsulphonyl, aryl-C₀₋₈-alkoxy, aryl, arylamino, aryl-C₀₋₈-alkylsulphonyl, cyano, C₃₋₈-cycloalkoxy, halogen, heterocyclyl, heterocyclyl-C₀₋₈-alkoxy, heterocyclyl-C₀₋₈-alkylamino, heterocyclylcarbonyl, hydroxyl, phosphonyl, optionally N-mono- or N,N-di-C₁₋₈-alkylated amino, optionally N-mono- or N,N-di-C₁₋₈-alkylated carbamoyloxy, optionally N-mono- or N,N-di-C₁₋₈-alkylated sulphamoyl, optionally N-mono-, -di- or -tri-C₁₋₈-alkylated or heterocyclyl-substituted ureido, optionally N-mono- or N,N-di-C₁₋₈-alkylated, N-mono- or N,N-di-arylated or N-mono- or N,N-di-heterocyclyl-C₀₋₈-alkylated amino-C₀₋₈-alkylcarbonyl, oxo or trifluoromethyl;
R¹ is aryl or heterocyclyl, each of which is substituted by 1-4 acyl-C₁₋₈-alkoxy-C₁₋₈-alkoxy, acyl-C₁₋₈-alkoxy-C₁₋₈-alkyl, (N-acyl)-C₁₋₈-alkoxy-C₁₋₈-alkylamino, C₁₋₈-alkanoyl, C₁₋₈-alkoxy, C₁₋₈-alkoxy-C₁₋₈-alkanoyl, C₁₋₈-alkoxy-C₁₋₈-alkoxy, C₁₋₈-alkoxy-C₁₋₈-alkoxy-C₁₋₈-alkyl, C₁₋₈-alkoxy-C₁₋₈-alkyl, (N—C₁₋₈-alkoxy)-C₁₋₈-alkylaminocarbonyl-C₁₋₈-alkoxy, (N—C₁₋₈-alkoxy)-C₁₋₈-alkylaminocarbonyl-C₁₋₈-alkyl, C₁₋₈-alkoxy-C₁₋₈-alkylcarbamoyl, C₁₋₈-alkoxy-C₁₋₈-alkylcarbonyl, C₁₋₈-alkoxy-C₁₋₈-alkylcarbonylamino, 1-C₁₋₈-alkoxy-C₁₋₈-alkylheterocyclyl, C₁₋₈-alkoxyaminocarbonyl-C₁₋₈-alkoxy, C₁₋₈-alkoxyaminocarbonyl-C₁₋₈-alkyl, C₁₋₈-alkoxycarbonyl, C₁₋₈-alkoxycarbonyl-C₁₋₈-alkoxy, C₁₋₈-alkoxycarbonyl-C₁₋₈-alkyl, C₁₋₈-alkoxycarbonylamino-C₁₋₈-alkoxy, C₁₋₈-alkoxycarbonylamino-C₁₋₈-alkyl, C₁₋₈-alkyl, (N—C₁₋₈-alkyl)-C₁₋₈-alkoxy-C₁₋₈-alkylcarbamoyl, (N—C₁₋₈-alkyl)-C₁₋₈-alkoxy-C₁₋₈-alkylcarbonylamino, (N—C₁₋₈-alkyl)-C₁₋₈-alkoxycarbonylamino, (N—C₁₋₈-alkyl)-C₁₋₈-alkylcarbonylamino-C₁₋₈-alkoxy, (N—C₁₋₈-alkyl)-C₁₋₈-alkylcarbonylamino-C₁₋₈-alkyl, (N—C₁₋₈-alkyl)-C₁₋₈-alkylsulphonylamino-C₁₋₈-alkoxy, (N—C₁₋₈-alkyl)-C₁₋₈-alkylsulphonylamino-C₁₋₈-alkyl, C₁₋₈-alkylamidinyl, C₁₋₈-alkylamino-C₁₋₈-alkoxy, di-C₁₋₈-alkylamino-C₁₋₈-alkoxy, C₁₋₈-alkylamino-C₁₋₈-alkyl, di-C₁₋₈-alkylamino-C₁₋₈-alkyl, C₁₋₈-alkylaminocarbonyl-C₁₋₈-alkoxy, di-C₁₋₈-alkylaminocarbonyl-C₁₋₈-alkoxy, C₁₋₈-alkylaminocarbonyl-C₁₋₈-alkoxy-C₁₋₈-alkyl, C₁₋₈-alkylaminocarbonyl-C₁₋₈-alkyl, di-C₁₋₈-alkylaminocarbonyl-C₁₋₈-alkyl, C₁₋₈-alkylaminocarbonylamino-C₁₋₈-alkoxy, C₁₋₈-alkylaminocarbonylamino-C₁₋₈-alkyl, C₁₋₈-alkylcarbonylamino, C₁₋₈-alkylcarbonylamino-C₁₋₈-alkoxy, C₁₋₈-alkylcarbonylamino-C₁₋₈-alkyl, C₁₋₈-alkylcarbonyloxy-C₁₋₈-alkoxy, C₁₋₈-alkylcarbonyloxy-C₁₋₈-alkyl, C₁₋₈-alkylsulphonyl, C₁₋₈-alkylsulphonyl-C₁₋₈-alkoxy, C₁₋₈-alkylsulphonyl-C₁₋₈-alkyl, C₁₋₈-alkylsulphonylamino-C₁₋₈-alkoxy, C₁₋₈-alkylsulphonylamino-C₁₋₈-alkyl, optionally N-mono- or N,N-di-C₁₋₈-alkylated amino, aryl-C₀₋₈-alkoxy, aryl-C₀₋₈-alkyl, optionally N-mono- or N,N-di-C₁₋₈-alkylated carbamoyl-C₀₋₈-alkoxy, optionally N-mono- or N,N-di-C₁₋₈-alkylated carbamoyl-C₀₋₈-alkyl, carboxy-C₁₋₈-alkoxy, carboxy-C₁₋₈-alkoxy-C₁₋₈-alkyl, carboxy-C₁₋₈-alkyl, cyano, cyano-C₁₋₈-alkoxy, cyano-C₁₋₈-alkyl, C₃₋₈-cycloalkyl-C₁₋₈-alkoxy, C₃₋₈-cycloalkyl-C₁₋₈-alkyl, C₃₋₈-cycloalkylcarbonylamino-C₁₋₈-alkoxy, C₃₋₈-cycloalkylcarbonylamino-C₁₋₈-alkyl, O,N-dimethylhydroxylamino-C₁₋₈-alkyl, halogen, halogen-C₁₋₈-alkoxy, halogen-C₁₋₈-alkyl, halogen-aryl, heterocyclyl-C₀₋₈-alkoxy, heterocyclyl-C₀₋₈-alkyl, heterocyclylcarbonyl, hydroxy-C₁₋₈-alkoxy-C₁₋₈-alkoxy, hydroxy-C₁₋₈-alkoxy-C₁₋₈-alkyl, hydroxy-C₁₋₈-alkyl, O-methyloximyl-C₁₋₈-alkyl, oxide or oxo;
whereby, when R¹ is heterocyclyl and contains at least one saturated carbon atom, this heterocyclyl radical may additionally be substituted at a saturated carbon atom by a C₂₋₈-alkylene chain whose two ends are fixed on this saturated carbon atom and thus form a spirocycle, whereby one CH₂ group of the alkylene chain may be replaced by oxygen;
X is -Alk-, —O-Alk-, -Alk-O—, —O-Alk-O—, —S-Alk-, -Alk-S—, -Alk-NR²—, —NR²-Alk-, —C(O)—NR²—, -Alk-C(O)—NR²—, —C(O)—NR²-Alk-, -Alk-C(O)—NR²-Alk-, —NR²—C(O)—, -Alk-NR²—C(O)—, —NR²—C(O)-Alk-, -Alk-NR²—C(O)-Alk-, —O-Alk-C(O)—NR²—, —O-Alk-NR²—C(O)—, —S(O)₂—NR²—, -Alk-S(O)₂—NR²—, —S(O)₂—NR²-Alk-, -Alk-S(O)₂—NR²-Alk-, —NR²—S(O)₂—, -Alk-NR²—S(O)₂—, —NR²—S(O)₂-Alk- or -Alk-NR²—S(O)₂-Alk-, wherein Alk is C₁₋₈-alkylene which is unsubstituted or substituted by halogen; and wherein
R² is hydrogen, C₁₋₈-alkyl, C₁₋₈-alkoxy-C₁₋₈-alkyl, acyl or aryl-C₁₋₈-alkyl;
or its pharmaceutically usable salt, and customary excipients.

17. A pharmaceutical composition having renin-inhibiting properties, in the form of a preparation or of a kit composed of individual components, consisting of a) a compound of the general formula (I) 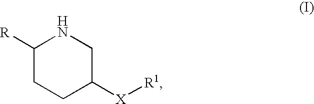
wherein
R is C₂₋₈-alkenyl, C₂₋₈-alkynyl, C₁₋₈-alkyl, C₀₋₈-alkylcarbonyl-(N—C₀₋₈-alkyl)amino-C₁₋₈-alkyl, optionally N-mono- or N,N-di-C₁₋₈-alkylated or -arylated amino-C₁₋₈-alkyl, optionally O—C₁₋₈-alkylated carboxyl-C₀₋₈-alkyl, C₃₋₈-cycloalkyl-C₁₋₈-alkyl, optionally N-mono- or N,N-di-C₃₋₈-cycloalkyl-C₀₋₈-alkylated or optionally N-mono- or N,N-di-heterocyclyl-C₀₋₈-alkylated carbamoyl-C₀₋₈-alkyl, C₃₋₈-cycloalkyl-C₀₋₈-alkylcarbonyl-(N—C₀₋₈-alkyl)amino-C₁₋₈-alkyl, arylcarbonyl-(N—C₀₋₈-alkyl)amino-C₁₋₈-alkyl, optionally N-mono- or N,N-di-C₁₋₈-alkylated or -arylated sulphamoyl-C₀₋₈-alkyl, C₁₋₈-alkylsulphonyl-C₀₋₈-alkyl or heterocyclylcarbonyl-C₀₋₈-alkyl, each of which is either unsubstituted or substituted by 1-4 C₁₋₈-alkoxy, C₁₋₈-alkoxy-C₁₋₈-alkoxy, C₁₋₈-alkoxycarbonyl-(N—C₁₋₈-alkyl)amino, C₁₋₈-alkyl, C₁₋₈-alkylcarbonyl, C₀₋₈-alkylcarbonyl-(N—C₀₋₈-alkyl)amino, C₁₋₈-alkylsulphanyl, C₁₋₈-alkylsulphinyl, C₁₋₈-alkylsulphonyl, aryl-C₀₋₈-alkoxy, aryl, arylamino, aryl-C₀₋₈-alkylsulphonyl, cyano, C₃₋₈-cycloalkoxy, halogen, heterocyclyl, heterocyclyl-C₀₋₈-alkoxy, heterocyclyl-C₀₋₈-alkylamino, heterocyclylcarbonyl, hydroxyl, phosphonyl, optionally N-mono- or N,N-di-C₁₋₈-alkylated amino, optionally N-mono- or N,N-di-C₁₋₈-alkylated carbamoyloxy, optionally N-mono- or N,N-di-C₁₋₈-alkylated sulphamoyl, optionally N-mono-, -di- or -tri-C₁₋₈-alkylated or heterocyclyl-substituted ureido, optionally N-mono- or N,N-di-C₁₋₈-alkylated, N-mono- or N,N-di-arylated or N-mono- or N,N-di-heterocyclyl-C₀₋₈-alkylated amino-C₀₋₈-alkylcarbonyl, oxo or trifluoromethyl;
R¹ is aryl or heterocyclyl, each of which is substituted by 1-4 acyl-C₁₋₈-alkoxy-C₁₋₈-alkoxy, acyl-C₁₋₈-alkoxy-C₁₋₈-alkyl, (N-acyl)-C₁₋₈-alkoxy-C₁₋₈-alkylamino, C₁₋₈-alkanoyl, C₁₋₈-alkoxy, C₁₋₈-alkoxy-C₁₋₈-alkanoyl, C₁₋₈-alkoxy-C₁₋₈-alkoxy, C₁₋₈-alkoxy-C₁₋₈-alkoxy-C₁₋₈-alkyl, C₁₋₈-alkoxy-C₁₋₈-alkyl, (N—C₁₋₈-alkoxy)-C₁₋₈-alkylaminocarbonyl-C₁₋₈-alkoxy, (N—C₁₋₈-alkoxy)-C₁₋₈-alkylaminocarbonyl-C₁₋₈-alkyl, C₁₋₈-alkoxy-C₁₋₈-alkylcarbamoyl, C₁₋₈-alkoxy-C₁₋₈-alkylcarbonyl, C₁₋₈-alkoxy-C₁₋₈-alkylcarbonylamino, 1-C₁₋₈-alkoxy-C₁₋₈-alkylheterocyclyl, C₁₋₈-alkoxyaminocarbonyl-C₁₋₈-alkoxy, C₁₋₈-alkoxyaminocarbonyl-C₁₋₈-alkyl, C₁₋₈-alkoxycarbonyl, C₁₋₈-alkoxycarbonyl-C₁₋₈-alkoxy, C₁₋₈-alkoxycarbonyl-C₁₋₈-alkyl, C₁₋₈-alkoxycarbonylamino-C₁₋₈-alkoxy, C₁₋₈-alkoxycarbonylamino-C₁₋₈-alkyl, C₁₋₈-alkyl, (N—C₁₋₈-alkyl)-C₁₋₈-alkoxy-C₁₋₈-alkylcarbamoyl, (N—C₁₋₈-alkyl)-C₁₋₈-alkoxy-C₁₋₈-alkylcarbonylamino, (N—C₁₋₈-alkyl)-C₁₋₈-alkoxycarbonylamino, (N—C₁₋₈-alkyl)-C₁₋₈-alkylcarbonylamino-C₁₋₈-alkoxy, (N—C₁₋₈-alkyl)-C₁₋₈-alkylcarbonylamino-C₁₋₈-alkyl, (N—C₁₋₈-alkyl)-C₁₋₈-alkylsulphonylamino-C₁₋₈-alkoxy, (N—C₁₋₈-alkyl)-C₁₋₈-alkylsulphonylamino-C₁₋₈-alkyl, C₁₋₈-alkylamidinyl, C₁₋₈-alkylamino-C₁₋₈-alkoxy, di-C₁₋₈-alkylamino-C₁₋₈-alkoxy, C₁₋₈-alkylamino-C₁₋₈-alkyl, di-C₁₋₈-alkylamino-C₁₋₈-alkyl, C₁₋₈-alkylaminocarbonyl-C₁₋₈-alkoxy, di-C₁₋₈-alkylaminocarbonyl-C₁₋₈-alkoxy, C₁₋₈-alkylaminocarbonyl-C₁₋₈-alkoxy-C₁₋₈-alkyl, C₁₋₈-alkylaminocarbonyl-C₁₋₈-alkyl, di-C₁₋₈-alkylaminocarbonyl-C₁₋₈-alkyl, C₁₋₈-alkylaminocarbonylamino-C₁₋₈-alkoxy, C₁₋₈-alkylaminocarbonylamino-C₁₋₈-alkyl, C₁₋₈-alkylcarbonylamino, C₁₋₈-alkylcarbonylamino-C₁₋₈-alkoxy, C₁₋₈-alkylcarbonylamino-C₁₋₈-alkyl, C₁₋₈-alkylcarbonyloxy-C₁₋₈-alkoxy, C₁₋₈-alkylcarbonyloxy-C₁₋₈-alkyl, C₁₋₈-alkylsulphonyl, C₁₋₈-alkylsulphonyl-C₁₋₈-alkoxy, C₁₋₈-alkylsulphonyl-C₁₋₈-alkyl, C₁₋₈-alkylsulphonylamino-C₁₋₈-alkoxy, C₁₋₈-alkylsulphonylamino-C₁₋₈-alkyl, optionally N-mono- or N,N-di-C₁₋₈-alkylated amino, aryl-C₀₋₈-alkoxy, aryl-C₀₋₈-alkyl, optionally N-mono- or N,N-di-C₁₋₈-alkylated carbamoyl-C₀₋₈-alkoxy, optionally N-mono- or N,N-di-C₁₋₈-alkylated carbamoyl-C₀₋₈-alkyl, carboxy-C₁₋₈-alkoxy, carboxy-C₁₋₈-alkoxy-C₁₋₈-alkyl, carboxy-C₁₋₈-alkyl, cyano, cyano-C₁₋₈-alkoxy, cyano-C₁₋₈-alkyl, C₃₋₈-cycloalkyl-C₁₋₈-alkoxy, C₃₋₈-cycloalkyl-C₁₋₈-alkyl, C₃₋₈-cycloalkylcarbonylamino-C₁₋₈-alkoxy, C₃₋₈-cycloalkylcarbonylamino-C₁₋₈-alkyl, O,N-dimethylhydroxylamino-C₁₋₈-alkyl, halogen, halogen-C₁₋₈-alkoxy, halogen-C₁₋₈-alkyl, halogen-aryl, heterocyclyl-C₀₋₈-alkoxy, heterocyclyl-C₀₋₈-alkyl, heterocyclylcarbonyl, hydroxy-C₁₋₈-alkoxy-C₁₋₈-alkoxy, hydroxy-C₁₋₈-alkoxy-C₁₋₈-alkyl, hydroxy-C₁₋₈-alkyl, O-methyloximyl-C₁₋₈-alkyl, oxide or oxo;
whereby, when R¹ is heterocyclyl and contains at least one saturated carbon atom, this heterocyclyl radical may additionally be substituted at a saturated carbon atom by a C₂₋₈-alkylene chain whose two ends are fixed on this saturated carbon atom and thus form a spirocycle, whereby one CH₂ group of the alkylene chain may be replaced by oxygen;
X is -Alk-, —O-Alk-, -Alk-O—, —O-Alk-O—, —S-Alk-, -Alk-S—, -Alk-NR²—, —NR²-Alk-, —C(O)—NR²—, -Alk-C(O)—NR²—, —C(O)—NR²-Alk-, -Alk-C(O)—NR²-Alk-, —NR²—C(O)—, -Alk-NR²—C(O)—, —NR²—C(O)-Alk-, -Alk-NR²—C(O)-Alk-, —O-Alk-C(O)—NR²—, —O-Alk-NR²—C(O)—, —S(O)₂—NR²—, -Alk-S(O)₂—NR²—, —S(O)₂—NR²-Alk-, -Alk-S(O)₂—NR²-Alk-, —NR²—S(O)₂—, -Alk-NR²—S(O)₂—, —NR²—S(O)₂-Alk- or -Alk-NR²—S(O)₂-Alk-, wherein Alk is C₁₋₈-alkylene which is unsubstituted or substituted by halogen; and wherein
R² is hydrogen, C₁₋₈-alkyl, C₁₋₈-alkoxy-C₁₋₈-alkyl, acyl or aryl-C₁₋₈-alkyl;
or its pharmaceutically usable salt,
and b) at least one agent having cardiovascular action.

18. A composition according to claim 16, wherein the compound of general formula (I) is a compound of the general formula (IA) 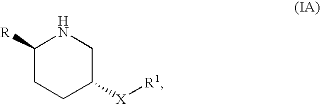
or a pharmaceutically usable salt thereof, in which R, R¹ and X are each as defined for the compound of the formula (I) according to claim 16.

19. A compound of the general formula (I) 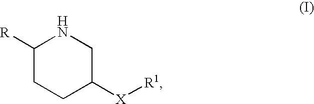
or a salt, especially a pharmaceutically usable salt, thereof, wherein
R is C₂₋₈-alkenyl, C₂₋₈-alkynyl, C₁₋₈-alkyl, C₀₋₈-alkylcarbonyl-(N—C₀₋₈-alkyl)amino-C₁₋₈-alkyl, optionally N-mono- or N,N-di-C₁₋₈-alkylated or -arylated amino-C₁₋₈-alkyl, optionally O—C₁₋₈-alkylated carboxyl-C₀₋₈-alkyl, C₃₋₈-cycloalkyl-C₁₋₈-alkyl, optionally N-mono- or N,N-di-C₃₋₈-cycloalkyl-C₀₋₈-alkylated or optionally N-mono- or N,N-di-heterocyclyl-C₀₋₈-alkylated carbamoyl-C₀₋₈-alkyl, C₃₋₈-cycloalkyl-C₀₋₈-alkylcarbonyl-(N—C₀₋₈-alkyl)amino-C₁₋₈-alkyl, arylcarbonyl-(N—C₀₋₈-alkyl)amino-C₁₋₈-alkyl, optionally N-mono- or N,N-di-C₁₋₈-alkylated or -arylated sulphamoyl-C₀₋₈-alkyl, C₁₋₈-alkylsulphonyl-C₀₋₈-alkyl or heterocyclylcarbonyl-C₀₋₈-alkyl, each of which is either unsubstituted or substituted by 1-4 C₁₋₈-alkoxy, C₁₋₈-alkoxy-C₁₋₈-alkoxy, C₁₋₈-alkoxycarbonyl-(N—C₁₋₈-alkyl)amino, C₁₋₈-alkyl, C₁₋₈-alkylcarbonyl, C₀₋₈-alkylcarbonyl-(N—C₀₋₈-alkyl)amino, C₁₋₈-alkylsulphanyl, C₁₋₈-alkylsulphinyl, C₁₋₈-alkylsulphonyl, aryl-C₀₋₈-alkoxy, aryl, arylamino, aryl-C₀₋₈-alkylsulphonyl, cyano, C₃₋₈-cycloalkoxy, halogen, heterocyclyl, heterocyclyl-C₀₋₈-alkoxy, heterocyclyl-C₀₋₈-alkylamino, heterocyclylcarbonyl, hydroxyl, phosphonyl, optionally N-mono- or N,N-di-C₁₋₈-alkylated amino, optionally N-mono- or N,N-di-C₁₋₈-alkylated carbamoyloxy, optionally N-mono- or N,N-di-C₁₋₈-alkylated sulphamoyl, optionally N-mono-, -di- or -tri-C₁₋₈-alkylated or heterocyclyl-substituted ureido, optionally N-mono- or N,N-di-C₁₋₈-alkylated, N-mono- or N,N-di-arylated or N-mono- or N,N-di-heterocyclyl-C₀₋₈-alkylated amino-C₀₋₈-alkylcarbonyl, oxo or trifluoromethyl;
R¹ is aryl or heterocyclyl, each of which is substituted by 1-4 acyl-C₁₋₈-alkoxy-C₁₋₈-alkoxy, acyl-C₁₋₈-alkoxy-C₁₋₈-alkyl, (N-acyl)-C₁₋₈-alkoxy-C₁₋₈-alkylamino, C₁₋₈-alkanoyl, C₁₋₈-alkoxy, C₁₋₈-alkoxy-C₁₋₈-alkanoyl, C₁₋₈-alkoxy-C₁₋₈-alkoxy, C₁₋₈-alkoxy-C₁₋₈-alkoxy-C₁₋₈-alkyl, C₁₋₈-alkoxy-C₁₋₈-alkyl, (N—C₁₋₈-alkoxy)-C₁₋₈-alkylaminocarbonyl-C₁₋₈-alkoxy, (N—C₁₋₈-alkoxy)-C₁₋₈-alkylaminocarbonyl-C₁₋₈-alkyl, C₁₋₈-alkoxy-C₁₋₈-alkylcarbamoyl, C₁₋₈-alkoxy-C₁₋₈-alkylcarbonyl, C₁₋₈-alkoxy-C₁₋ₛ-alkylcarbonylamino, 1-C₁₋₈-alkoxy-C₁₋₈-alkylheterocyclyl, C₁₋₈-alkoxyaminocarbonyl-C₁₋₈-alkoxy, C₁₋₈-alkoxyaminocarbonyl-C₁₋₈-alkyl, C₁₋₈-alkoxycarbonyl, C₁₋₈-alkoxycarbonyl-C₁₋₈-alkoxy, C₁₋₈-alkoxycarbonyl-C₁₋₈-alkyl, C₁₋₈-alkoxycarbonylamino-C₁₋₈-alkoxy, C₁₋₈-alkoxycarbonylamino-C₁₋₈-alkyl, C₁₋₈-alkyl, (N—C₁₋₈-alkyl)-C₁₋₈-alkoxy-C₁₋₈-alkylcarbamoyl, (N—C₁₋₈-alkyl)-C₁₋₈-alkoxy-C₁₋₈-alkylcarbonylamino, (N—C₁₋₈-alkyl)-C₁₋₈-alkoxycarbonylamino, (N—C₁₋₈-alkyl)-C₁₋₈-alkylcarbonylamino-C₁₋₈-alkoxy, (N—C₁₋₈-alkyl)-C₁₋₈-alkylcarbonylamino-C₁₋₈-alkyl, (N—C₁₋₈-alkyl)-C₁₋₈-alkylsulphonylamino-C₁₋₈-alkoxy, (N—C₁₋₈-alkyl)-C₁₋₈-alkylsulphonylamino-C₁₋₈-alkyl, C₁₋₈-alkylamidinyl, C₁₋₈-alkylamino-C₁₋₈-alkoxy, di-C₁₋₈-alkylamino-C₁₋₈-alkoxy, C₁₋₈-alkylamino-C₁₋₈-alkyl, di-C₁₋₈-alkylamino-C₁₋₈-alkyl, C₁₋₈-alkylaminocarbonyl-C₁₋₈-alkoxy, di-C₁₋₈-alkylaminocarbonyl-C₁₋₈-alkoxy, C₁₋₈-alkylaminocarbonyl-C₁₋₈-alkoxy-C₁₋₈-alkyl, C₁₋₈-alkylaminocarbonyl-C₁₋₈-alkyl, di-C₁₋₈-alkylaminocarbonyl-C₁₋₈-alkyl, C₁₋₈-alkylaminocarbonylamino-C₁₋₈-alkoxy, C₁₋₈-alkylaminocarbonylamino-C₁₋₈-alkyl, C₁₋₈-alkylcarbonylamino, C₁₋₈-alkylcarbonylamino-C₁₋₈-alkoxy, C₁₋₈-alkylcarbonylamino-C₁₋₈-alkyl, C₁₋₈-alkylcarbonyloxy-C₁₋₈-alkoxy, C₁₋₈-alkylcarbonyloxy-C₁₋₈-alkyl, C₁₋₈-alkylsulphonyl, C₁₋₈-alkylsulphonyl-C₁₋₈-alkoxy, C₁₋₈-alkylsulphonyl-C₁₋₈-alkyl, C₁₋₈-alkylsulphonylamino-C₁₋₈-alkoxy, C₁₋₈-alkylsulphonylamino-C₁₋₈-alkyl, optionally N-mono- or N,N-di-C₁₋₈-alkylated amino, aryl-C₀₋₈-alkoxy, aryl-C₀₋₈-alkyl, optionally N-mono- or N,N-di-C₁₋₈-alkylated carbamoyl-C₀₋₈-alkoxy, optionally N-mono- or N,N-di-C₁₋₈-alkylated carbamoyl-C₀₋₈-alkyl, carboxy-C₁₋₈-alkoxy, carboxy-C₁₋₈-alkoxy-C₁₋₈-alkyl, carboxy-C₁₋₈-alkyl, cyano, cyano-C₁₋₈-alkoxy, cyano-C₁₋₈-alkyl, C₃₋₈-cycloalkyl-C₁₋₈-alkoxy, C₃₋₈-cycloalkyl-C₁₋₈-alkyl, C₃₋₈-cycloalkylcarbonylamino-C₁₋₈-alkoxy, C₃₋₈-cycloalkylcarbonylamino-C₁₋₈-alkyl, O,N-dimethylhydroxylamino-C₁₋₈-alkyl, halogen, halogen-C₁₋₈-alkoxy, halogen-C₁₋₈-alkyl, halogen-aryl, heterocyclyl-C₀₋₈-alkoxy, heterocyclyl-C₀₋₈-alkyl, heterocyclylcarbonyl, hydroxy-C₁₋₈-alkoxy-C₁₋₈-alkoxy, hydroxy-C₁₋₈-alkoxy-C₁₋₈-alkyl, hydroxy-C₁₋₈-alkyl, O-methyloximyl-C₁₋₈-alkyl, oxide or oxo;
whereby, when R¹ is heterocyclyl and contains at least one saturated carbon atom, this heterocyclyl radical may additionally be substituted at a saturated carbon atom by a C₂₋₈-alkylene chain whose two ends are fixed on this saturated carbon atom and thus form a spirocycle, whereby one CH₂ group of the alkylene chain may be replaced by oxygen;
X is Alk-, —O-Alk-, -Alk-O—, —O-Alk-O—, —S-Alk-, -Alk-S—, -Alk-NR²—, —NR²-Alk-, —C(O)—NR²—, -Alk-C(O)—NR²—, —C(O)—NR²-Alk-, -Alk-C(O)—NR²-Alk-, —NR²—C(O)—, -Alk-NR²—C(O)—, —NR²—C(O)-Alk-, -Alk-NR²—C(O)-Alk-, —O-Alk-C(O)—NR²—, —O-Alk-NR²—C(O)—, —S(O)₂—NR²—, -Alk-S(O)₂—NR²—, —S(O)₂—NR²-Alk-, -Alk-S(O)₂—NR²-Alk-, —NR²—S(O)₂—, -Alk-NR²—S(O)₂—, —NR²—S(O)₂-Alk- or -Alk-NR²—S(O)₂-Alk-, wherein Alk is C₁₋₈-alkylene, which is unsubstituted or substituted by halogen; where
R² is hydrogen, C₁₋₈-alkyl, C₁₋₈-alkoxy-C₁₋₈-alkyl, acyl or aryl-C₁₋₈-alkyl;
and wherein,
when X is —C(O)—NR²—, -Alk-C(O)—NR²—, —C(O)—NR²-Alk-, -Alk-C(O)—NR²-Alk-, —NR²—C(O)—, -Alk-NR²—C(O)—, —NR²—C(O)-Alk-, -Alk-NR²—C(O)-Alk-, —O-Alk-C(O)—NR²—, —O-Alk-NR²—C(O)—, R is not a —CO-bonded substituent;
when X is -Alk-NR²—C(O)—, —NR²—C(O)— and R is an O—C₁₋₈-alkylated carboxyl-C₀₋₈-alkyl, C₁₋₈-alkyl, C₀₋₈-alkylcarbonyl-(N—C₀₋₈-alkyl)amino-C₁₋₈-alkyl, optionally N-mono- or N,N-di-C₁₋₈-alkylated or -arylated amino-C₁₋₈-alkyl, or C₃₋₈-cycloalkyl-C₀₋₈-alkylated or heterocyclyl-C₀₋₈-alkylated carbamoyl-CO₀₋₈-alkyl, each of which is optionally substituted, —C(O)—R¹ is not a substituent which stems from a carbonylation reagent;
when X is -Alk-NR²—, —NR²-Alk- or —NR²—CO-Alk- and R¹ is an optionally substituted aryl, R is not a diphenylmethyl substituent or a phenyl(heteroaryl)methyl substituent, each of which is optionally substituted;
when X is -Alk-NR²—S(O)₂—, R is not an optionally N-mono- or N,N-di-C₁₋₈-alkylated or -arylated amino-C₁₋₈-alkyl substituent;
when X is —NR²—C(O)— and R¹ is an optionally substituted aryl, R is not a C₂₋₈-alkenyl substituent, a C₁₋₈-alkyl substituent, a C₂₋₈-alkynyl substituent or a C₃₋₈-cycloalkyl-C₁₋₈-alkyl substituent, each of which is optionally substituted;
when R is C₁₋₈-alkyl- or arylcarbonyl-(N—C₀₋₈-alkyl)amino-C₁₋₈-alkyl, X is not -Alk-;
when X is -Alk-C(O)—NR²— and R¹ is a phenyl substituted by 0-2 methoxy, R is not a C₀₋₈-alkyl- or arylcarbonyl-(N—C₀₋₈-alkyl)amino-C₁₋₈-alkyl substituent or optionally O—C₁₋₈-alkylated carboxyl-C₀₋₈-alkyl substituent;
when X is -Alk- and R¹ is an amino-substituted monocyclic nitrogen heteroaryl which may optionally be substituted, R is not a C₁₋₈-alkyl;
when X is -Alk-, —C(O)—NR²— or —NR²—C(O)— and R¹ is an optionally substituted pyridine, R is not a C₁₋₈-alkyl substituent, an optionally O—C₁₋₈-alkylated carboxyl-C₀₋₈-alkyl substituent or an optionally N-mono- or N,N-di-C₃₋₈-cycloalkyl-C₀₋₈-alkylated carbamoyl-C₀₋₈-alkyl substituent;
when R is an optionally substituted O—C₁₋₈-alkylated carboxyl-C₀₋₈-alkyl, R¹ is not a 4-aminopyrido[3,2-d]pyrimidin-6-yl substituent, a 4-aminopyrido[3,4-d]pyrimidin-6-yl substituent, a 4-aminopyrido[4,3-d]pyrimidin-7-yl substituent, a 4-aminopyrido[2,3-d]pyrimidin-7-yl substituent, a 4-aminopyrimido[4,5-d]pyrimidin-7-yl substituent, or a 4-aminopyrimido[5,4-d]pyrimidin-6-yl substituent, each of which is optionally substituted;
when X is —C(O)—NR²— and R¹ is an optionally substituted 2-benzothiazole, R is not a C₁₋₈-alkyl substituent, a C₁₋₈-haloalkyl substituent or a carboxyl-C₀₋₈-alkyl substituent;
when X is —NR²-Alk- and R¹ is phenyl, R is not an optionally substituted C₀₋₈-alkylated carbamoyl-C₀₋₈-alkyl substituent or an optionally N-mono- or N,N-di-C₁₋₈-alkylated or -arylated amino-C₁₋₈-alkyl substituent;
when X is -Alk-O— or -Alk-NR²— and R¹ is pyrazolyl, R is not C₁₋₈-alkyl;
when X is —NR²-Alk- and R¹ is optionally substituted phenyl, R is not a hydroxy- or C₁₋₈-alkoxy-substituted C₁₋₈-alkyl substituent;
when X is —NR²—C(O)— or —NR²—C(O)-Alk- and R is indol-3-yl-C₁₋₈-alkyl, R¹ is not an aryl substituent;
when X is -Alk- and R¹ is an optionally substituted phenyl, R is not an optionally hydroxy-, cyano-, C₁₋₈-alkylcarbonyl- or aryl-substituted C₂₋₈-alkenyl, C₁₋₈-alkyl, C₂₋₈-alkynyl, C₃₋₈-cycloalkyl-C₁₋₈-alkyl or optionally O—C₁₋₈-alkylated carboxyl-C₀₋₈-alkyl;
when X is -Alk-O—, —O-Alk-, Alk-S—, —S-Alk-, Alk-NR²—, —NR²-Alk-, —CO—NR²— or —NR²—CO— and R is C₁₋₈-alkyl, R¹ is not a 9-membered [4.3.0]-bicycle;
when X is —CO—NR²— and R¹ is an optionally substituted aryl, R is not an optionally substituted C₁₋₈-alkyl substituent, a carbamoyl substituent, a heteroarylcarbonyl substituent or an optionally N-mono- or N,N-di-C₁₋₈-alkylated sulphamoyl substituent;
when X is —NR²—CO— and R is a C₁₋₈-alkyl, R¹ is not an optionally substituted 1-naphthyl;
when X is —S-Alk- or —O-Alk- and R¹ is an optionally substituted aryl, R is not a C₁₋₈-alkyl substituent, an optionally N-mono- or N,N-di-C₁₋₈-alkylated amino-C₁₋₈-alkyl substituent, or an optionally O—C₁₋₈-alkylated carboxyl-C₀₋₈-alkyl substituent, each of which is optionally substituted by hydroxyl, alkoxy or oxo;
when X is —NR²—(CH)₂, R is not a C₁₋₈-alkyl substituent, an optionally N-mono- or N,N-di-C₁₋₈-alkylated amino-C₁₋₈-alkyl substituent or an optionally O—C₁₋₈-alkylated carboxyl-C₀₋₈-alkyl substituent, each of which is optionally substituted by hydroxyl, alkoxy or oxo;
when X is -Alk-NR²—CO— and R¹ is an optionally substituted 3-quinolin-4-olyl, R is not C₁₋₈-alkyl;
when R is an optionally O—C₁₋₈-alkylated carboxyl-C₀₋₈-alkyl or an optionally N-mono- or N,N-di-C₁₋₈-alkylated, C₃₋₈-cycloalkyl-C₀₋₈-alkylated or heterocyclyl-C₀₋₈-alkylated carbamoyl-C₀₋₈-alkyl, X is not -Alk-;
when X is —O-Alk-C(O)—NR²—, -Alk-C(O)—NR²—, —C(O)—NR²—, -Alk-S(O)₂—NR²— or —S(O)₂—NR²—, R is not a C₁₋₈-alkyl substituent, an optionally N-mono- or N,N-di-C₁₋₈-alkylated or -arylated amino-C₁₋₈-alkyl substituent, or a C₃₋₈-cycloalkyl-C₁₋₈-alkyl substituent, each of which are optionally substituted; and
when X is —O-Alk- or -Alk-O— and R¹ is an optionally substituted phenyl, R is not a hydroxy-substituted C₀₋₈-alkylcarbonyl-(N—C₀₋₈-alkyl)amino-C₁₋₈-alkyl substituent.

20. A compound according to claim 19, wherein
R is C₁₋₈-alkyl, C₀₋₈-alkylcarbonyl-(N—C₀₋₈-alkyl)amino-C₁₋₈-alkyl, C₃₋₈-cycloalkyl-C₁₋₈-alkyl, optionally N-mono- or N,N-di-C₃₋₈-cycloalkyl-C₀₋₈-alkylated or optionally N-mono- or N,N-di-heterocyclyl-C₀₋₈-alkylated carbamoyl-C₀₋₈-alkyl or C₃₋₈-cycloalkyl-C₀₋₈-alkylcarbonyl-(N—C₀₋₈-alkyl)amino-C₁₋₈-alkyl or heterocyclylcarbonyl-C₀₋₈-alkyl, each of which is either unsubstituted or substituted by 1-4 C₁₋₈-alkoxy, C₁₋₈-alkoxy-C₁₋₈-alkoxy, C₁₋₈-alkoxycarbonyl-(N—C₁₋₈-alkyl)amino, C₁₋₈-alkyl, C₁₋₈-alkylcarbonyl, C₀₋₈-alkylcarbonyl-(N—C₀₋₈-alkyl)amino, C₁₋₈-alkylsulphanyl, C₁₋₈-alkylsulphinyl, C₁₋₈-alkylsulphonyl, aryl-C₀₋₈-alkoxy, aryl, arylamino, aryl-C₀₋₈-alkylsulphonyl, cyano, C₃₋₈-cycloalkoxy, halogen, heterocyclyl, heterocyclyl-C₀₋₈-alkoxy, heterocyclyl-C₀₋₈-alkylamino, heterocyclylcarbonyl, hydroxyl, phosphonyl, optionally N-mono- or N,N-di-C₁₋₈-alkylated amino, optionally N-mono- or N,N-di-C₁₋₈-alkylated carbamoyloxy, optionally N-mono- or N,N-di-C₁₋₈-alkylated sulphamoyl, optionally N-mono-, -di- or -tri-C₁₋₈-alkylated or heterocyclyl-substituted ureido, optionally N-mono- or N,N-di-C₁₋₈-alkylated, N-mono- or N,N-di-arylated or N-mono- or N,N-di-heterocyclyl-C₀₋₈-alkylated amino-C₀₋₈-alkylcarbonyl, oxo or trifluoromethyl.

21. A compound according to claim 19, wherein R¹ is benzimidazolyl, benzo[1,3]dioxolyl, benzofuranyl, benzoxazolyl, benzothiazolyl, benzo[b]thienyl, quinazolinyl, quinolyl, quinoxalinyl, 2H-chromenyl, carbazolyl, dihydro-2H-benzo[1,4]oxazinyl, dihydro-3H-benzo[1,4]oxazinyl, dihydro-2H-benzo[1,4]thiazinyl, 2,3-dihydroindolyl, dihydro-1H-pyrido[2,3-b][1,4]oxazinyl, imidazo[1,2-a]pyridyl, imidazo[1,5-a]pyridyl, indazolyl, indolyl, isobenzofuranyl, isoquinolyl, [1,5]naphthyridyl, phenyl, phthalazinyl, pyridyl, pyrimidinyl, 1H-pyrrolo[2,3-b]pyridyl, 1H-pyrrolo[2,3-c]pyridyl, 1H-pyrrolo[3,2-b]pyridyl, tetrahydroquinolyl, tetrahydroquinoxalinyl, tetrahydroimidazo[1,2-a]pyridyl, tetrahydroimidazo[1,5-a]pyridyl, tetrahydroisoquinolyl, [1,2,3]triazolo[1,5-a]pyridyl or [1,2,4]triazolo[4,3-a]pyridyl, each of which is substituted by 1-4 C₁₋₈-alkanoyl, C₁₋₈-alkoxy, C₁₋₈-alkoxy-C₁₋₈-alkanoyl, C₁₋₈-alkoxy-C₁₋₈-alkoxy, C₁₋₈-alkoxy-C₁₋₈-alkoxy-C₁₋₈-alkyl, C₁₋₈-alkoxy-C₁₋₈-alkyl, C₁₋₈-alkoxycarbonylamino-C₁₋₈-alkoxy, C₁₋₈-alkoxycarbonylamino-C₁₋₈-alkyl, C₁₋₈-alkyl, C₀₋₈-alkylcarbonylamino-C₁₋₈-alkoxy, C₀₋₈-alkylcarbonylamino-C₈-alkyl, aryl-C₀₋₈-alkoxy, aryl-C₀₋₈-alkyl, cyano, cyano-C₁₋₈-alkoxy, cyano-C₁₋₈-alkyl, halogen, halogen-C₁₋₈-alkoxy, halogen-C₁₋₈-alkyl, halogen-aryl, heterocyclyl-C₀₋₈-alkoxy, heterocyclyl-C₀₋₈-alkyl, oxide or oxo,
whereby, when R¹ is heterocyclyl and contains at least one saturated carbon atom, this heterocyclyl radical may additionally be substituted on a saturated carbon atom by a C₂₋₈-alkylene chain whose two ends are fixed on this saturated carbon atom and thus form a spirocycle, whereby one CH₂ group of the alkylene chain may be replaced by oxygen.

22. A compound according to claim 19, wherein
R is C₂₋₈-alkenyl, C₂₋₈-alkynyl, C₁₋₈-alkyl, C₀₋₈-alkylcarbonyl-(N—C₀₋₈-alkyl)amino-C₁₋₈-alkyl, optionally N-mono- or N,N-di-C₁₋₈-alkylated or -arylated amino-C₁₋₈-alkyl, optionally O—C₁₋₈-alkylated carboxyl-C₀₋₈-alkyl, C₃₋₈-cycloalkyl-C₁₋₈-alkyl, optionally N-mono- or N,N-di-C₃₋₈-cycloalkyl-C₀₋₈-alkylated or optionally N-mono- or N,N-di-heterocyclyl-C₀₋₈-alkylated carbamoyl-C₀₋₈-alkyl, C₃₋₈-cycloalkyl-C₀₋₈-alkylcarbonyl-(N—C₀₋₈-alkyl)amino-C₁₋₈-alkyl, arylcarbonyl-(N—C₀₋₈-alkyl)amino-C₁₋₈-alkyl, optionally N-mono- or N,N-di-C₁₋₈-alkylated or -arylated sulphamoyl-C₀₋₈-alkyl, C₁₋₈-alkylsulphonyl-C₀₋₈-alkyl or heterocyclylcarbonyl-C₀₋₈-alkyl, each of which is either unsubstituted or substituted by 1-4 C₁₋₈-alkoxy, C₁₋₈-alkoxy-C₁₋₈-alkoxy, C₁₋₈-alkoxycarbonyl-(N—C₁₋₈-alkyl)amino, C₁₋₈-alkyl, C₁₋₈-alkylcarbonyl, C₀₋₈-alkylcarbonyl-(N—C₀₋₈-alkyl)amino, C₁₋₈-alkylsulphanyl, C₁₋₈-alkylsulphinyl, C₁₋₈-alkylsulphonyl, aryl-C₀₋₈-alkoxy, aryl, arylamino, aryl-C₀₋₈-alkylsulphonyl, cyano, C₃₋₈-cycloalkoxy, halogen, heterocyclyl, heterocyclyl-C₀₋₈-alkoxy, heterocyclyl-C₀₋₈-alkylamino, heterocyclylcarbonyl, hydroxyl, phosphonyl, optionally N-mono- or N,N-di-C₁₋₈-alkylated amino, optionally N-mono- or N,N-di-C₁₋₈-alkylated carbamoyloxy, optionally N-mono- or N,N-di-C₁₋₈-alkylated sulphamoyl, optionally N-mono-, -di- or -tri-C₁₋₈-alkylated or heterocyclyl-substituted ureido, optionally N-mono- or N,N-di-C₁₋₈-alkylated, N-mono- or N,N-di-arylated or N-mono- or N,N-di-heterocyclyl-C₀₋₈-alkylated amino-C₀₋₈-alkylcarbonyl, oxo or trifluoromethyl;
R¹ is 2H-chromenyl, 3,4-dihydro-2H-benzo[1,4]oxazinyl or carbazolyl, each of which is substituted by 1-4 C₁₋₈-alkoxy, C₁₋₈-alkoxy-C₁₋₈-alkoxy, C₁₋₈-alkoxy-C₁₋₈-alkoxy-C₁₋₈-alkyl, C₁₋₈-alkoxy-C₁₋₈-alkyl, C₁₋₈-alkoxycarbonylamino-C₁₋₈-alkoxy, C₁₋₈-alkoxycarbonylamino-C₁₋₈-alkyl, C₁₋₈-alkyl, C₀₋₈-alkylcarbonylamino-C₁₋₈-alkoxy, C₀₋₈-alkylcarbonylamino-C₁₋₈-alkyl, halogen, halogen-C₁₋₈-alkoxy, halogen-C₁₋₈-alkyl, halogen-aryl or oxo,
whereby a saturated carbon atom of the heterocyclyl radical may additionally be substituted on the saturated carbon atom by a C₂₋₈-alkylene chain whose two ends are fixed on this saturated carbon atom and thus form a spirocycle, whereby one CH₂ group of the alkylene chain may be replaced by oxygen, and
X is —O—CH₂—.

23. A compound according to claim 19, wherein
R is C₁₋₈-alkyl, C₀₋₈-alkylcarbonylamino-C₁₋₈-alkyl, C₃₋₈-cycloalkyl-C₁₋₈-alkyl, optionally N-mono-C₃₋₈-cycloalkyl-C₀₋₈-alkylated, optionally N-mono-heterocyclyl-C₀₋₈-alkylated carbamoyl-C₀₋₈-alkyl, C₃₋₈-cycloalkyl-C₀₋₈-alkylcarbonylamino-C₁₋₈-alkyl or heterocyclylcarbonyl-C₀₋₈-alkyl, each of which is either unsubstituted or substituted by 1-2 heterocyclyl or optionally N-mono- or N,N-di-C₁₋₈-alkylated, N-mono- or N,N-di-arylated or N-mono- or N,N-di-heterocyclyl-C₀₋₈-alkylated carbamoyl, wherein the heterocyclyl is particularly preferably a saturated heterocyclyl preferably selected from the group comprising tetrahydropyranyl, morpholinyl, piperidinyl, tetrahydrofuranyl, 3-oxa-bicyclo[3.1.0]hexanyl and 6-oxaspiro[2.5]octanyl which heterocyclyl substituents may be further substituted;
R¹ is 2H-chromenyl, 3,4-dihydro-2H-benzo[1,4]oxazinyl or carbazolyl, each of which is substituted by 1-4 C₁₋₈-alkoxy, C₁₋₈-alkoxy-C₁₋₈-alkoxy, C₁₋₈-alkoxy-C₁₋₈-alkoxy-C₁₋₈-alkyl, C₁₋₈-alkoxy-C₁₋₈-alkyl, C₁₋₈-alkoxycarbonylamino-C₁₋₈-alkoxy, C₁₋₈-alkoxycarbonylamino-C₁₋₈-alkyl, C₁₋₈-alkyl, C₀₋₈-alkylcarbonylamino-C₁₋₈-alkoxy, C₀₋₈-alkylcarbonylamino-C₁₋₈-alkyl, halogen, halogen-C₁₋₈-alkoxy, halogen-C₁₋₈-alkyl, halogen-aryl or oxo,
whereby a saturated carbon atom of the heterocyclyl radical may additionally be substituted on the saturated carbon atom by a C₂₋₈-alkylene chain whose two ends are fixed on this saturated carbon atom and thus form a spirocycle, whereby one CH₂ group of the alkylene chain may be replaced by oxygen; and
X is —O—CH₂—.

24. A compound according to any one of claims 19-23, wherein the compound of general formula (I) is a compound of the general formula (IA) 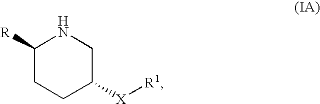
or a pharmaceutically usable salt thereof, wherein R, R¹ and X are each as defined for the compounds of the formula (I) according to any one of claims 19 to 23, respectively.

25. A method for producing a medicament, which comprises formulating the compound of the general formula (I) according to claim 19, or its pharmaceutically usable salt or compound, whereby one or more atoms have been replaced by their stable, non-radioactive isotopes, and wherein R, R¹ and X are each as defined for the compound of the formula (I) according to claim 19, as an active ingredient.

26. A method for preventing, for retarding the progression of or for treating hypertension, heart failure, glaucoma, myocardial infarction, kidney failure, restenoses or stroke, which comprises administering, to a subject in need thereof, a therapeutically effective amount of a compound of the general formula (I) according to claim 19, or its pharmaceutically usable salt or compound, in which one or more atoms have been replaced by their stable, non-radioactive isotopes, and wherein R, R¹ and X are each as defined for the compound of the formula (I) according to claim 19.

27. A method according to claim 25 or 26, wherein the compound of general formula (I) is a compound of the general formula (IA) 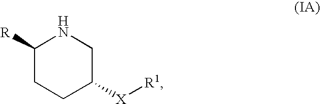
or its pharmaceutically usable salt or compound, in which one or more atoms have been replaced by their stable, non-radioactive isotopes, and wherein R, R¹ and X are each as defined for the compound of the formula (I) according to claim 25 or 26, respectively.

28. A method according to claim 14, wherein the compound of general formula (I) is a compound of the general formula (IA) 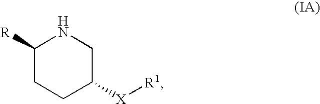
or a pharmaceutically usable salt thereof, in which R, R¹ and X are each as defined for the compound of the formula (I) according to claim 14.

29. A composition according to claim 17, wherein the compound of general formula (I) is a compound of the general formula (IA) 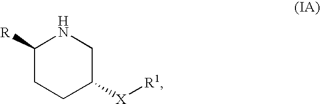
or a pharmaceutically usable salt thereof, in which R, R¹ and X are each as defined for the compound of the formula (I) according to claim 17.
